SEQUENCE LISTING (13 sequences)

SEQ ID NO: 1 - DNA, 25 bp, Homo sapiens
gggaagaaatcctccagaagaaaga

SEQ ID NO: 2 - DNA, 24 bp, Homo sapiens
gagatccgagagaaaacagccttt

SEQ ID NO: 3 - DNA, 19 bp, Homo sapiens
cagtgctcacctctgattg

SEQ ID NO: 4 - DNA, 24 bp, Homo sapiens
atgtttggcctcaaaagaaacgcg

SEQ ID NO: 5 - DNA, 23 bp, Homo sapiens
ggctatcttattagatatgccaa

SEQ ID NO: 6 - DNA, 20 bp, Homo sapiens
cttctacaatgagctgcgtg

SEQ ID NO: 7 - DNA, 20 bp, Homo sapiens
tcatgaggtagtcagtcagg

SEQ ID NO: 8 - DNA, 17 bp, Homo sapiens
gcctggctgaggtgtgc

SEQ ID NO: 9 - DNA, 16 bp, Homo sapiens
gcgggagaggcgatga

SEQ ID NO: 10 - DNA, 19 bp, Homo sapiens
tgcagagaagatgtggcca

SEQ ID NO: 11 - DNA, 20 bp, Homo sapiens
atgcttcggaaactggacat

SEQ ID NO: 12 - DNA, 19 bp, Homo sapiens
ccttccaaggatgggtttg

SEQ ID NO: 13 - DNA, 19 bp, Homo sapiens
ctaggttgctagggtgcaa

(19) United States
(12) Patent Application Publication
(10) Pub. No.: US 2009/0246773 A1
(43) Pub. Date: Oct. 1, 2009

(54) DIAGNOSTIC COMPOSITIONS AND TREATMENT METHODS FOR CONDITIONS INVOLVING TROPHOBLAST CELL DEATH, DIFFERENTIATION, INVASION AND/OR CELL FUSION AND TURNOVER

(75) Inventors: Isabella Caniggia, Toronto (CA); Alessandro Rolfo, Toronto (CA); Martin Post, Toronto (CA)

Correspondence Address: HOWSON & HOWSON LLP, 501 OFFICE CENTER DRIVE, SUITE 210, FORT WASHINGTON, PA, 19034, US

(73) Assignee: Mount Sinai Hospital, Toronto (CA)

(21) Appl. No.: 12/282,395

(22) PCT Filed: Mar. 9, 2007
(86) PCT No.: PCT/CA2007/000394

§ 371 Date: Feb. 19, 2009

Related U.S. Application Data
(60) Provisional application No. 60/781,457, filed on Mar. 10, 2006.
(60) Provisional application No. 60/899,836, filed on Feb. 6, 2007.

(51) Int. Cl. C12Q 1/68; G01N 33/53

(52) U.S. Cl. 435/6; 435/7.1

(57) ABSTRACT

The invention provides a method for diagnosing in a subject a condition requiring modulation of or involving trophoblast cell death, differentiation, invasion, and/or cell fusion and turnover, comprising detecting HIF Iα and the factors which modulate or are modulated by this protein, specifically TGFβ3, sFLT, VEGF, SMAD2, 3 and 7, MtdP, MtdL, MclI 1, MclIc, VHL, SiahI, Siah2, ENG, and PHD. The invention also provides a method for diagnosing or distinguishing in a subject a specific condition requiring modulation of or involving trophoblast cell death, differentiation, invasion, and/or cell fusion and turnover, in particular early onset severe preeclampsia (EPE), late onset preeclampsia (LPE) and mtre-uterme growth restriction (IUGR) comprising detecting HLF Iα and the factors which modulate or are modulated by this protein as defined above.

### FIELD OF THE INVENTION

The invention relates to diagnostic compositions and treatment methods for conditions associated with trophoblast cell death, differentiation, invasion, and/or cell fusion and turnover.

### BACKGROUND OF THE INVENTION

Considerable evidence supports a central role for local oxygen availability in human trophoblast cell differentiation and hence appropriate placental development and function (Kingdom, 1999). During early pregnancy, entry of maternal blood to the inter-villous space of the placenta is limited by the endovascular trophoblast and thus oxygen levels around the interstitial and villous trophoblast is relatively low (˜15 mmHg, equivalent to 2-3% O₂) (Jaunieux, 2000). This low oxygen environment is essential for normal embryonic development (Hempstock, 2003). After 10-12 weeks of gestation, maternal blood flow to the intervillous space increases and oxygen levels surrounding the placental villi reaches ˜55 mmHg (approximately 8% O₂) (Jaunieux, 2000). Uteroplacental blood flow rises exponentially in the second trimester of pregnancy and is associated with transformation of the proximal uterine artery Doppler waveform (Thaler, 1990). Failure of this process to occur results in uteroplacental vascular insufficiency and chronic hypoxia, placing the pregnancy at risk of preterm delivery from preeclampsia (Lunell, 1982) and/or intrauterine growth restriction (IUGR) (Vierro, 2004). The main cellular pathway by which oxygen regulates gene expression is the formation of the heteromeric protein complex known as hypoxia inducible factor (HIF). HIF is comprised of two distinct subunits: α and β. When oxygen tension is low, the labile α subunit forms a heterodimer with the constitutively expressed β subunit. The heterodimer is subsequently translocated inside the nucleus where it binds to short DNA motifs (known as HRE's: Hypoxia Responsive Elements) in the promoter regions of a variety of genes thereby activating their transcription. Under normoxia, HIF-1α is rapidly hydroxylated and hence targeted for proteosomal degradation (Metzen, 2004).

It would be useful to distinguish between different conditions involving trophoblast cell death, differentiation, invasion, and/or cell fusion and turnover in order to design appropriate clinical approaches to prevent or treat these conditions.

### SUMMARY OF THE INVENTION

Broadly stated the invention provides a method for diagnosing in a subject a condition requiring modulation of or involving trophoblast cell death, differentiation, invasion, and/or cell fusion and turnover, comprising detecting factors that modulate or are modulated by HIF1α. The invention also provides a method for diagnosing or distinguishing in a subject a specific condition requiring modulation of or involving trophoblast cell death, differentiation, invasion, and/or cell fusion and turnover comprising detecting factors that modulate or are modulated by HIF1α.

“Conditions” referred to herein include conditions, diseases or disorders requiring modulation of, or involving, trophoblast cell death, differentiation, invasion, and/or cell fusion and turnover. Examples of such conditions are preeclampsia including early onset severe preeclampsia (EPE) and late onset preeclampsia (LPE), intra-uterine growth restriction (IUGR), choriocarcinoma, hydatiform mole, and molar pregnancy.

In aspects of the invention, factors are selected from two or three of the following classes:
i) polypeptides that increase tissue oxygen delivery by their systemic participation in erythropoiesis (transferrin) and/or polynucleotides encoding the polypeptides.
ii) polypeptides that increase local oxygen delivery to tissue via modification of blood vessel relaxation and development and/or polynucleotides encoding the polypeptides.
iii) polypeptides that do not alter tissue oxygen delivery but are necessary for adaptation of cellular metabolism under conditions of low oxygen and/or polynucleotides encoding the polypeptides.

Polypeptide factor(s) are referred to herein as “Polypeptide Marker(s)” and polynucleotide factor(s) (i.e. polynucleotides encoding polypeptide factors) are referred to herein as “Polynucleotide Marker(s)”. Polypeptide Markers and Polynucleotide Markers are collectively referred to herein as “Marker(s)”.

In aspects of the invention, the Markers are SMAD2, phospho-SMAD2, SMAD-3, phospho-SMAD3, SMAD7, and/or ceruloplasmin, and optionally one or more of a Mtd polypeptide (e.g. Mtd-L, Mtd-S and/or Mtd-P) (Soleymanlou N. et al, Cell Death Differ 12:441-452, 2005); myeloid cell leukemia factor-1 (Mcl-1) isoforms or caspase cleaved Mcl-1 isoforms, transforming growth factor β3 (TGFβ3) (Caniggia, 1, et al, J Clin Invest. 1999 June; 103 (12): 1641-50), endoglin (ENG), hypoxia inducible transcription factors-1alpha and -2alpha (HIF-1α, HIF-2α) (Caniggia, I. et al. Placenta. 2000 March-April; 21 Suppl A: S25-30 Wang, G L, et al, Proc Natl Acad Sci USA 1995: 92:5510-4; Biol Reprod 2001; 64:499-506; Biol Reprod 2001; 64:1019-1020), prolyl hydroxylating domain-containing 1 (PHD1), prolyl hydroxylating domain-containing 2 (PHD2), prolyl hydroxylating domain-containing 3 (PHD3) (Epstein, A C, et al, Cell 2001; 107:43-54), E3 ligases Siah1/2 (Nakayama, K. and Z. Ronai, Cell Cycle 3:11, 1345-1347), sFlt (US Published Patent No. 20040126828), VHL, cullin 2, neural precursor cell expressed, developmentally down-regulated 8 (NEDD8), syncytin, Fas, VEGF, FIH, cleaved caspase (e.g., caspase-3), and/or p53, and polynucleotides encoding any of these polypeptides.

In particular aspects of the invention, the Markers comprise or are selected from the group consisting of a SMAD2, phospho-SMAD2, SMAD-3, phospho-SMAD3, SMAD7, ceruloplasmin, sFlt, Mtd-L, Mtd-S, Mtd-P, Mcl-1 isoforms (in particular Mcl-1S or Mcl-1L, or caspase cleaved Mcl-1S or Mcl-1L, in particular caspase cleaved Mcl-1L), TGFβ3, endoglin, HIF1α, HIF-2α, PHD1, PHD2, PHD3, VHL, Siah1/2, cullin 2, NEDD8, VEGF, FIH, syncytin, cleaved caspase (e.g., caspase-3), Fas, and/or p53, and/or polynucleotides encoding any of these polypeptides.

In embodiments of the invention, the Markers comprise or are selected from the group consisting of a SMAD2, phospho-SMAD2, SMAD-3, phospho-SMAD3, SMAD7, sFlt, Mtd-L, Mtd-P, Mcl-1c, Mcl-1L, TGFβ3, endoglin, HIF1α, PHD1, PHD2, PHD3, VHL, Siah1/2, and VEGF and/or polynucleotides encoding any of these polypeptides.

In embodiments of the invention, the Markers are the biomarkers identified in Table 2.

In an aspect of the invention a method is provided for diagnosing in a subject a condition requiring modulation of or involving trophoblast cell death, differentiation, invasion, and/or cell fusion and turnover, comprising detecting one or more of a SMAD2, phospho-SMAD2, SMAD-3, phospho-SMAD3, SMAD7, and ceruloplasmin, and/or polynucleotides encoding one or more of a SMAD2, phospho-SMAD2, SMAD-3, phospho-SMAD3, SMAD7, and ceruloplasmin, in a sample from the subject. In an embodiment of the diagnostic method of the invention, a method is provided for diagnosing increased risk of preeclampsia in a subject comprising detecting one or more of a SMAD2, phospho-SMAD2, SMAD-3, phospho-SMAD3, SMAD7, and ceruloplasmin, or a polynucleotide encoding same, in a sample from the subject.

The invention provides a set of Markers that can distinguish conditions requiring modulation of or involving trophoblast cell death, differentiation, invasion, and/or cell fusion and turnover, in particular early onset severe preeclampsia, late onset preeclampsia, and IUGR. Methods are provided for use of these Markers to distinguish between the patient groups, and to determine general courses of treatment.

In an aspect, the invention relates to a method of characterizing a biological sample by detecting or quantitating in the sample one or more Polynucleotide Markers extracted from the sample that are characteristic of a condition requiring modulation of or involving trophoblast cell death, differentiation, invasion, and/or cell fusion and turnover (e.g. preeclampsia), the method comprising assaying for differential expression of Polynucleotide Markers in the sample. Differential expression of the Polynucleotide Markers can be determined by micro-array, hybridization or by amplification of the extracted polynucleotides.

The invention also relates to a method of characterizing or classifying a sample by detecting or quantitating in the sample one or more Polypeptide Markers extracted from the sample that are characteristic of a condition requiring modulation of or involving trophoblast cell death, differentiation, invasion, and/or cell fusion and turnover (e.g. preeclampsia), the method comprising assaying for differential expression of Polypeptide Markers in the sample. Differential expression of Polypeptide Markers can be assayed using procedures known in the art, including without limitation, separation techniques known in the art, antibody microarrays, or mass spectroscopy of polypeptides extracted from a sample.

An aspect of the invention is directed to bioinformatic methods for analyzing gene expression data generated from nucleic acid micro-array experiments to identify further markers from various cell types. Another embodiment of the invention is directed to marker genes identified from mammalian (e.g., human, primate) peripheral blood cells at normal and/or abnormal states. The biomarker genes are useful as molecular targets for therapeutics of a disorder or disease in mammals.

The invention contemplates a gene expression “signature” that is associated with a condition requiring modulation of or involving trophoblast cell death, differentiation, invasion, and/or cell fusion and turnover (e.g. preeclampsia). This signature provides a highly sensitive and specific test with both high positive and negative predictive values permitting diagnosis and prediction of the condition.

The invention provides gene marker sets that distinguish different conditions requiring modulation of or involving trophoblast cell death, differentiation, invasion, and/or cell fusion and turnover (e.g. early and late onset preeclampsia and IUGR) and uses therefor. A genetic marker set may comprise a plurality of genes comprising or consisting of at least 2, 5, 10, 15, 20, 25, 30, 35, 40, 45, or 50 of the genes corresponding to the biomarkers listed in Table 2. In an aspect, the gene marker sets comprise gene clusters which may be represented by dendograms, or comprise genes in pathways of up and/or down regulated genes identified in accordance with the invention.

The present invention further relates to a method for detecting, preventing, and/or treating a condition requiring regulation of trophoblast cell death, differentiation, invasion, and/or cell fusion and turnover by modulating a SMAD2, phospho-SMAD2, SMAD-3, phospho-SMAD3, SMAD7, and/or ceruloplasmin, or a polynucleotide encoding same. In an embodiment, a method for treating cancer is provided comprising administering to a patient in need thereof, a SMAD2, phospho-SMAD2, SMAD-3, phospho-SMAD3, SMAD7, and/or ceruloplasmin and/or polynucleotide encoding same or an agonist or antagonist thereof. In another embodiment, a method for treating preeclampsia is provided comprising administering to a patient in need thereof an inhibitor of a SMAD2, phospho-SMAD2, SMAD-3, phospho-SMAD3 and/or SMAD7, and/or polynucleotides encoding same (e.g. antisense, micRNA molecule, or a composition of the invention).

The invention also contemplates a method for regulating trophoblast cell death, differentiation, invasion, and/or cell fusion and turnover comprising inhibiting or stimulating a SMAD2, phospho-SMAD2, SMAD-3, phospho-SMAD3, SMAD7, and/or ceruloplasmin, and/or polynucleotides encoding a SMAD2, phospho-SMAD2, SMAD-3, phospho-SMAD3, SMAD7 and/or ceruloplasmin.

In an embodiment of the invention, a method is provided for reducing trophoblast cell death, differentiation, invasion, and/or cell fusion and turnover in a subject comprising administering to the subject an effective amount of a modulator (e.g. inhibitor) of a SMAD2, phospho-SMAD2, SMAD-3, phospho-SMAD3, SMAD7, and/or ceruloplasmin, and/or a polynucleotide encoding same. In a preferred embodiment of the invention a method is provided for treating a woman suffering from, or who may be susceptible to preeclampsia comprising administering therapeutically effective dosages of an inhibitor of a SMAD2, phospho-SMAD2, SMAD-3, phospho-SMAD3, SMAD7 and/or ceruloplasmin, and/or a polynucleotide encoding same. A therapeutically effective dosage is an amount of an inhibitor effective to down regulate or inhibit a SMAD2, phospho-SMAD2, SMAD-3, phospho-SMAD3, SMAD7, and/or ceruloplasmin, and/or a polynucleotide encoding same, in the woman.

In another embodiment of the invention, a method is providing for reducing trophoblast cell death, differentiation, invasion, and/or cell fusion and turnover in a subject comprising administering an effective amount of a SMAD2, phospho-SMAD2, SMAD-3, phospho-SMAD3, SMAD7, and/or ceruloplasmin, and/or polynucleotides encoding same, or a stimulator of same. In a preferred embodiment, a method is provided for monitoring or treating choriocarcinoma or hydatiform mole in a subject comprising administering therapeutically effective dosages of a SMAD2, phospho-SMAD2, SMAD-3, phospho-SMAD3, SMAD7, and/or ceruloplasmin, and/or polynucleotides encoding same, or a stimulator of same. An amount is administered which is effective to up-regulate or stimulate a SMAD2, phospho-SMAD2, SMAD-3, phospho-SMAD3, SMAD7, and/or ceruloplasmin and/or polynucleotides encoding same in the subject.

A method of the invention may in the alternative or additionally comprise inhibiting or stimulating other polypeptides associated with regulating trophoblast cell death, differentiation, invasion, and/or cell fusion and turnover [e.g., sFlt, a Mtd polypeptide (e.g. Mtd-L, Mtd-S and/or Mtd-P); Mcl-1 isoforms (in particular Mcl-1S, Mcl-1c, or Mcl-1L, or caspase cleaved Mcl-1S or Mcl-1L, in particular caspase cleaved Mcl-1L), TGFβ3, endoglin, HIF1α, PHD1, PHD2, PHD3, VHL, Siah1/2, cullin2, NEDD8, VEGF, FIH, syncytin, cleaved caspase (e.g., caspase-3), Fas, and/or p53].

The invention provides a diagnostic composition comprising an agent that binds to a polypeptide marker associated with a condition requiring regulation of trophoblast cell death, differentiation, invasion, and/or cell fusion and turnover, or hybridizes to a polynucleotide encoding such a polypeptide marker, wherein the marker comprises a SMAD2, phospho-SMAD2, SMAD-3, phospho-SMAD3, SMAD7, and/or ceruloplasmin, and/or polynucleotides encoding a SMAD2, SMAD-3, phospho-SMAD3, SMAD7, and ceruloplasmin.

The invention also provides a method for assessing the potential efficacy of a test agent for preventing, inhibiting, or reducing in a subject a condition requiring regulation of trophoblast cell death, differentiation, invasion, and/or cell fusion and turnover, the method comprising comparing: (a) levels of one or more Markers associated with the condition, wherein the markers comprise a SMAD2, phospho-SMAD2, SMAD-3, phospho-SMAD3, SMAD7, and/or ceruloplasmin and/or polynucleotides encoding a SMAD2, SMAD-3, phospho-SMAD3, SMAD7, and ceruloplasmin, in a first sample obtained from a subject and exposed to the test agent, and (b) levels of the markers in a second sample obtained from the subject, wherein the sample is not exposed to the test agent, wherein a significant difference in the levels of expression of the markers in the first sample, relative to the second sample, is an indication that the test agent is potentially efficacious for preventing, inhibiting or reducing the condition in the subject.

Further the invention provides a method of assessing the efficacy of a therapy for preventing, inhibiting, or reducing a condition requiring regulation of trophoblast cell death, differentiation, invasion, and/or cell fusion and turnover in a subject, the method comprising comparing: (a) in a first sample obtained from the subject levels of one or more markers associated with the condition wherein the markers comprise a SMAD2, phospho-SMAD2, SMAD-3, phospho-SMAD3, SMAD7, and/or ceruloplasmin and/or polynucleotides encoding SMAD2, SMAD-3, phospho-SMAD3, SMAD7, and/or ceruloplasmin; and (b) levels of the markers in a second sample obtained from the subject following therapy, wherein a significant difference in the levels of expression of the markers in the second sample, relative to the first sample, is an indication that the therapy is efficacious for preventing, inhibiting, or reducing symptoms of the condition in the subject.

In an aspect the invention provides a method of selecting an agent for preventing, inhibiting or reducing in a subject a condition requiring regulation of trophoblast cell death, differentiation, invasion, and/or cell fusion and turnover the method comprising (a) obtaining a sample containing one or more polypeptides and/or polynucleotides from the subject; (b) separately exposing aliquots of the sample in the presence of a plurality of test agents; (c) in each of the aliquots comparing levels of one or more markers associated with the condition wherein the markers comprise SMAD2, phospho-SMAD2, SMAD-3, phospho-SMAD3, SMAD7, and ceruloplasmin and/or polynucleotides encoding SMAD2, SMAD-3, phospho-SMAD3, SMAD7, and ceruloplasminin; and (d) selecting one of the test agents which alters the levels of markers in the aliquot containing that test agent, relative to other test agents.

In another aspect, the invention provides a method of preventing, inhibiting, or reducing a condition requiring regulation of trophoblast cell death, differentiation, invasion, and/or cell fusion and turnover in a subject, the method comprising (a) obtaining from the subject a sample containing one or more polypeptides and/or polynucleotides wherein the markers comprise a SMAD2, phospho-SMAD2, SMAD-3, phospho-SMAD3, SMAD7, and/or ceruloplasmin, and/or polynucleotides encoding SMAD2, SMAD-3, phospho-SMAD3, SMAD7, and/or ceruloplasminin; (b) separately maintaining aliquots of the sample in the presence of a plurality of test agents; (c) comparing levels of one or more markers in each of the aliquots; and (d) administering to the subject at least one of the test agents which alters the levels of markers in the aliquot containing that test agent, relative to other test agents.

In another aspect, the invention provides a method of assessing the potential of a test compound to cause a condition requiring regulation of trophoblast cell death, differentiation, invasion, and/or cell fusion and turnover, the method comprising: (a) maintaining separate aliquots of samples containing one or more polypeptides and/or polynucleotides in the presence and absence of the test compound; and (b) in each of the aliquots comparing expression of one or more markers associated with the condition wherein the markers comprise a SMAD2, phospho-SMAD2, SMAD-3, phospho-SMAD3, SMAD7, and/or ceruloplasmin, and/or polynucleotides encoding a SMAD2, SMAD-3, phospho-SMAD3, SMAD7, and/or ceruloplasminin; and, wherein a significant difference in levels of markers in the aliquot maintained in the presence of the test compound, relative to the aliquot maintained in the absence of the test compound, is an indication that the test compound potentially causes a condition requiring regulation of trophoblast cell death, differentiation, invasion, and/or cell fusion and turnover.

In embodiments of the invention, one, two, three or more of the following are detected: SMAD2, phospho-SMAD2, SMAD-3, phospho-SMAD3, SMAD7, ceruloplasmin, sFlt, Mtd-L, Mtd-S, Mtd-P, Mcl-1 isoforms (in particular Mcl-1S or Mcl-1L, or caspase cleaved Mcl-1S or Mcl-1L, in particular caspase cleaved Mcl-1L), TGFβ3, endoglin, HIF1α, HIF-2α, PHD1, PHD2, PHD3, VHL, Siah1/2, cullin 2, NEDD8, VEGF, FIH, syncytin, cleaved caspase (e.g., caspase-3), Fas, and/or p53, and/or polynucleotides encoding any of these polypeptides. In particular embodiments, one, two, three or more of the following are detected: SMAD2, phospho-SMAD2, SMAD-3, phospho-SMAD3, SMAD7, sFlt, Mtd-L, Mtd-P, Mcl-1c, Mcl-1L, TGFβ3, endoglin, HIF1α, PHD1, PHD2, PHD3, VHL, Siah1/2, and VEGF and/or polynucleotides encoding any of these polypeptides. In more particular embodiments one, two, three or more of biomarkers identified in Table 2 are detected.

The invention also relates to kits for carrying out the methods of the invention.

These and other aspects, features, and advantages of the present invention should be apparent to those skilled in the art from the following drawings and detailed description.

### DESCRIPTION OF THE DRAWINGS

The invention will now be described in relation to the drawings in which:

FIG. 1. sFlt-1 expression during placental development. (A) Expression of sFlt-1 transcript in early first trimester placental samples vs. later gestation as assessed by real-time PCR analysis (n=11 for each gestational age tested); *P<0.05, 5-9 weeks vs. 10-12 weeks. (B) Representative sFlt-1 immunoblot of first and early second trimester samples. 5-9 weeks, n=5, 10-12 w, n=5, 13-18 w n=7. (C) Immunolocalization of sFlt-1 in first trimester tissue. Positive staining is represented by brownish staining. (CT: Cytotrophoblasts; ST: Syncytiotrophoblast, SK: Syncytial knot; S: Stroma). All values are represented as the mean (±SE) of three independent experiments.

FIG. 2. sFlt-1 expression in high altitude and preeclamptic placental samples. (A) Expression of sFlt-1 mRNA in high altitude (HA) vs. sea level (SL) samples assessed by real-time PCR analysis. HA, n=15, SL, n=12. *P<0.05, HA vs. SL. (B) Fold change in the transcript level of sFlt-1 in early severe preeclampsia (PE; n=16) compared to age matched controls (AMC; n=12) assessed by real-time PCR. *P<0.05, PE vs. TC and vs. PTC. (C) Upper panel, representative sFlt-1 Western blot in placental tissues from high altitude (HA; n=14) and preeclamptic pregnancies (PE; n=5), relative to sea level controls (n=11). Lower panel, sFlt-1 protein densitometric analysis in HA, PE, and control SL. Data are normalized vs. sea-level samples. *P<0.05, PE and HA vs. SL. (D) Relative levels of circulating sFlt-1 protein in serum of pregnant patients from high altitude and lower altitude (control) near term (n=16). Values are mean (±SE). *P<0.05, HA vs. control.

FIG. 3 Immunolocalization of sFlt-1 in representative high altitude (HA), preeclampsia (PE) and sea level (SL) placental tissue. Panel a-c: Sea level controls, n=10. Panel d-f: High altitude samples, n=8. Panel g-k: early preeclampsia samples, n=6. Panel 1: Negative control (no 1° antibody). Brownish staining represents positive sFlt-1 immunostaining. (SK: syncytial knot, ET: endothelium, PV: perivascular.)

FIG. 4. Effect of low oxygen and hypoxia-reoxygenation (HR) on sFlt-1 expression in first trimester villous explants. (A) Expression of sFlt-1 mRNA in explants cultured at 3% and 8% vs. 20% O₂ measured by qRT-PCR analysis, n=7. *P<0.05, 3 and 8% vs. 20%. (B) Real-time RT-PCR analysis of sFlt-1 mRNA in explants cultured in 20% and 8% O₂ compared to HR conditions, n=5. *P<0.05, 8% vs. HR. (C) sFlt-1 protein concentration measured by ELISA in conditioned media from first trimester placental explants that were cultured in 20%, 8% and 3% O₂ compared to HR, n=8. *P<0.05, 3% vs. HR and 20%. Values are mean (±SE) of at least five separate experiments carried out in triplicate.

FIG. 5. Effect of DMOG and antisense oligonucleotides to HIF-1α on sFlt-1 expression in first trimester placental explants. (A) Effect of DMOG treatment on sFlt-1 transcript in villous explants assessed by qRT-PCR, n=3. *P<0.05, 3% and 20% DMOG vs. 20%. (B) Effect of DMOG treatment on spatial localization of sFlt-1 protein in villous explants, n=3. Brownish staining represents positive immunoreactivity. (C) TUNEL staining of villous explants and placental sample of early PE. Positive staining appears as dark brown nuclear staining. (D) Effect of antisense oligonucleotides to HIF-1α (AS) on sFlt-1 mRNA expression in explants, n=6. *P<0.05, 3% AS vs. 3%. (DMOG: Dimethyloxalyl-glycin). Values are mean (±SE) of at least three separate experiments carried out in triplicate.

FIG. 6. Mcl-1 Expression in Preeclamptic Pregnancies. Panel a: Quantitative real-time RT-PCR analysis of Mcl-1L and Mcl-1S transcripts in placental tissues from severe early-onset preeclampsia (PE: Black bar) and normotensive age-matched control tissues (AMC: Open bar). Panel b: Representative Mcl-1 immunoblot performed on AMC and PE total protein lysates. The classic long isoform of Mcl-1 is depicted. Mcl-1-specific bands around 29 kDa (referred to as p29) and 26 kDa (Mcl-1S) are also evident. Panel c: Densitometric analysis of Mcl-1-specific protein isoform bands between AMC (open bar; n=22) and PE (black bar; n=25). Panel d: Representative Mcl-1 immunoblot performed on total protein lysates from normal term placentae (term), term preeclamptic placentae (term PE) and normal term elective caesarian section placentae in absence of labour (C/S). Panel e. Representative Mcl-1 immunoblot performed on total protein lysates from placentae from severe early-onset preeclampsia, IUGR pregnancies, 35-37 weeks IUGR+PE pregnancies, pregnant patients with essential hypertension (EH) and normal term placentae (term). All immunoblots were confirmed for equal protein loading using ponceau staining. Data are presented as mean±SE of three or more separate experiments. *P<0.05, Student's t test.

FIG. 7. Inhibition of Caspase Activity and its effect on Mcl-1 Cleavage Panel a: Representative immunoblot of Mcl-1 isoforms in control (untreated explant) and explants exposed to H/R in presence of 100 μM concentration of pan-caspase inhibitor z-VAD-fmk dissolved in DMSO relative to control-treatment (DMSO alone). Panel b: Representative Mcl-1 immunoblot performed on protein lysates from explants exposed to H/R in presence of z-DEVD-fmk (in DMSO) and absence of caspase-3 inhibitor (DMSO alone) relative to untreated control tissue (Control). Panel c: Densitometric analysis of Mcl-1-specific protein isoform bands between H/R (open bar) and H/R+z-VAD-fmk (black bar) treated explants.

FIG. 8. Effect of Varying Oxygenation on Mcl-1 expression Panel a: Quantitative RT-PCR analyses of Mcl-1 isoforms L (black box) and S (white box) in first trimester villous explants exposed to 20% O₂, 3% O₂ and hypoxia/reoxygenation (H/R) conditions. Panel b: Representative immunoblot of Mcl-1 isoforms in first trimester villous explants exposed to 20% O₂, 3% O₂ and H/R. Panel c: Representative Mcl-1 immunoblot performed on total protein lysates obtained from normal first and second trimester placental tissues. All immunoblots were confirmed for equal protein loading using ponceau staining. Data are presented as mean±SE of three or more separate experiments. *P<0.05, Student's t test.

FIG. 9. Transcript Expression of Mcl-1 and Mtd Isoforms in Placental Tissue from SL, MA and HA Pregnancies. Panels a, b, c and d: Respectively, quantitative RT-PCR analysis of Mcl-1L (anti-apoptotic), Mcl-1S (pro-apoptotic), Mtd-L (pro-apoptotic) and Mtd-P (pro-apoptotic) transcript expression in sea-level (SL; n=10), moderate altitude (MA; n=10) and high altitude (HA; n=16) placentae. Data are presented as mean±SE. *P<0.05, Student's t test. Panels e and f: Respectively, Mcl-1 and Mtd immunoblots of protein lysates obtained from Sea Level (SL), Moderate Altitude (MA) and High Altitude (HA) placentae. All immunoblots were confirmed for equal protein loading using ponceau staining (not depicted). Panel g: Immunohistochemical localization of Mcl-1 (Top panels) and Mtd (Lower panels) in SL, MA and HA placentae. (S: stroma, ST: syncytium). Staining representative of 4 separate experiments carried out in triplicate.

FIG. 10. Markers of Cell Death in Conditions of Chronic and Pathological Placental Hypoxia. Panel a: Representative cleaved caspase-8 immunoblot in AMC and PE tissues. Panel b: Top: Representative immunoblot of cleaved caspase-3 performed on total placental protein lysates obtained from HA and control tissues (SL and MA). Bottom: Densitometric analysis of cleaved caspase-3 protein in HA (n=12) relative to control tissues (SL and MA, n=18). Panel c. Top: Representative immunoblot of cleaved caspase-8 performed on total placental protein lysates obtained from HA and control tissues (SL and MA). Bottom: Densitometric analysis of cleaved caspase-8 protein in HA (n=12) relative to control tissues (SL and MA, n=18). All immunoblots were confirmed for equal protein loading using ponceau staining. Data are presented as mean±SE of three separate experiments. *P<0.05, Student's t test.

FIG. 11. Syncytin Protein and Transcript Expression in Conditions of Chronic Placental Hypoxia (High Altitude Placentae). Panel a: Quantitative RT-PCR analysis of syncytin in SL (n=8), MA (n=10) and HA (n=10) placental tissues. Panel b: Quantitative RT-PCR analysis of cytokeratin 7 in HA (n=9) and control tissues (MA; n=9). Panel c: Representative immunoblot performed on total protein lysates from third trimester normal tissues with pre-immune serum (control) and serum of rabbits immunized with syncytin peptide (containing anti-syncytin polyclonal antibody). Panel d: Representative syncytin immunoblot performed on total protein lysate from HA and control (SL) placental tissues. Panel e: Representative syncytin immunoblot performed on total protein lysate from explants maintained under 20% and 3% oxygen. Panel f: Representative syncytin immunoblot performed on total protein lysates from first trimester and term placentae. Panel g. Quantitative RT-PCR analysis of syncytin in AMC (n=10) and PE (n=8) placental tissues. Panel h: Representative syncytin immunoblot performed on total placental protein lysates from AMC and PE tissues. Panel i: Densitometric syncytin protein analysis between AMC (n=13) and early-onset preeclamptic tissues (PE; n=13). All immunoblots were confirmed for equal protein loading using ponceau staining. Data are presented as mean±SE of three separate experiments. *P<0.05, Student's t test.

FIG. 12 is a schematic diagram showing the regulation of expression and activity of certain biomarkers in early onset severe preeclampsia.

FIG. 13 is a schematic diagram showing the regulation of expression and activity of certain biomarkers in early onset severe preeclampsia and intra-uterine growth restriction.

### DETAILED DESCRIPTION OF THE INVENTION

In accordance with the present invention there may be employed conventional molecular biology, microbiology, and recombinant DNA techniques within the skill of the art. Such techniques are explained fully in the literature. See for example, Sambrook, Fritsch, & Maniatis, Molecular Cloning: A Laboratory Manual, Second Edition (1989) Cold Spring Harbor Laboratory Press, Cold Spring Harbor, N.Y.); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

Unless defined otherwise, all technical and scientific terms used herein have the same meaning as commonly understood by one of ordinary skill in the art to which this invention belongs.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made.

### Diagnostic Methods

A variety of methods can be employed for the detection, diagnosis, monitoring, and prognosis of a condition requiring modulation of or involving trophoblast cell death, differentiation, invasion, and/or cell fusion and turnover, or status of such conditions, and for the identification of subjects with a predisposition to such conditions. Such methods may, for example, utilize one, two, three, four, five, or a plurality of Polynucleotide Markers, in particular, polynucleotides encoding one or more of a SMAD2, phospho-SMAD2, SMAD-3, phospho-SMAD3, SMAD7, or ceruloplasmin or fragments thereof, and binding agents (e.g. antibodies) against one, two, three, four, five, or a plurality of Polypeptide Markers, in particular, one or more of a SMAD2, phospho-SMAD2, SMAD-3, phospho-SMAD3, SMAD7, or ceruloplasmin, including peptide fragments. In particular, polynucleotides and antibodies may be used, for example, for (1) the detection of the presence of mutations in a one, two, three, four, five, or a plurality of Polynucleotide Markers, in particular polynucleotides encoding a SMAD2, SMAD-3, phospho-SMAD3, SMAD7, and/or ceruloplasmin or the detection of either an over- or under-expression of such polynucleotide mRNA relative to a normal state, or the qualitative or quantitative detection of alternatively spliced forms of such polynucleotide transcripts which may correlate with certain conditions or susceptibility toward a condition; and (2) the detection of either an over- or an under-abundance of one or more of Polypeptide Marker, in particular one or more of a SMAD2, phospho-SMAD2, SMAD-3, phospho-SMAD3, SMAD7, and/or ceruloplasmin relative to a normal state or a different stage of a condition, or the presence of a modified Polypeptide Marker, in particular, SMAD2, phospho-SMAD2, SMAD-3, phospho-SMAD3, SMAD7, and/or ceruloplasmin which correlates with a condition or state, or a progression toward a condition, or a particular type or stage of a condition.

The methods described herein can be adapted for diagnosing and monitoring a condition involving trophoblast cell death, differentiation, invasion, and/or cell fusion and turnover in biological samples from a subject. These applications require that the amount of a Polypeptide Marker in particular, SMAD2, phospho-SMAD2, SMAD-3, phospho-SMAD3, SMAD7, and/or ceruloplasmin, and/or polynucleotides encoding same, quantitated in a sample from a subject being tested be compared to a predetermined standard or cut-off value. The standard may correspond to levels quantitated for another sample or an earlier sample from the subject, or levels quantitated for a control sample. Levels for control samples from healthy subjects, different stages or types of condition, may be established by prospective and/or retrospective statistical studies. Healthy subjects who have no clinical evidence of a condition or abnormalities may be selected for statistical studies. Diagnosis may be made by a finding of altered levels, in particular statistically different levels of detected Markers, associated with a condition (e.g., preeclampsia) compared to a control sample or previous levels quantitated for the same subject. “Statistically different levels”, or “significant difference” in levels of markers in a patient sample compared to a control or standard may represent levels that are higher or lower than the standard error of the detection assay. In particular embodiments, the levels may be 1.5, 2, 3, 4, 5, or 6 times higher or lower than the control or standard.

In an aspect of the invention, a method is provided for diagnosing or monitoring in a subject a condition requiring modulation of or involving trophoblast cell death, differentiation, invasion, and/or cell fusion and turnover (e.g., preeclampsia), comprising detecting a Polypeptide Marker in particular, SMAD2, phospho-SMAD2, SMAD-3, phospho-SMAD3, SMAD7, and/or ceruloplasmin and/or Polynucleotide Markers, in particular polynucleotides encoding SMAD2, SMAD-3, phospho-SMAD3, SMAD7, and/or ceruloplasmin in a sample from the subject. In an embodiment of a diagnostic method of the invention, a method is provided for diagnosing increased risk of a condition requiring modulation of or involving trophoblast cell death, differentiation, invasion, and/or cell fusion and turnover (e.g., preeclampsia) in a subject comprising detecting Polynucleotide Markers, in particular polynucleotides encoding SMAD2, phospho-SMAD2, SMAD-3, phospho-SMAD3, SMAD7, and/or ceruloplasmin, in a sample from the subject.

In another aspect, the invention provides use of binding agents and/or polynucleotides that interact with Polypeptide Markers, in particular SMAD2, phospho-SMAD2, SMAD-3, phospho-SMAD3, SMAD7, and/or ceruloplasmin, or with Polynucleotide Markers in particular polynucleotides encoding a SMAD2, phospho-SMAD2, SMAD-3, phospho-SMAD3, SMAD7, and/or ceruloplasmin in the manufacture of a composition for detecting a condition requiring modulation of or involving trophoblast cell death, differentiation, invasion, and/or cell fusion and turnover (e.g., preeclampsia, choriocarcinoma, hydatiform mole, or molar pregnancy).

The methods described herein may be used to predict or evaluate the probability of a condition disclosed herein (e.g., preeclampsia), for example, in a sample freshly removed from a host. Such methods can be used to detect the condition (e.g., preeclampsia) and help in the diagnosis and prognosis of the condition. The methods can be used to detect the potential for the condition and to monitor a patient or a therapy.

The invention also contemplates a method for detecting a condition disclosed herein (e.g. a condition requiring modulation of or involving trophoblast cell death, differentiation, invasion, and/or cell fusion and turnover), or a predisposition to such condition, comprising producing a profile of levels of one or more Polypeptide Markers and comparing the profile with a reference to identify a profile for the patient indicative of the condition. In aspects, the Polypeptide Markers comprise or are selected from the group consisting of SMAD2, phospho-SMAD2, SMAD-3, phospho-SMAD3, SMAD7, ceruloplasmin, sFlt, Mtd-L, Mtd-S, Mtd-P, Mcl-1 isoforms (in particular Mcl-1S or Mcl-1L, or caspase cleaved Mcl-1S or Mcl-1L, in particular caspase cleaved Mcl-1L), TGFβ3, HIF1α, HIF-2α, endoglin, PHD1, PHD2, PHD3, VHL, Siah1/2, cullin 2, NEDD8, VEGF, FIH, syncytin, cleaved caspase (e.g., caspase-3), Fas, and/or p53. In an embodiment, the Polypeptide Markers comprise SMAD2, phospho-SMAD2, SMAD-3, phospho-SMAD3, SMAD7, and/or ceruloplasmin.

The invention further contemplates a method for analyzing a biological sample for the presence of one or more Polypeptide Markers, in particular a set of Polypeptide Markers comprising SMAD2, phospho-SMAD2, SMAD-3, phospho-SMAD3, SMAD7, and/or ceruloplasmin, and/or polynucleotides encoding same.

A method of the invention for diagnosing preeclampsia may comprise detecting or producing profiles of levels of a Mtd-S, Mtd-P or Mtd-L, (Soleymanlou N. et al, Cell Death Differ 12:441-452, 2005; Gene ID No. 51800; Accession No. NM_{—}032515; NP_{—}115904), sFlt (Accession number: U01134); SMAD2 (Gene ID 4087; NM_{—}001003652; NP_{—}001003652), SMAD3 (Gene ID No. 4088; NP_{—}005893; NM_{—}005902); SMAD7 (Gene ID No. 4092; NM_{—}005904; NP_{—}005895), ceruloplasmin (Gene ID. 1356; NM_{—}000096; NP_{—}000087), transforming growth factor β3 (TGFβ3) (e.g., Accession No. NP_{—}003230); transforming growth factor β1 (TGFβ1) (e.g., Accession No. NP_{—}000651); hypoxia-inducible factor 1, alpha subunit (HIF-1α) (e.g., Accession No. NP_{—}001521); hypoxia-inducible factor 1, beta subunit (HIF-1β) (e.g., Accession No. NP_{—}001659; NP_{—}848513; NP_{—}848514); hypoxia-inducible factor 2, alpha subunit (HIF-2α) (e.g., Accession No. Q99814); endoglin (Gene ID. No. 2022; NP_{—}000109; NM_{—}000118; UniProt No. P17813); von Hippel-Lindau tumor suppressor (VHL) (e.g., Accession Nos. NP_{—}000542 and NP_{—}937799); myeloid cell leukemia sequence 1 (Mcl-1) (e.g., Accession Nos. NP_{—}068779-isoform 1; NP_{—}877495-isoform 2; SEQ ID NO. 5, 6, or 9); prolyl-4-hydroxylase 1 (PHD1) (e.g., Accession No. NP_{—}071334); prolyl-4-hydroxylase 2 (PHD2) (e.g., Accession No. NP_{—}0600251; NP_{—}542770; and NP_{—}444274); prolyl-4-hydroxylase 3 (PHD3) (e.g., Accession No. NP_{—}071356); seven in absentia homolog 1 (Siah1) (e.g., Accession No. NP_{—}001006611 and NP_{—}003022); seven in absentia homolog 2 (Siah2) (e.g., Accession No. NP_{—}005058); vascular endothelial growth factor (VEGF) (e.g., Accession No. NP_{—}001020537 to NP_{—}001020541, NP 003367); syncytin (e.g., Accession No. NP_{—}055405); cullin 2 (e.g., Accession No. NP_{—}003582); neural precursor cell expressed, developmentally down-regulated 8 (NEDD) (e.g. Accession No. NP_{—}006147); factor inhibiting HIF (FIH) (e.g., Accession No. Q9NWT6); Fas (TNF receptor superfamily, member 6) (e.g., Accession No. NP_{—}000034; NP_{—}690610 through NP_{—}690616); cleaved caspase-3 (e.g., capase-3: Accession No. NP_{—}004337; NP_{—}116786; AAO25654); and tumor protein p53 (e.g., Accession No. NM_{—}000546); or, polynucleotides encoding these polypeptides. Exemplary nucleic acid sequences encoding the polypeptides are as follows: Mtd-P (NM_{—}016778), Mtd-L (NM_{—}016778), TGFβ3 (e.g. Accession No. NM_{—}003239, NM_{—}181054); TGFβ1 (e.g., Accession No. NM_{—}000660); endoglin (Gene ID. 2022, NM_{—}000118); HIF-1α (e.g., Accession No. NM_{—}001530, NP_{—}851397); SMAD2 (Gene ID. No. 4087; Accession Nos. NM_{—}001003652 and NM_{—}005901); SMAD3 (Gene ID No. 4058; Accession No. NM_{—}005902); SMAD7 (Gene ID No. 4092; Accession No. NM_{—}005904); ceruloplasmin (Gene ID No. 1356; Accession No. NM_{—}000096); VHL (e.g. Accession Nos. NM_{—}000551, NM_{—}198156); Mcl-1 (e.g., Accession Nos. NM_{—}021960-variant 1; NM_{—}182763-variant 2; SEQ ID NO. 7, 8, and 10); PHD1 (e.g., Accession No. NM_{—}022051); PHD2 (e.g., Accession Nos. NM_{—}053046, NM_{—}017555, NM_{—}080732); PHD3 (e.g., Accession No. NM_{—}022073); HIF-1β (e.g., Accession No. NM_{—}001668; NM_{—}178426; NM_{—}178427); seven in absentia homolog 1 (Siah1) (e.g., Accession Nos. NM_{—}001006610; NM_{—}0030311 and NM_{—}001006611); Siah2 (e.g., Accession No. NM_{—}005067); VEGF (e.g., Accession No. NM_{—}001025366 to NM_{—}001025370, NM_{—}003376), FIH-1 (e.g. Accession No. AF395830), syncytin (e.g., Accession No. NG_{—}004112), CUL2 (e.g., Accession No. NM_{—}003591); NEDD8 (e.g. Accession No. NM_{—}006156), Fas (e.g., Accession Nos. NM_{—}000043; NM_{—}152871; NM_{—}152872; NM_{—}152873 152877); cleaved caspase-3 (e.g., caspase-3: Accession No. NM_{—}004346; NM_{—}032991; AY219866); and, p53 (e.g., Accession No. NP_{—}000537).

It will be appreciated that the Polypeptide Markers disclosed herein include but are not limited to native-sequence polypeptides, isoforms, chimeric polypeptides, and all homologs, fragments, and precursors of the markers, including modified forms of the polypeptides and derivatives.

A “native-sequence polypeptide” comprises a polypeptide having the same amino acid sequence of a polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g. alternatively spliced forms or splice variants), and naturally occurring allelic variants.

The term “polypeptide variant” means a polypeptide having at least about 45%, 50%, 55%, 60%, 65%, 70%, 75%, 80%, 85%, 90%, 95%, 97%, 98%, or 99% amino acid sequence identity, particularly at least about 70-80%, more particularly at least about 85%, still more particularly at least about 90%, most particularly at least about 95% amino acid sequence identity with a native-sequence polypeptide. Such variants include, for instance, polypeptides wherein one or more amino acid residues are added to, or deleted from, the N- or C-terminus of the full-length or mature sequences of the polypeptide, including variants from other species, but excludes a native-sequence polypeptide. In aspects of the invention variants retain the immunogenic activity of the corresponding native-sequence polypeptide. An allelic variant may also be created by introducing substitutions, additions, or deletions into a polynucleotide encoding a native polypeptide sequence such that one or more amino acid substitutions, additions, or deletions are introduced into the encoded protein. Mutations may be introduced by standard methods, such as site-directed mutagenesis and PCR-mediated mutagenesis.

A “chimeric protein” or “fusion protein” comprises all or part (preferably biologically active) of a marker operably linked to a heterologous polypeptide (i.e., a polypeptide other than the marker). Within the fusion protein, the term “operably linked” is intended to indicate that a marker and the heterologous polypeptide are fused in-frame to each other. The heterologous polypeptide can be fused to the N-terminus or C-terminus of the marker. A useful fusion protein is a GST fusion protein in which a marker is fused to the C-terminus of GST sequences. Chimeric and fusion proteins can be produced by standard recombinant DNA techniques.

A modified form of a Polypeptide Marker referenced herein includes modified forms of the polypeptides and derivatives of the polypeptides, including post-translationally modified forms such as glycosylated, phosphorylated, acetylated, methylated or lapidated forms of the polypeptides.

A Polypeptide Marker disclosed herein may be prepared by recombinant or synthetic methods, or isolated from a variety of sources, or by any combination of these and similar techniques.

The invention provides a set of Polypeptide Markers correlated with trophoblast cell death, differentiation, invasion, and/or cell fusion or turnover, in particular early onset severe preeclampsia, late onset preeclampsia, or IUGR. A set of these markers that can be used for detection, diagnosis, prevention and therapy of conditions disclosed herein includes SMAD2, phospho-SMAD2, SMAD-3, phospho-SMAD3, SMAD7, ceruloplasmin, sFlt, Mtd-P, Mtd-L, Mtd-S, Mcl-1 isoforms (in particular Mcl-1S or Mcl-1L, or caspase cleaved Mcl-1S or Mcl-1L, in particular caspase cleaved Mcl-1L), TGFβ1, TGFβ3, HIF-1α, HIF-1β, HIF-2 α, VHL, cullin 2, NEDD8, PHD1, PHD2, PHD3, Siah1/2, cleaved caspase (e.g. caspase-3), syncytin, Fas, VEGF, FIH, and/or p53.

Thus, the invention provides marker sets that distinguish a condition requiring modulation of trophoblast cell death, differentiation, invasion, and/or cell fusion or turnover and uses therefor. In an aspect, the invention provides a method for classifying a condition requiring modulation of trophoblast cell death, differentiation, invasion, and/or cell fusion or turnover comprising detecting a difference in the expression of a plurality of markers in a sample from a patient relative to a control, the plurality of markers and/or polynucleotide markers comprising at least one, two, three, four, five, six, seven, eight, nine, or ten or more of SMAD2, phospho-SMAD2, SMAD-3, phospho-SMAD3, SMAD7, ceruloplasmin, sFlt, Mtd-P, Mtd-L, Mtd-S, Mcl-1 isoforms (in particular Mcl-1S or Mcl-1L, or caspase cleaved Mcl-1S or Mcl-1L, in particular caspase cleaved Mcl-1L), TGFβ3, HIF-1α, HIF-1β, HIF-2 α, VHL, cullin 2, NEDD8, PHD1, PHD2, PHD3, Siah1/2, cleaved caspase (e.g. caspase-3), syncytin, Fas, VEGF, FIH, and/or p53, and/or polynucleotides encoding same.

In an aspect the marker set comprises SMAD2, phospho-SMAD2, SMAD-3, phospho-SMAD3, SMAD7, ceruloplasmin, sFlt, Mtd-P and/or Mtd-L, and one or more of Mcl-1 isoforms (in particular Mcl-1S or Mcl-1L, or caspase cleaved Mcl-1S or Mcl-1L, in particular caspase cleaved Mcl-1L), TGFβ1, TGFβ3, HIF-1α, HIF-1β, HIF-2 α, VHL, cullin 2, NEDD8, PHD1, PHD2, PHD3, Siah1/2, cleaved caspase (e.g. caspase-3), syncytin, Fas, VEGF, FIH, and/or p53, and/or polynucleotides encoding same.

In another aspect the marker set comprises SMAD2, phospho-SMAD2, SMAD-3, phospho-SMAD3, SMAD7, ceruloplasmin, sFlt, Mtd-P, Mtd-L, Mcl-1L, Mcl-1c, TGFβ3, HIF-1α, VHL, PHD1, PHD2, PHD3, Siah1/2, and VEGF, and/or polynucleotides encoding same.

A control can comprise markers derived from a pool of samples from individual patients with no disease, or individuals with a known condition.

Samples that may be analyzed using the methods of the invention include those which are known or suspected to contain or express Markers associated with the conditions disclosed herein. The samples may be derived from a patient or a cell culture, and include but are not limited to biological fluids, tissue extracts, freshly harvested cells, and lysates of cells which have been incubated in cell cultures. Examples of samples include tissues (e.g. placenta), extracts, or cell cultures, including cells, cell lysates, conditioned medium from maternal cells, and physiological fluids, such as, for example, whole blood, plasma, serum, saliva, ocular lens fluid, cerebral spinal fluid, sweat, urine, milk, ascites fluid, amniotic fluid, vaginal fluid, synovial fluid, peritoneal fluid and the like.

A sample can be used directly as obtained from the source or following a pretreatment to modify the character of the sample. Therefore, a sample can be treated prior to use, such as preparing plasma from blood, diluting viscous fluids, and the like. Methods of treatment can involve filtration, distillation, extraction, concentration, inactivation of interfering components, the addition of reagents, and the like. Polypeptides and polynucleotides may be isolated from the samples and utilized in the methods of the invention.

In embodiments of the invention the sample is a mammalian sample, preferably human sample. In another embodiment the sample is a physiological fluid such as serum or tissue (e.g. placenta).

The samples that may be analyzed in accordance with the invention include polynucleotides from clinically relevant sources, preferably expressed RNA or a nucleic acid derived therefrom (cDNA or amplified RNA derived from cDNA that incorporates an RNA polymerase promoter). The target polynucleotides can comprise RNA, including, without limitation total cellular RNA, poly(A)⁺ messenger RNA (mRNA) or fraction thereof, cytoplasmic mRNA, or RNA transcribed from cDNA (i.e., cRNA; see, for example, Linsley & Schelter, U.S. patent application Ser. No. 09/411,074, or U.S. Pat. No. 5,545,522, 5,891,636, or 5,716,785). Methods for preparing total and poly(A)⁺ RNA are well known in the art, and are described generally, for example, in Sambrook et al., (1989, Molecular Cloning—A Laboratory Manual (2^{nd} Ed.), Vols. 1-3, Cold Spring Harbor Laboratory, Cold Spring Harbor, N.Y.) and Ausubel et al, eds. (1994, Current Protocols in Molecular Biology, vol. 2, Current Protocols Publishing, New York). RNA may be isolated from eukaryotic cells by procedures involving lysis of the cells and denaturation of the proteins contained in the cells. Additional steps may be utilized to remove DNA. Cell lysis may be achieved with a nonionic detergent, followed by microcentrifugation to remove the nuclei and hence the bulk of the cellular DNA. (See Chirgwin et al., 1979, Biochemistry 18:5294-5299). Poly(A)+RNA can be selected using oligo-dT cellulose (see Sambrook et al., 1989, Molecular Cloning—A Laboratory Manual (2nd Ed.), Vols. 1-3, Cold Spring Harbor Laboratory, Cold Spring Harbor, N.Y.). In the alternative, RNA can be separated from DNA by organic extraction, for example, with hot phenol or phenol/chloroform/isoamyl alcohol.

It may be desirable to enrich mRNA with respect to other cellular RNAs, such as transfer RNA (tRNA) and ribosomal RNA (rRNA). Most mRNAs contain a poly(A) tail at their 3′ end allowing them to be enriched by affinity chromatography, for example, using oligo(dT) or poly(U) coupled to a solid support, such as cellulose or Sephadex™ (see Ausubel et al., eds., 1994, Current Protocols in Molecular Biology, vol. 2, Current Protocols Publishing, New York). Bound poly(A)+ mRNA is eluted from the affinity column using 2 mM EDTA/0.1% SDS.

A sample of RNA can comprise a plurality of different mRNA molecules each with a different nucleotide sequence. In an aspect of the invention, the mRNA molecules in the RNA sample comprise at least 100 different nucleotide sequences.

### Polynucleotide Methods

A condition requiring regulation of trophoblast cell death, differentiation, invasion, and/or cell fusion and turnover (e.g., preeclampsia), or stage or type of same, may be detected based on the level in a sample of Polynucleotide Markers, in particular polynucleotides encoding SMAD2, SMAD-3, phospho-SMAD3, SMAD7, and/or ceruloplasmin. Techniques for detecting polynucleotides such as polymerase chain reaction (PCR) and hybridization assays are well known in the art.

Probes may be used in hybridization techniques to detect genes that encode a Polypeptide Marker disclosed herein. The technique generally involves contacting and incubating polynucleotides (e.g. recombinant DNA molecules, cloned genes) obtained from a sample from a patient or other cellular source with a probe under conditions favourable for the specific annealing of the probes to complementary sequences in the polynucleotides. After incubation, the non-annealed nucleic acids are removed, and the presence of polynucleotides that have hybridized to the probe if any are detected.

Nucleotide probes for use in the detection of nucleic acid sequences in samples may be constructed using conventional methods known in the art. Suitable probes may be based on nucleic acid sequences encoding at least 5 sequential amino acids from regions of a Polynucleotide Marker, preferably they comprise 10-30, 10-40, 15-40, 20-50, 40-80, 50-150, or 80-120 nucleotides.

A nucleotide probe may be labelled with a detectable substance such as a radioactive label which provides for an adequate signal and has sufficient half-life such as ³²P, ³H, ¹⁴C or the like. Other detectable substances which may be used include antigens that are recognized by a specific labelled antibody, fluorescent compounds, enzymes, antibodies specific for a labelled antigen, and luminescent compounds. An appropriate label may be selected having regard to the rate of hybridization and binding of the probe to the nucleotide to be detected and the amount of nucleotide available for hybridization. Labelled probes may be hybridized to nucleic acids on solid supports such as nitrocellulose filters or nylon membranes as generally described in Sambrook et al, 1989, Molecular Cloning, A Laboratory Manual (2nd ed.).

The nucleic acid probes may be used to detect genes, preferably in human cells, that encode Polynucleotide Markers. The nucleotide probes may also be useful in the diagnosis of conditions requiring regulation of trophoblast cell death, differentiation, invasion, and/or cell fusion and turnover (e.g., preeclampsia); in monitoring the progression of such disorders or conditions; or monitoring a therapeutic treatment. In aspects of the invention the nucleotide probes are useful in the diagnosis, prediction, management and control of preeclampsia involving one or more Polynucleotide Marker disclosed herein, in monitoring the progression of preeclampsia; or monitoring a therapeutic treatment.

The levels of mRNA or polynucleotides derived therefrom can be determined using hybridization methods known in the art. For example, RNA can be isolated from a sample and separated on a gel. The separated RNA can then be transferred to a solid support and nucleic acid probes representing one or more markers can be hybridized to the solid support and the amount of marker-derived RNA can be determined. Such determination can be visual or machine-aided (e.g. use of a densitometer). Dot-blot or slot-blot may also be used to determine RNA. RNA or nucleic acids derived therefrom from a sample are labeled, and then hybridized to a solid support containing oligonucleotides derived from one or more marker genes that are placed on the solid support at discrete, easily-identifiable locations. Hybridization or the lack thereof, of the labeled RNA to the solid support oligonucleotides is determined visually or by densitometer.

The detection of Polynucleotide Markers may involve the amplification of specific gene sequences using an amplification method such as polymerase chain reaction (PCR), followed by the analysis of the amplified molecules using techniques known to those skilled in the art. Suitable primers can be routinely designed by one of skill in the art. By way of example, at least two oligonucleotide primers may be employed in a PCR based assay to amplify a portion of a polynucleotide(s) derived from a sample, wherein at least one of the oligonucleotide primers is specific for (i.e. hybridizes to) a Polynucleotide Marker. The amplified cDNA is then separated and detected using techniques well known in the art, such as gel electrophoresis.

In order to maximize hybridization under assay conditions, primers and probes employed in the methods of the invention generally have at least about 60%, preferably at least about 75%, and more preferably at least about 90% identity to a portion of a Polynucleotide Marker; that is, they are at least 10 nucleotides, and preferably at least 20 nucleotides in length. In an embodiment the primers and probes are at least about 10-40 nucleotides in length.

Hybridization and amplification techniques described herein may be used to assay qualitative and quantitative aspects of polynucleotide expression. For example, RNA may be isolated from a cell type or tissue known to express a Polynucleotide Marker and tested utilizing the hybridization (e.g. standard Northern analyses) or PCR techniques referred to herein. The techniques may be used to detect differences in transcript size which may be due to normal or abnormal alternative splicing. The techniques may be used to detect quantitative differences between levels of full length and/or alternatively splice transcripts detected in normal individuals relative to those individuals exhibiting symptoms of a condition requiring regulation of trophoblast cell death, differentiation, invasion, and/or cell fusion and turnover (e.g., preeclampsia).

In an aspect of the invention, a method is provided employing reverse transcriptase-polymerase chain reaction (RT-PCR), in which PCR is applied in combination with reverse transcription. Generally, RNA is extracted from a sample using standard techniques (for example, guanidine isothiocyanate extraction as described by Chomcynski and Sacchi, Anal. Biochem. 162:156-159, 1987) and is reverse transcribed to produce cDNA. The cDNA is used as a template for a polymerase chain reaction. The cDNA is hybridized to a set of primers, at least one of which is specifically designed against polynucleotide sequence of a marker disclosed herein. Once the primer and template have annealed a DNA polymerase is employed to extend from the primer, to synthesize a copy of the template. The DNA strands are denatured, and the procedure is repeated many times until sufficient DNA is generated to allow visualization by ethidium bromide staining and agarose gel electrophoresis.

In an embodiment, the invention provides a method wherein polynucleotides that are mRNA are detected by (a) isolating mRNA from a sample and combining the mRNA with reagents to convert it to cDNA; (b) treating the converted cDNA with amplification reaction reagents and nucleic acid primers that hybridize to a Polynucleotide Marker, to produce amplification products; (d) analyzing the amplification products to detect an amount of mRNA; and (e) comparing the amount of mRNA to an amount detected against a panel of expected values for normal subjects derived using similar nucleic acid primers.

Positive samples containing a Polynucleotide Marker in particular higher levels in patient samples compared to controls (e.g. normal sample) may be indicative of a condition, (e.g., preeclampsia), and/or that the patient is not responsive to or tolerant of a therapy. Alternatively, negative samples or lower levels compared to a control (e.g. normal samples or negative samples) may also be indicative of a condition, and/or that a patient is not responsive to or tolerant of a therapy.

Amplification may be performed on samples obtained from a subject with a suspected condition described herein (e.g. suspected preeclampsia) and an individual who is not predisposed to such condition. The reaction may be performed on several dilutions of cDNA spanning at least two orders of magnitude. A significant difference in expression in several dilutions of the subject sample as compared to the same dilutions of the normal sample may be considered positive for the presence of the condition (e.g. preeclampsia).

Genotyping techniques known to one skilled in the art can be used to type polymorphisms that are in close proximity to the mutations in a gene encoding a Polypeptide Marker. The polymorphisms may be used to identify individuals in families that are likely to carry mutations. If a polymorphism exhibits linkage disequalibrium with mutations in a Polynucleotide Marker, it can also be used to screen for individuals in the general population likely to carry mutations. Polymorphisms which may be used include restriction fragment length polymorphisms (RFLPs), single-base polymorphisms, and simple sequence repeat polymorphisms (SSLPs). A probe of the invention may be used to directly identify RFLPs. A probe or primer of the invention can additionally be used to isolate genomic clones such as YACs, BACs, PACs, cosmids, phage or plasmids. The DNA in the clones can be screened for SSLPs using hybridization or sequencing procedures.

The primers and probes may be used in the above-described methods in situ i.e. directly on tissue sections (fixed and/or frozen) of patient tissue obtained from biopsies or resections.

Oligonucleotides or longer fragments derived from a Polynucleotide Marker may be used as targets in a micro-array. The micro-array can be used to simultaneously monitor the expression levels of Polynucleotide Marker(s). The micro-array can also be used to identify genetic variants, mutations, and polymorphisms. The information from the micro-array may be used to determine gene function, to understand the genetic basis of a condition (e.g. preeclampsia), to diagnose a condition (e.g. preeclampsia), and to develop and monitor the activities of therapeutic agents. Thus, the invention also includes an array comprising one or more Polynucleotide Marker or a Marker set described herein. The array can be used to assay expression of polynucleotides in the array. The invention allows the quantitation of expression of one or more polynucleotides. Arrays are also useful for ascertaining differential expression patterns of the polynucleotides as described herein, and optionally other markers, in normal and abnormal samples. This may provide a battery of nucleic acids that could serve as molecular targets for diagnosis or therapeutic intervention.

The preparation, use, and analysis of micro-arrays are well known to a person skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO95/251116; Shalon, D. et al. (1995) PCT application WO95/35505; Heller, R. A. et al. (1997) Proc. Natl. Acad. Sci. 94:2150-2155; and Heller, M. J. et al. (1997) U.S. Pat. No. 5,605,662.). A variety of arrays are made in research and manufacturing facilities worldwide, some of which are available commercially. By way of example, spotted arrays and in situ synthesized arrays are two kinds of nucleic acid arrays that differ in the manner in which the nucleic acid materials are placed onto the array substrate. A widely used in situ synthesized oligonucleotide array is GeneChip™ made by Affymetrix, Inc. Examples of spotted cDNA arrays include LifeArray made by Incyte Genomics and DermArray made by IntegriDerm (or Invitrogen). Pre-synthesized and amplified cDNA sequences are attached to the substrate of spotted arrays. Protein and peptide arrays also are known [(see for example, Zhu et al., Science 293:2101 (2001)].

Thus, the invention also includes an array comprising one or more Polynucleotide Markers associated with trophoblast cell death, differentiation, invasion, and/or cell fusion and turnover including without limitation SMAD2, phospho-SMAD2, SMAD-3, phospho-SMAD3, SMAD7, ceruloplasmin, sFlt, Mtd-P, Mtd-L, Mtd-S, Mcl-1 isoforms (in particular Mcl-1S or Mcl-1L, or caspase cleaved Mcl-1S or Mcl-1L, in particular caspase cleaved Mcl-1L), TGFβ3, HIF-1α, HIF-2α, HIF-1β, VHL, PHD1, PHD2, PHD3, Siah1/2, VEGF, FIH, syncytin, cleaved caspase (e.g. caspase-3), cullin 2, NEDD8, Fas, and/or p53. The array can be used to assay expression of the markers in the array. The invention allows the quantitation of expression of one or more markers.

The invention provides microarrays comprising a disclosed marker set. In one embodiment, the invention provides a microarray for distinguishing conditions requiring modulation of trophoblast cell death, differentiation, invasion, and/or cell fusion and turnover comprising a positionally-addressable array of polynucleotide probes bound to a support, the polynucleotide probes comprising a plurality of polynucleotide probes of different nucleotide sequences, each of the different nucleotide sequences comprising a sequence complementary and hybridizable to a plurality of genes, the plurality comprising or consisting essentially of at least 2, 3, 4, 5, 6, 7, 8, 9, 10, 11, 12, 13, 14, 15, 16, 17, 18, 19, 20, or 25, Polynucleotide Markers, in particular genes corresponding to the markers SMAD2, SMAD-3, SMAD7, ceruloplasmin, sFlt, Mtd-P, Mtd-L, Mtd-S, Mcl-1 isoforms (in particular Mcl-1S or Mcl-1L, or caspase cleaved Mcl-1S or Mcl-1L, in particular caspase cleaved Mcl-1L), TGFβ3, TGFβ1, HIF-1α, HIF-1β, HIF-2α, VHL, cleaved caspase (e.g. caspase-3), PHD1, PHD2, PHD3, Siah1/2, syncytin, VEGF, FIH, cullin 2, NEDD8, Fas, and/or p53. An aspect of the invention provides microarrays comprise at least 5, 10, 15, 20, or 25 of the Polynucleotide Markers or a set of Markers.

The invention provides gene marker sets that distinguish conditions disclosed herein and uses therefor. In an aspect, the invention provides a method for classifying a condition disclosed herein comprising detecting a difference in the expression of a plurality of genes in a sample from a subject relative to a control, the plurality of genes consisting of at least 2, 5, 6, 7, 8, 9, 10, 15, 20, or 25 Polynucleotide Markers, in particular at least 3, 4, 5, 10, 15, or 20 of the genes encoding the markers SMAD2, SMAD-3, phospho-SMAD3, SMAD7, ceruloplasmin, sFlt, Mtd-P, Mtd-L, Mtd-S, Mcl-1 isoforms (in particular Mcl-1S or Mcl-1L, or caspase cleaved Mcl-1S or Mcl-1L, in particular caspase cleaved Mcl-1L), TGFβ3, TGFβ1, HIF-1α, HIF-2α, HIF-1β, endoglin, VHL, cleaved caspase (e.g. caspase-3), PHD1, PHD2, PHD3, Siah1/2, syncytin, VEGF, FIH, cullin 2, NEDD8, Fas, and/or p53. In specific aspects, the plurality of genes consists of at least 10 or 15 of the genes encoding the markers SMAD2, SMAD-3, phospho-SMAD3, SMAD7, ceruloplasmin, or sFlt, Mtd-P, Mtd-L, Mtd-S, Mcl-1 isoforms (in particular Mcl-1S, Mcl-1 c or Mcl-1L, or caspase cleaved Mcl-1S or Mcl-1L, in particular caspase cleaved Mcl-1L), TGFβ3, TGFβ1, HIF-1α, HIF-2α, HIF-1β, endoglin, VHL, cleaved caspase (e.g. caspase-3), PHD1, PHD2, PHD3, Siah1/2, syncytin, VEGF, FIH, cullin 2, NEDD8, Fas, and/or p53. In another specific aspect, the control comprises nucleic acids derived from a pool of samples from individual control patients.

The invention provides a method for classifying a condition disclosed herein by calculating the similarity between the expression of at least 5, 10, 15, 20, or 25, Polynucleotide Markers, in particular polynucleotides encoding markers comprising or selected from the group consisting of SMAD2, phospho-SMAD2, phospho-SMAD2, SMAD-3, phospho-SMAD3, SMAD7, ceruloplasmin, sFlt, Mtd-P, Mtd-L, Mtd-S, Mcl-1 isoforms (in particular Mcl-1S, Mcl-1c, or Mcl-1L, or caspase cleaved Mcl-1S or Mcl-1L, in particular caspase cleaved Mcl-1L), TGFβ3, TGFβ1, HIF-1α, HIF-2α, HIF-1β, endoglin, VHL, cleaved caspase (e.g. caspase-3), PHD1, PHD2, PHD3, Siah1/2, syncytin, VEGF, FIH, cullin 2, NEDD8, Fas, and/or p53, in a sample to the expression of the same markers in a control pool.

In an aspect, the invention provides a method for classifying a condition disclosed herein by calculating the similarity between the expression of at least 5, 10, 15, 20 or 25 Polynucleotide Markers in particular polynucleotides encoding markers comprising or selected from the group consisting of SMAD2, phospho-SMAD2, SMAD-3, phospho-SMAD3, SMAD7, ceruloplasmin, sFlt, Mtd-P, Mtd-L, Mtd-S, Mcl-1 isoforms (in particular Mcl-1S, Mcl-1c, or Mcl-1L, or caspase cleaved Mcl-1S or Mcl-1L, in particular caspase cleaved Mcl-1L), TGFβ3, TGFβ1, HIF-1α, HIF-2α, HIF-1β, endoglin, VHL, cleaved caspase (e.g. caspase-3), PHD1, PHD2, PHD3, Siah1/2, syncytin, VEGF, FIH, cullin 2, NEDD8, Fas, and/or p53 in a sample to the expression of the same markers in a control pool, comprising the steps of:
(a) labeling nucleic acids derived from a sample, with a fluorophore to obtain a first pool of fluorophore-labeled nucleic acids;
(b) labeling with a second fluorophore a first pool of nucleic acids derived from two or more disease samples, and a second pool of nucleic acids derived from two or more control samples;
(c) contacting the first fluorophore-labeled nucleic acid and the first pool of second fluorophore-labeled nucleic acid with a first microarray under conditions such that hybridization can occur, and contacting the first fluorophore-labeled nucleic acid and the second pool of second fluorophore-labeled nucleic acid with a second microarray under conditions such that hybridization can occur, detecting at each of a plurality of discrete loci on the first microarray a first fluorescent emission signal from the first fluorophore-labeled nucleic acid and a second fluorescent emission signal from the first pool of second fluorophore-labeled genetic matter that is bound to the first microarray and detecting at each of the marker loci on the second microarray the first fluorescent emission signal from the first fluorophore-labeled nucleic acid and a third fluorescent emission signal from the second pool of second fluorophore-labeled nucleic acid;
(d) determining the similarity of the sample to patient and control pools by comparing the first fluorescence emission signals and the second fluorescence emission signals, and the first emission signals and the third fluorescence emission signals; and
(e) classifying the sample as from a individual with a condition where the first fluorescence emission signals are more similar to the second fluorescence emission signals than to the third fluorescent emission signals, and classifying the sample as from an individual without the condition where the first fluorescence emission signals are more similar to the third fluorescence emission signals than to the second fluorescent emission signals, wherein the first microarray and the second microarray are similar to each other, exact replicas of each other, or are identical, and wherein the similarity is defined by a statistical method such that the cell sample and control are similar where the p value of the similarity is less than 0.01.

In an embodiment, the array can be used to monitor the time course of expression of one or more markers in the array. This can occur in various biological contexts such as disease progression. The array is also useful for ascertaining differential expression patterns of markers, and optionally other markers, in normal and abnormal cells. This may provide a battery of nucleic acids that could serve as molecular targets for diagnosis or therapeutic intervention.

Microarrays typically contain at separate sites nanomolar quantities of individual genes, cDNAs, or ESTs on a substrate (e.g. nitrocellulose or silicon plate, or photolithographically prepared glass substrate). The arrays are hybridized to cDNA probes using conventional techniques with gene-specific primer mixes. The target polynucleotides to be analyzed are isolated, amplified and labeled, typically with fluorescent labels, radiolabels or phosphorous label probes. After hybridization is completed, the array is inserted into the scanner, where patterns of hybridization are detected. Data are collected as light emitted from the labels incorporated into the target, which becomes bound to the probe array. Probes that completely match the target generally produce stronger signals than those that have mismatches. The sequence and position of each probe on the array are known, and thus by complementarity, the identity of the target nucleic acid applied to the probe array can be determined.

Microarrays can be prepared by selecting polynucleotide probes and immobilizing them to a solid support or surface. The probes may comprise DNA sequences, RNA sequences, copolymer sequences of DNA and RNA, DNA and/or RNA analogues, or combinations thereof. The probe sequences may be full or partial fragments of genomic DNA, or they may be synthetic oligonucleotide sequences synthesized either enzymatically in vivo, enzymatically in vitro (e.g., by PCR), or non-enzymatically in vitro.

The probe or probes used in the methods of the invention can be immobilized to a solid support or surface which may be either porous or non-porous. For example, the probes can be attached to a nitrocellulose or nylon membrane or filter covalently at either the 3′ or the 5′ end of the polynucleotide probe. The solid support may be a glass or plastic surface. In an aspect of the invention, hybridization levels are measured to microarrays of probes consisting of a solid support on the surface of which are immobilized a population of polynucleotides, such as a population of DNA or DNA mimics, or, alternatively, a population of RNA or RNA mimics. A solid support may be a nonporous or, optionally, a porous material such as a gel.

In accordance with embodiments of the invention, a microarray is provided comprising a support or surface with an ordered array of hybridization sites or “probes” each representing one of the markers described herein. The microarrays can be addressable arrays, and in particular positionally addressable arrays. Each probe of the array is typically located at a known, predetermined position on the solid support such that the identity of each probe can be determined from its position in the array. In particular embodiments, each probe is covalently attached to the solid support at a single site.

Microarrays used in the present invention are preferably (a) reproducible, allowing multiple copies of a given array to be produced and easily compared with each other; (b) made from materials that are stable under hybridization conditions; (c) small, (e.g., between 1 cm² and 25 cm², between 12 cm² and 13 cm², or 3 cm²; and (d) comprise a unique set of binding sites that will specifically hybridize to the product of a single gene in a cell (e.g., to a specific mRNA, or to a specific cDNA derived therefrom). However, it will be appreciated that larger arrays may be used particularly in screening arrays, and other related or similar sequences will cross hybridize to a given binding site.

In accordance with an aspect of the invention, the microarray is an array in which each position represents one of the markers described herein. Each position of the array can comprise a DNA or DNA analogue based on genomic DNA to which a particular RNA or cDNA transcribed from a genetic marker can specifically hybridize. A DNA or DNA analogue can be a synthetic oligomer or a gene fragment. In an embodiment, probes representing each of the Markers are present on the array. In a preferred embodiment, the array comprises at least 5, 10, 15, 20, or 25 of the Markers.

Probes for the microarray can be synthesized using N-phosphonate or phosphoramidite chemistries (Froehler et al., 1986, Nucleic Acid Res. 14:5399-5407; McBride et al., 1983, Tetrahedron Lett. 24:246-248). Synthetic sequences are typically between about 10 and about 500 bases, 20-100 bases, or 40-70 bases in length. Synthetic nucleic acid probes can include non-natural bases, such as, without limitation, inosine. Nucleic acid analogues such as peptide nucleic acid may be used as binding sites for hybridization. (see, e.g., Egholm et al., 1993, Nature 363:566-568; U.S. Pat. No. 5,539,083).

Probes can be selected using an algorithm that takes into account binding energies, base composition, sequence complexity, cross-hybridization binding energies, and secondary structure (see Friend et al., International Patent Publication WO 01/05935, published Jan. 25, 2001).

Positive control probes, (e.g., probes known to be complementary and hybridize to sequences in the target polynucleotides), and negative control probes, (e.g., probes known to not be complementary and hybridize to sequences in the target polynucleotides) are typically included on the array. Positive controls can be synthesized along the perimeter of the array or synthesized in diagonal stripes across the array. A reverse complement for each probe can be next to the position of the probe to serve as a negative control.

The probes can be attached to a solid support or surface, which may be made from glass, plastic (e.g., polypropylene, nylon), polyacrylamide, nitrocellulose, gel, or other porous or nonporous material. The probes can be printed on surfaces such as glass plates (see Schena et al., 1995, Science 270:467-470). This method may be particularly useful for preparing microarrays of cDNA (See also, DeRisi et al., 1996, Nature Genetics 14:457-460; Shalon et al., 1996, Genome Res. 6:639-645; and Schena et al., 1995, Proc. Natl. Acad. Sci. U.S.A. 93:10539-11286).

High-density oligonucleotide arrays containing thousands of oligonucleotides complementary to defined sequences, at defined locations on a surface can be produced using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). Using these methods oligonucleotides (e.g., 60-mers) of known sequence are synthesized directly on a surface such as a derivatized glass slide. The array produced may be redundant, with several oligonucleotide molecules per RNA.

Microarrays can be made by other methods including masking (Maskos and Southern, 1992, Nuc. Acids. Res. 20:1679-1684). In an embodiment, microarrays of the present invention are produced by synthesizing polynucleotide probes on a support wherein the nucleotide probes are attached to the support covalently at either the 3′ or the 5′ end of the polynucleotide.

### Polypeptide Methods

Polypeptide Markers in particular SMAD2, phospho-SMAD2, SMAD-3, phospho-SMAD3, SMAD7, and ceruloplasmin, may be detected using a binding agent. “Binding agent” refers to a substance such as a polypeptide or antibody that specifically binds to one or more Polypeptide Marker. A substance “specifically binds” to one or more Polypeptide Marker if is reacts at a detectable level with one or more polypeptide, and does not react detectably with peptides containing an unrelated or different sequence. Binding properties may be assessed using an ELISA, which may be readily performed by those skilled in the art (see for example, Newton et al, Develop. Dynamics 197: 1-13, 1993). A binding agent may be a ribosome, with or without a peptide component, an aptamer, an RNA molecule, or a polypeptide. A binding agent may be a polypeptide that comprises one or more polypeptide sequence, a peptide variant thereof, or a non-peptide mimetic of such a sequence. An aptamer includes a DNA or RNA molecule that binds to nucleic acids and proteins. An aptamer that binds to a protein (or binding domain) or a polynucleotide can be produced using conventional techniques, without undue experimentation. [For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996)].

Binding agents may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, 1988). In general, the presence or absence of Polypeptide Markers in a subject may be determined by (a) contacting a sample from the subject with binding agents that interacts with Polypeptide Markers; (b) detecting in the sample levels of the Polypeptide Markers or complexes that bind to the binding agents; and (c) comparing the levels of Polypeptide Markers with predetermined standard or cut-off values.

In the context of certain methods of the invention, a sample, binding agents (e.g. antibodies specific for one or more Polypeptide Marker), may be immobilized on a carrier or support. For example, an antibody or sample may be immobilized on a carrier or solid support which is capable of immobilizing cells, antibodies etc. Suitable carriers or supports may comprise nitrocellulose, or glass, polyacrylamides, gabbros, and magnetite. The support material may have any possible configuration including spherical (e.g. bead), cylindrical (e.g. inside surface of a test tube or well, or the external surface of a rod), or flat (e.g. sheet, test strip). Immobilization typically entails separating the binding agent from any free analytes (e.g. free Polypeptide Marker or free complexes thereof) in the reaction mixture.

Binding agents may be labeled using conventional methods with a detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol, enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy. Where a radioactive label is used as a detectable substance, a polypeptide may be localized by radioautography. The results of radioautography may be quantitated by determining the density of particles in the radioautographs by various optical methods, or by counting the grains.

Binding agents, including antibodies to Polypeptide Markers or protein complexes comprising Polypeptide Markers, or peptides that interact with Polypeptide Markers or complexes thereof, may also be indirectly labeled with a ligand binding partner. For example, the antibodies, or peptides may be conjugated to one partner of a ligand binding pair, and the polypeptide may be coupled to the other partner of the ligand binding pair. Representative examples include avidin-biotin, and riboflavin-riboflavin binding protein. In an embodiment the binding agents (e.g. antibodies) are biotinylated. Methods for conjugating binding agents such as antibodies with a ligand binding partner may be readily accomplished by one of ordinary skill in the art (see Wilchek and Bayer, “The Avidin-Biotin Complex in Bioanalytical Applications,” Anal. Biochem. 171:1-32, 1988).

Binding agents can directly or indirectly interact with Polypeptide Markers. Indirect methods may be employed in which a primary binding agent-binding partner interaction is amplified by introducing a second agent. For example, a primary polypeptide-antibody reaction may be amplified by the introduction of a second antibody, having specificity for the antibody reactive against the primary polypeptide. By way of example, if the antibody having specificity against a Polypeptide Marker is a rabbit IgG antibody, the second antibody may be goat anti-rabbit gamma-globulin labeled with a detectable substance as described herein.

The presence of Polypeptide Markers may be determined by measuring the binding of the Polypeptide Markers to molecules (or parts thereof) which are known to interact with the polypeptide. In aspects of the invention, peptides derived from sites on a polypeptide which binds to a polypeptide may be used. A peptide derived from a specific site on a binding polypeptide may encompass the amino acid sequence of a naturally occurring binding site, any portion of that binding site, or other molecular entity that functions to bind an associated molecule. A peptide derived from such a site will interact directly or indirectly with an associated molecule in such a way as to mimic the native binding site. Such peptides may include competitive inhibitors, enhancers, peptide mimetics, and the like as discussed herein.

In aspects of the invention, the binding agent is an antibody. Antibodies specifically reactive with Polypeptide Markers, or derivatives, such as enzyme conjugates or labelled derivatives, may be used to detect the polypeptides in various samples (e.g. biological materials). They may be used as diagnostic or prognostic reagents and they may be used to detect abnormalities in the level of polypeptide expression, or abnormalities in the structure, and/or temporal, tissue, cellular, or subcellular location of a polypeptide. Antibodies may also be used to screen potentially therapeutic compounds in vitro to determine their effects on conditions disclosed herein (e.g., preeclampsia). In vitro immunoassays may also be used to assess or monitor the efficacy of particular therapies. The antibodies of the invention may also be used in vitro to determine the level of polynucleotide expression in cells genetically engineered to produce a polypeptide.

In particular the invention provides a diagnostic method for monitoring or diagnosing a condition disclosed herein in a subject by quantitating Polypeptide Markers or complexes thereof in a biological sample from the subject comprising reacting the sample with antibodies specific for Polypeptide Markers or complexes thereof, which are directly or indirectly labeled with detectable substances and detecting the detectable substances. In a particular embodiment of the invention, a SMAD2, phospho-SMAD2, SMAD-3, phospho-SMAD3, SMAD7, and/or ceruloplasmin are quantitated or measured.

In an aspect of the invention, a method for detecting a condition disclosed herein is provided comprising:
(a) obtaining a sample suspected of containing Polypeptide Markers or complexes thereof;
(b) contacting the sample with antibodies that specifically bind to Polypeptide Markers or complexes thereof under conditions effective to bind the antibodies and form complexes;
(c) measuring the amount of Polypeptide Markers or complexes thereof present in the sample by quantitating the amount of the antibody-polypeptide complexes; and
(d) comparing the amount of Polypeptide Markers or complexes thereof present in the samples with the amount of Polypeptide Markers or complexes thereof in a control, wherein a change or significant difference in the amount of Polypeptide Markers or complexes thereof in the sample compared with the amount in the control is indicative of the condition.

The amount of antibody complexes may also be compared to a value representative of the amount of antibody complexes from an individual not at risk of, or afflicted with, a condition or having a condition at different stages. A significant difference in antibody complex formation may be indicative of an advanced condition, or an unfavourable prognosis.

In embodiments of the methods of the invention, Polypeptide Markers or complexes thereof are detected in samples and higher levels, in particular significantly higher levels compared to a control (e.g. normal) is indicative of a condition (e.g. preeclampsia).

In an embodiment, the invention contemplates a method for monitoring the progression of a condition disclosed herein in an individual, comprising:
(a) contacting antibodies which bind to Polypeptide Markers or complexes thereof with a sample from the individual so as to form complexes comprising the antibodies and Polypeptide Markers or complexes thereof in the sample;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of a condition, condition stage, and/or progression of the condition in the individual.

In methods of the invention the step of contacting a sample with a binding agent (e.g. antibodies) may be accomplished by any suitable technique so that detection can occur. In particular, antibodies may be used in any known immunoassays that rely on the binding interaction between antigenic determinants of one or more Polypeptide Marker or complexes thereof and the antibodies. Immunoassay procedures for in vitro detection of antigens in fluid samples are well known in the art, as well as widely established and used in the commercial diagnostic industry. [See for example, Paterson et al., Int. J. Can. 37:659 (1986) and Burchell et al., Int. J. Can. 34:763 (1984) for a general description of immunoassay procedures]. Qualitative and/or quantitative determinations of Polypeptide Markers or complexes thereof in a sample may be accomplished by competitive or non-competitive immunoassay procedures in either a direct or indirect format. Detection of Polypeptide Markers or complexes thereof using antibodies can be done utilizing immunoassays which are run in either the forward, reverse or simultaneous modes. Examples of immunoassays are radioimmunoassays (RIA), enzyme immunoassays (e.g. ELISA), immunofluorescence, immunoprecipitation, latex agglutination, hemagglutination, histochemical tests, and sandwich (immunometric) assays. These terms are well understood by those skilled in the art. A person skilled in the art will know, or can readily discern, other immunoassay formats without undue experimentation.

Thus, the present invention provides means for determining one or more Polypeptide Marker in a sample by measuring one or more Polypeptide Marker by immunoassay. According to an embodiment of the invention, an immunoassay for detecting Polypeptide Markers in a biological sample comprises contacting antibodies that specifically bind to the Polypeptide Markers or complexes thereof in the sample under conditions that allow the formation of first complexes comprising antibodies and Polypeptide Markers or complexes and determining the presence or amount of the first complexes as a measure of the amount of Polypeptide Markers or complexes contained in the sample

It will be evident to a skilled artisan that a variety of immunoassay methods can be used to measure one or more Polypeptide Markers. In general, an immunoassay method may be competitive or noncompetitive.

In an aspect of the invention a competitive method is provided employing an immobilized or immobilizable antibody to a Polypeptide Marker and a labeled form of a Polypeptide Marker. Sample Polypeptide Marker and labeled Polypeptide Marker compete for binding to antibodies to the Polypeptide Marker. After separation of the resulting labeled Polypeptide Marker that has become bound to antibodies (bound fraction) from that which has remained unbound (unbound fraction), the amount of the label in either bound or unbound fraction is measured and may be correlated with the amount of a Polypeptide Marker in the test sample in any conventional manner, e.g., by comparison to a standard curve.

In another aspect, a non-competitive method is used for the determination of a Polypeptide Marker with the most common method being the “sandwich” method. In this assay, two antibodies to a Polypeptide Marker are employed. One of the antibodies to a Polypeptide Marker is directly or indirectly labeled (sometimes referred to as the “detection antibody”) and the other is immobilized or immobilizable (sometimes referred to as the “capture antibody”). The capture and detection antibodies can be contacted simultaneously or sequentially with the test sample. Sequential methods can be accomplished by incubating the capture antibody with the sample, and adding the detection antibody at a predetermined time thereafter (sometimes referred to as the “forward” method); or the detection antibody can be incubated with the sample first and then the capture antibody added (sometimes referred to as the “reverse” method). After the necessary incubation(s) have occurred, to complete the assay, the capture antibody is separated from the liquid test mixture, and the label is measured in at least a portion of the separated capture antibody phase or the remainder of the liquid test mixture. Generally it is measured in the capture antibody phase since it comprises Polypeptide Markers bound by (“sandwiched” between) the capture and detection antibodies. In an embodiment, the label may be measured without separating the capture antibodies and liquid test mixture.

The above-described immunoassay methods and formats are intended to be exemplary and are not limiting. Other methods now or hereafter developed for the determination of a Polypeptide Markers or complexes thereof are included within the scope hereof.

Binding agents (e.g. antibodies) may be used to detect and quantify Polypeptide Markers or complexes in a sample in order to diagnose and treat pathological states. In particular, antibodies may be used in immunohistochemical analyses, for example, at the cellular and sub-subcellular level, to detect Polypeptide Markers, to localize them to particular cells and tissues and to specific subcellular locations, and to quantitate the level of expression.

Antibodies specific for one or more Polypeptide Markers or complexes may be labelled with a detectable substance as described herein and localised in tissues and cells based upon the presence of the detectable substance. Cytochemical techniques known in the art for localizing antigens using light and electron microscopy may be used to detect Polypeptide Markers or complexes thereof.

Immunohistochemical methods for the detection of antigens in tissue samples are well known in the art. For example, immunohistochemical methods are described in Taylor, Arch. Pathol. Lab. Med. 102:112 (1978). Briefly, in the context of the present invention, a tissue sample obtained from a subject suspected of having a condition described herein is contacted with antibodies, preferably monoclonal antibodies recognizing Polypeptide Markers. The site at which the antibodies are bound is determined by selective staining of the sample by standard immunohistochemical procedures. The tissue sample may be normal tissue or abnormal/disease tissue.

### Diagnostic Methods for Conditions Involving Trophoblast Invasion

The invention in particular contemplates diagnostic methods for conditions associated with trophoblast cell death, differentiation, invasion, and/or cell fusion and turnover.

In an aspect, the invention provides methods for determining the presence or absence of a condition associated with trophoblast cell death, differentiation, invasion, and/or cell fusion and turnover, in a subject comprising (a) contacting a sample obtained from the subject with oligonucleotides that hybridize to Polynucleotide Markers, and (b) detecting in the sample levels of polynucleotides that hybridize to the Polynucleotide Markers relative to a predetermined cut-off value or standard, and therefrom determining the presence or absence of the condition in the subject. In a particular aspect, the Polynucleotide Markers comprise or are selected from the group consisting of polynucleotides encoding SMAD2, SMAD-3, SMAD7, ceruloplasmin, sFlt, Mtd-L, Mtd-P, Mcl-1c, Mcl-1L, TGFβ3, HIF1α, endoglin, PHD1, PHD2, PHD3, VHL, Siah1/2, and VEGF.

The present invention also provides a method for diagnosing in a subject a condition associated with trophoblast cell death, differentiation, invasion, and/or cell fusion and turnover and/or requiring regulation of trophoblast invasion, comprising detecting one or more Polypeptide Markers in a sample from the subject. In an aspect, the Polypeptide Markers comprise or are selected from the group consisting of SMAD2, phospho-SMAD2, SMAD-3, phospho-SMAD3, SMAD7, sFlt, ceruloplasmin, Mtd-L, Mtd-P, Mcl-1c, Mcl-1L, TGFβ3, endoglin, HIF1α, PHD1, PHD2, PHD3, VHL, Siah1/2, and VEGF.

In an embodiment of the diagnostic method of the invention, a method is provided for diagnosing increased risk of preeclampsia in a subject comprising detecting SMAD2, phospho-SMAD2, SMAD-3, phospho-SMAD3, SMAD7, sFlt, Mtd-L, Mtd-P, Mcl-1c, Mcl-1L, TGFβ3, HIF1α, endoglin, PHD1, PHD2, PHD3, VHL, Siah1/2, and VEGF in a sample from the subject.

In an aspect the markers can be contained on a nucleic acid or protein micro-array.

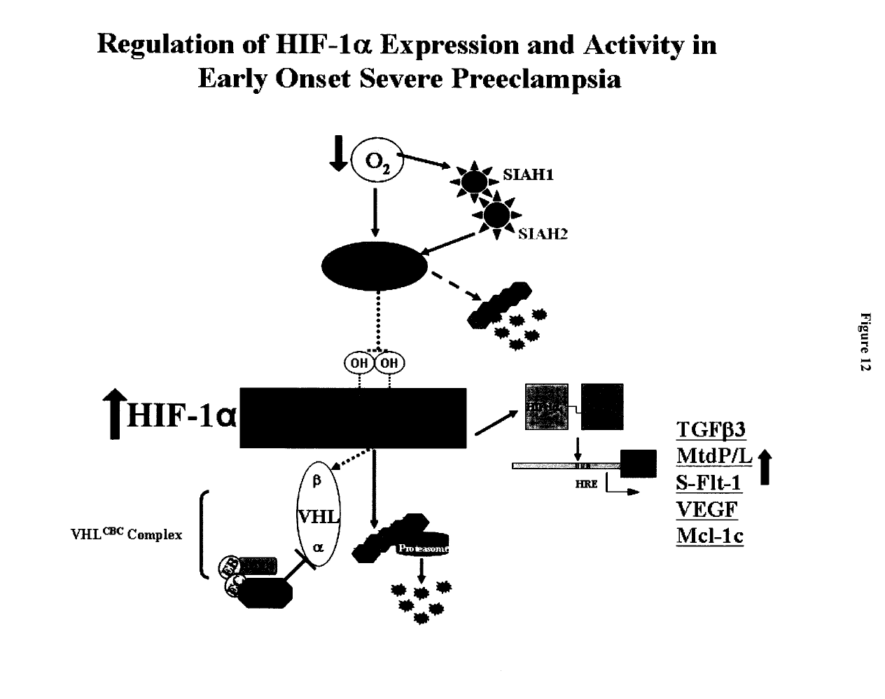

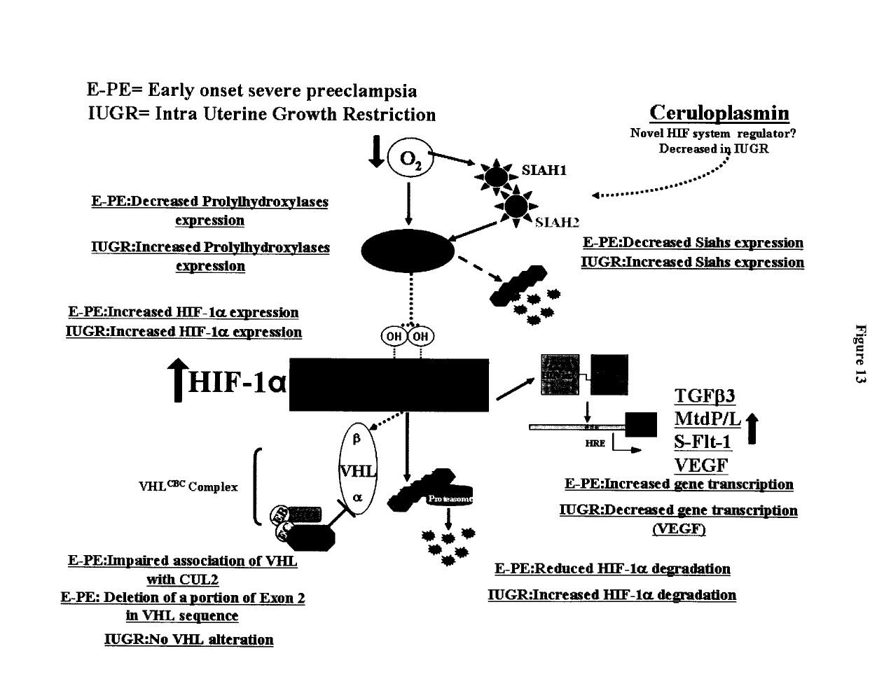

The diagnostic methods disclosed herein can be used to determine the presence or absence of preeclampsia or to determine the likelihood of occurrence of preeclampsia in a subject, or to distinguish subpathologies, including early onset severe preeclampsia (EPE) from late onset preeclampsia, or intrauterine growth restriction (IUGR). FIGS. 12 and 13 and Table 2 show the regulation of expression and activity of certain biomarkers in early onset severe preeclampsia and IUGR.

Pregnancies complicated by severe preeclampsia (ePE) are generally characterized by hypertension (systolic blood pressure≧140 mmHg; diastolic blood pressure≧90 mmHg), proteinuria (≧300 mg/24 h) and preterm delivery according to ACOG guidelines. Pregnancies complicated by severe Intrauterine Growth Restriction (sIUGR), disease are generally characterized by fetal growth<5° centile according to gestational age and sex, pathological umbilical Doppler flow velocimetry waveform (Absent End Diastolic Flow), and pathological bilateral uterine Doppler.

In an aspect, the invention contemplates a method for determining the likelihood of occurrence of preeclampsia in a pregnant mammal comprising detecting Polypeptide Markers or Polynucleotide Markers in a sample from the subject.

In an embodiment of the invention, a method is provided for diagnosing increased risk of preeclampsia in a subject comprising detecting Polypeptide Markers comprising or selected from the group consisting of SMAD2, phospho-SMAD2, SMAD-3, phospho-SMAD3, SMAD7, ceruloplasmin, sFlt, Mtd-L, Mtd-P, Mcl-1c, Mcl-1L, TGFβ3, HIF1α, endoglin, PHD1, PHD2, PHD3, VHL, Siah1/2, and/or VEGF, and/or polynucleotides encoding same, in a sample, and in particular using antibodies specific for the Polypeptide Markers. The Polypeptide Markers and/or polynucleotides encoding same, may be measured during the first trimester of pregnancy (approximately 1 to 15 weeks, 1 to 14 weeks, 1 to 12 weeks, 5 to 12 weeks, 5 to 8 weeks, 9 to 12 weeks, or 10 to 15 weeks). At least two measurements may be taken during this period, and subsequently including a measurement at about 12 to 16 or 14 to 16 weeks. If the levels are significantly different as compared to levels typical for women who do not suffer from preeclampsia, the patient is diagnosed as having an increased risk of suffering preeclampsia. Levels significantly different from (e.g. above) those typical for women who do not suffer from preeclampsia may be suspect and further monitoring and measurement of Polypeptide Markers may be appropriate. The information from the diagnostic method may be used to identify subjects who may benefit from a course of treatment, such as treatment via administration of inhibitors of one or more Polypeptide Markers.

The invention provides a method for diagnosing or identifying early onset severe preeclampsia, late onset preeclampsia, and intra-uterine growth restriction (IUGR) in a subject comprising detecting the Polypeptide Markers identified in Table 2, FIG. 12 or FIG. 13 and/or polynucleotides encoding same and diagnosing or identifying early onset severe preeclampsia, late onset preeclampsia, and intra-uterine growth restriction (IUGR) based on increases or decreases of the markers as indicated in Table 2, FIG. 12 or FIG. 13.

In a method for diagnosing or identifying early onset severe preeclampsia, higher levels of the markers, in particular significantly higher levels of one or more Polynucleotide Markers or Polypeptide Markers in patients compared to a control (e.g. normal) are indicative of early onset severe preeclampsia, or the likelihood of occurrence of early onset severe preeclampsia. In an embodiment, the invention relates to a method for diagnosing early onset severe preeclampsia in a subject comprising detecting SMAD2, phospho-SMAD2, SMAD-3, phospho-SMAD3, SMAD7, sFlt, ceruloplasmin, Mtd-L, Mtd-P, Mcl-1c, TGFβ3, HIF1α, endoglin, and/or VEGF and/or polynucleotides encoding same, in a sample from the subject.

The diagnostic methods can comprise diagnosing early onset preeclampsia using a panel of markers comprising or selected from the group consisting of Polynucleotide Markers or Polypeptide Markers, in particular SMAD2, phospho-SMAD2, SMAD-3, phospho-SMAD3, SMAD7, ceruloplasmin, sFlt, Mtd (in particular Mtd-P and Mtd-L), and Mcl-1 isoforms (in particular Mcl-1S or Mcl-1L, or caspase cleaved Mcl-1S or Mcl-1L, in particular caspase cleaved Mcl-1L, i.e., Mcl-1c), TGFβ3, TGFβ1, HIF1α, HIF1β, HIF2α, endoglin, PHD1, PHD2, PHD3, VHL, Siah1/2, syncytin, cullin 2, cleaved caspase (e.g. caspase-3), VEGF, FIH, NEDD8, Fas, and/or p53, or polynucleotides encoding the polypeptides.

In another method for diagnosing or identifying early onset severe preeclampsia, higher levels of HIF1α, SMAD2, phospho-SMAD2, SMAD-3, phospho-SMAD3, SMAD7, ceruloplasmin, sFlt, Mtd-L, Mtd-P, Mcl-1c, TGFβ3, endoglin and/or VEGF in patients compared to a control (e.g. normal) are indicative of early onset severe preeclampsia, or the likelihood of occurrence of early onset severe preeclampsia. In an aspect, the invention relates to a method for diagnosing early onset preeclampsia in a subject comprising detecting HIF1α, SMAD2, phospho-SMAD2, SMAD-3, phospho-SMAD3, SMAD7, ceruloplasmin, sFlt, Mtd-L, Mtd-P, Mcl-1c, TGFβ3, endoglin and/or VEGF in a sample from the subject.

The diagnostic methods can comprise diagnosing early onset preeclampsia using a panel of markers comprising or selected from the group consisting of HIF1α, SMAD2, phospho-SMAD2, SMAD-3, phospho-SMAD3, SMAD7, ceruloplasmin, sFlt, Mtd-L, Mtd-P, Mcl-1c, TGFβ3, endoglin, PHD1, PHD2, VHL, Siah1, and Siah2, cullin 2 and/or VEGF.

In an aspect of a diagnostic method for preeclampsia, one or more of the levels of Mtd-P, Mtd-L, and Mcl-1c and truncations thereof, SMAD2, SMAD-3, phospho-SMAD3, SMAD7, TGFβ3, NEDD8, ceruloplasmin, sFlt, endoglin and cullin2 are increased, and one or more of the levels of Mcl-1L, PHD1, PHD2, VHL, Siah1, and Siah2 are decreased compared to a control.

The invention provides a method for diagnosing early onset severe peeclampsia comprising comparing levels of at least two, three, four, five, six, seven, eight, nine or ten of SMAD2, phospho-SMAD2, SMAD-3, phospho-SMAD3, SMAD7, ceruloplasmin, sFlt, Mtd-P, Mtd-L, Mcl-1S, Mcl-1L, Mcl-1L truncation, TGFβ3, HIF1α, endoglin, PHD1, PHD2, NEDD8, cullin 2, cleaved caspase (e.g. caspase-3), Siah1/2, and VHL, and/or polynucleotides encoding same in a sample from a subject to the corresponding levels in a control. In a particular embodiment, the invention provides a method for diagnosing early onset peeclampsia comprising comparing levels of at least two, three, four, five, six, seven, eight, nine, or ten of SMAD2, phospho-SMAD2, SMAD-3, phospho-SMAD3, SMAD7, ceruloplasmin, sFlt, Mtd-P, Mtd-L, Mcl-1c, Mcl-1L, Mcl-1L truncation, TGFβ3, HIF1α, endoglin, PHD1, PHD2, NEDD8, cullin 2, cleaved caspase (e.g. caspase-3), Siah1/2, and VHL, and/or polynucleotides encoding same in a sample taken from a subject in the first trimester of pregnancy, in particular before 14, 12, 10, 8, or 5 weeks, to the corresponding levels in a control. In a particular embodiment, the invention provides a method for diagnosing early onset severe peeclampsia comprising comparing levels of SMAD2, SMAD7, sFlt, Mtd-P, Mtd-L, Mcl-1c, TGFβ3, HIF1α, endoglin, and VEGF and/or polynucleotides encoding same in a sample taken from a subject in the first trimester of pregnancy, in particular before 14, 12, 10, 8, or 5 weeks, to the corresponding levels in a control. The control may be a pre-term or normotensive age-matched control or a sample taken at different stage of pregnancy.

In a particular embodiment, a significant increase in Mtd-P, Mtd-L, sFlt, SMAD2, SMAD-3, phospho-SMAD3, SMAD7, VEGF, Mcl-1c, TGFβ3, ceruloplasmin, and HIF1α and/or polynucleotides encoding same is indicative of early onset preeclampsia. In another particular embodiment, a significant increase in Mtd-P, Mtd-L, sFlt, SMAD2/7, VEGF, Mcl-1c, TGFβ3, endoglin and HIF1α and/or polynucleotides encoding same, and/or a significant decrease in PHD1, PHD2, Siah1/2, Mcl-11, NEDD8, cullin 2, and/or VHL and/or polynucleotides encoding same, is indicative of early onset preeclampsia. In a further particular embodiment, a 2 to 10 fold, 2 to 8 fold, 2 to 5 fold, 2 to 4 fold, or 3 to 3.5 fold increase in Mtd-P, Mtd-L, sFlt, SMAD2, SMAD3, SMAD7, ceruloplasmin, VEGF, Mcl-1c, TGFβ3 and HIF1α and/or polynucleotides encoding same compared to a control is indicative of early onset preeclampsia.

The invention also provides a method for diagnosing late onset preeclampsia comprising comparing levels of in Mtd-P, Mtd-L, ceruloplasmin, sFlt, SMAD2, SMAD3, SMAD7, VEGF, Mcl-1c, TGFβ3, VHL, Siah1/2, PHD1, PHD2, PHD3, and HIF1α and/or polynucleotides encoding same in a sample from a subject to the corresponding levels in a control. In an aspect, the invention provides a method for diagnosing late onset preeclampsia comprising comparing levels of in Mtd-P, Mtd-L, sFlt, SMAD2, SMAD7, Mcl-1c, Mcl-1L, TGFβ3, VEGF, VHL, Siah1/2, PHD1, PHD2, PHD3, and HIF1α and/or polynucleotides encoding same in a sample from a subject to the corresponding levels in a control. The sample is generally taken from a subject in the third trimester of pregnancy, in particular after week 20 or 25.

The invention also provides a method for diagnosing intra-uterine growth restriction (IUGR) comprising comparing levels of Polypeptide Markers and Polynucleotide Markers, in particular, Mtd-L, Mtd-P, sFLt, SMAD2, phospho-SMAD2, SMAD-3, phospho-SMAD3, SMAD7, VEGF, Mcl-1L, Mcl-1c, HIF1α, VHL, TGFβ3, PHD1, PHD2, PHD3, endoglin and Siah1/2, and/or polynucleotides encoding same, in a sample from a subject to the corresponding levels in a control. In an embodiment, the Markers are sFLt, SMAD2, SMAD7, VEGF, HIF1α, VHL, TGFβ3, PHD1, PHD2, PHD3, and Siah1/2, and/or polynucleotides encoding same. The sample is generally taken from a subject in the third trimester of pregnancy, in particular after about week 20 or 25. An increase in HIF1α, TGFβ3, phospho-SMAD2, phospho-SMAD3, sFlt, endoglin, PHD1, PHD2, PHD3, and/or Siah1/2 and/or polynucleotides encoding same, and optionally a decrease in VEGF, SMAD7, and/or ceruloplasmin can be indicative of IUGR.

The invention further provides a method for diagnosing severe IUGR, comprising comparing levels of PHD1, PHD2, and/or PHD3, Siah1, Siah2, SMAD2, SMAD3, HIF1α, TGFβ3, endoglin, and sFlt, and/or polynucleotides encoding same, in a sample from a subject to the corresponding levels in a control. In an aspect the samples are taken from a subject at or later than about 20, 25, 30, or 35 weeks.

The invention further provides a method for diagnosing severe IUGR, comprising comparing levels of PHD1, PHD2, and/or PHD3, and optionally Siah1, Siah2, VEGF, FIH, and/or HIF1α and/or polynucleotides encoding same, in a sample from a subject to the corresponding levels in a control. An increase, in particular a significant increase in levels of one or both of PHD1, PHD2, PHD3, Siah1, Siah2, sFlt, TGFβ3, HIFα, and FIH, and/or polynucleotides encoding same, and/or decreased levels of VEGF or a polynucleotide encoding same can be indicative of IUGR. In an aspect the samples are taken from a subject at or later than about 20, 25, 30, or 35 weeks.

The invention further provides a method for diagnosing severe IUGR, comprising comparing levels of phospho-SMAD2, phospho-SMAD3, SMAD7 and optionally PHD1, PHD2, PHD3, Siah1, Siah2, VEGF, FIH, endoglin, and/or HIF1α and/or polynucleotides encoding same, in a sample from a subject to the corresponding levels in a control. An increase, in particular a significant increase in levels of one or both of phospho-SMAD2 and phospho-SMAD3, and optionally PHD1, PHD2, PHD3, Siah1, Siah2, sFlt, TGFβ3, HIFα, endoglin and FIH, and/or polynucleotides encoding same, and/or decreased levels of SMAD7 and/or VEGF or a polynucleotide encoding same can be indicative of IUGR. In an aspect the samples are taken from a subject at or later than about 20, 25, 30, or 35 weeks.

The invention also contemplates a method for monitoring the progression of preeclampsia or subpathologies thereof in an individual, comprising:
(a) contacting an amount of binding agents (e.g., an antibody) which bind to Polypeptide Markers with a sample from the individual so as to form a binary complex comprising the binding agent and Polypeptide Markers in the sample;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of the progression of the preeclampsia in said individual.

The amount of complexes may also be compared to a value representative of the amount of the complex. In an embodiment an increase in complexes in (c) is indicative of preeclampsia.

It will also be appreciated that the diagnostic methods disclosed herein may also be useful in the diagnosis or monitoring of choriocarcinoma or hydatiform mole which involves uncontrolled trophoblast invasion. Further the above methods may be used to diagnose or monitor other pregnancy complications including molar pregnancy, preterm labour, preterm birth, fetal anomalies, and placental abruption.

In an aspect, the invention provides a method for diagnosing molar pregnancies comprising comparing levels of Markers in a sample from a subject to the corresponding levels in a control.

Polypeptides and polynucleotides may be detected in patient samples using the methods disclosed herein. In particular, a polypeptide to be analyzed in a method of the invention may be detected using a binding agent or a substance which directly or indirectly interacts with the polypeptide. For example, antibodies specific for Polypeptide Markers may be used to diagnose and monitor a condition associated with abherrant trophoblast cell death, differentiation, invasion, and/or cell fusion and turnover or requiring regulation of trophoblast invasion. A method of the invention using antibodies may utilize Countercurrent immuno-Electrophoresis (CIEP), Radioimmunoassays, Radioimmunoprecipitations, and Enzyme-Linked Immuno-Sorbent Assays (ELISA), Dot Blot assays, Inhibition or Competition assays and sandwich assays as described herein and known in the art.

The presence of Polypeptide Markers in a sample may also be determined by measuring the binding of the Polypeptide Markers with substances that are known to bind to same. A binding agent may be labeled using conventional methods with various detectable substances such as enzymes, fluorescent materials, luminescent materials and radioactive materials which are described herein and known to a person skilled in the art. A binding agent may also be indirectly labeled with a ligand binding partner. For example, the antibodies, or a binding agent may be conjugated to one partner of a ligand binding pair, and the polypeptide may be coupled to the other partner of the ligand binding pair. Representative examples of binding partners are described herein.

The antibodies or binding agents used in the method of the invention may be insolubilized as described herein. Indirect methods may also be employed in which a primary antigen-antibody reaction is amplified by the introduction of a second antibody, having specificity for the antibody reactive against the cytokine.

A Polynucleotide Marker can be detected in a diagnostic method of the invention using nucleotide probes. Suitable probes include polynucleotides based on nucleic acid sequences encoding the Polypeptide Markers. A nucleotide probe may be labeled with a detectable substance as described herein.

A Polynucleotide Marker can also be detected by selective amplification of the polynucleotide using polymerase chain reaction (PCR) methods. Synthetic oligonucleotide primers can be constructed from the sequences of Polynucleotide Markers using conventional methods. A nucleic acid can be amplified in a sample using these oligonucleotide primers and standard PCR amplification techniques.

### Kits

The invention also relates to kits for carrying out the methods of the invention. In an aspect, the invention provides a test kit for diagnosing a condition requiring regulation of trophoblast cell death, differentiation, invasion, and/or cell fusion and turnover, in particular preeclampsia, IUGR, choriocarcinoma, hydatiform mole, or a molar pregnancy, which comprises a binding agent that interacts with Polynucleotide Markers, or a polynucleotide that interacts with Polynucleotide Markers.

A kit can comprise instructions, negative and positive controls, and means for direct or indirect measurement of Markers. Kits may typically comprise two or more components required for performing a diagnostic assay. Components include but are not limited to compounds, reagents, containers, and/or equipment.

The methods described herein may be performed by utilizing pre-packaged diagnostic kits comprising one or more specific Polypeptide Marker disclosed herein or binding agent (e.g. antibody) described herein, which may be conveniently used, e.g., in clinical settings to screen and diagnose patients and to screen and identify those individuals exhibiting a predisposition to a condition disclosed herein, in particular preeclampsia.

In an embodiment, a container with a kit comprises one or more binding agent as described herein. By way of example, the kit may contain antibodies or antibody fragments which bind specifically to epitopes of Polypeptide Markers; antibodies against the antibodies labelled with an enzyme; and, a substrate for the enzyme. The kit may also contain microtiter plate wells, standards, assay diluent, wash buffer, adhesive plate covers, and/or instructions for carrying out a method of the invention using the kit.

In an aspect of the invention, the kit includes antibodies or fragments of antibodies which bind specifically to an epitope of one or more Polypeptide Marker, and means for detecting binding of the antibodies to their epitope associated with a condition disclosed herein, either as concentrates (including lyophilized compositions), which may be further diluted prior to use or at the concentration of use, where the vials may include one or more dosages.

A kit may be designed to detect the level of polynucleotides encoding one or more Polynucleotide Markers disclosed herein in a sample. Such kits generally comprise oligonucleotide probes or primers, as described herein, that hybridize to Polynucleotide Markers. Such an oligonucleotide may be used, for example, within a PCR or hybridization procedure.

The invention provides a kit containing a micoarray described herein ready for hybridization to target Polynucleotide Markers plus software for the data analysis of the results. The software to be included with the kit comprises data analysis methods, in particular mathematical routines for marker discovery, including the calculation of correlation coefficients between clinical categories and marker expression. The software may also include mathematical routines for calculating the correlation between sample marker expression and control marker expression, using array-generated fluorescence data, to determine the clinical classification of the sample.

In an aspect, the invention provides a kit comprising a reagent that detects Polypeptide Markers or Polynucleotide Markers, and instructions or package insert or label for assaying whether a pregnant mammal is at risk of early onset preeclampsia. The kit may further comprise a detection means and/or microtiter plates, standard or tracer, and an immobilized reagent that detects Polypeptide Markers or Polynucleotide Markers and is used to capture the Polypeptide Markers or Polynucleotide Markers.

The invention contemplates a kit for assessing the presence of cells and tissues associated with a condition disclosed herein, wherein the kit comprises antibodies specific for one or more Polypeptide Markers or complexes thereof, or primers or probes for Polynucleotide Markers, and optionally probes, primers or antibodies specific for other markers associated with the condition.

The reagents suitable for applying the screening methods of the invention to evaluate compounds may be packaged into convenient kits described herein providing the necessary materials packaged into suitable containers.

The invention relates to a kit for assessing the suitability of each of a plurality of test compounds for inhibiting a condition disclosed herein. In an aspect, the kit comprises reagents for assessing one or more Polypeptide Markers or Polynucleotide Markers, and optionally a plurality of test agents or compounds.

Additionally the invention provides a kit for assessing the potential of a test compound to contribute to a condition disclosed herein. In an aspect, the kit comprises cells and tissues associated with the condition and reagents for assessing one or more Polypeptide Markers or Polynucleotide Markers.

### Computer Systems

Analytic methods contemplated herein can be implemented by use of computer systems and methods described below and known in the art. Thus, the invention provides computer readable media comprising one or more Polypeptide Markers or Polynucleotide Markers. “Computer readable media” refers to any medium that can be read and accessed directly by a computer, including but not limited to magnetic storage media, such as floppy discs, hard disc storage medium, and magnetic tape; optical storage media such as CD-ROM; electrical storage media such as RAM and ROM; and hybrids of these categories such as magnetic/optical storage media. Thus, the invention contemplates computer readable medium having recorded thereon markers identified for patients and controls.

“Recorded” refers to a process for storing information on computer readable medium. The skilled artisan can readily adopt any of the presently known methods for recording information on computer readable medium to generate manufactures comprising information on one or more markers disclosed herein.

A variety of data processor programs and formats can be used to store information on one or more markers, and/or polynucleotides encoding one or more markers. For example, the information can be represented in a word processing text file, formatted in commercially-available software such as WordPerfect and MicroSoft Word, or represented in the form of an ASCII file, stored in a database application, such as DB2, Sybase, Oracle, or the like. Any number of dataprocessor structuring formats (e.g., text file or database) may be adapted in order to obtain computer readable medium having recorded thereon the marker information.

By providing the marker information in computer readable form, one can routinely access the information for a variety of purposes. For example, one skilled in the art can use the information in computer readable form to compare marker information obtained during or following therapy with the information stored within the data storage means.

The invention provides a medium for holding instructions for performing a method for determining whether a patient has a condition disclosed herein or a pre-disposition to a condition disclosed herein, comprising determining the presence or absence of one or more markers, and based on the presence or absence of the Markers, determining the condition or a pre-disposition to a condition, optionally recommending a procedure or treatment.

The invention also provides in an electronic system and/or in a network, a method for determining whether a subject has a condition disclosed herein, or a pre-disposition to a condition disclosed herein, comprising determining the presence or absence of one or more Markers, and based on the presence or absence of the Markers, determining whether the subject has the condition or a pre-disposition to the condition, and optionally recommending a procedure or treatment.

The invention further provides in a network, a method for determining whether a subject has a condition disclosed herein or a pre-disposition to a condition disclosed herein comprising: (a) receiving phenotypic information on the subject and information on one or more Markers associated with samples from the subject; (b) acquiring information from the network corresponding to the Markers; and (c) based on the phenotypic information and information on the Markers, determining whether the subject has the condition or a pre-disposition to the condition, and (d) optionally recommending a procedure or treatment.

The invention still further provides a system for identifying selected records that identify a diseased cell or tissue. A system of the invention generally comprises a digital computer; a database server coupled to the computer; a database coupled to the database server having data stored therein, the data comprising records of data comprising one or more Markers, and a code mechanism for applying queries based upon a desired selection criteria to the data file in the database to produce reports of records which match the desired selection criteria.

The invention contemplates a business method for determining whether a subject has a condition disclosed herein or a pre-disposition to a condition disclosed herein comprising: (a) receiving phenotypic information on the subject and information on one or more Markers associated with samples from the subject; (b) acquiring information from a network corresponding to the Markers; and (c) based on the phenotypic information, information on the Markers and acquired information, determining whether the subject has the condition or a pre-disposition to the condition, and optionally recommending a procedure or treatment.

In an aspect of the invention, the computer systems, components, and methods described herein are used to monitor a condition or determine the stage of a condition.

### Therapeutic Applications

The invention contemplates therapeutic applications associated with the Markers disclosed herein including conditions, diseases or disorders requiring modulation of, or involving, trophoblast cell death, differentiation, invasion, and/or cell fusion and turnover (also referred to herein as “condition(s)”. Examples of such conditions are preeclampsia including early onset severe preeclampsia (EPE) and late onset preeclampsia (LPE), intra-uterine growth restriction (IUGR), choriocarcinoma, hydatiform mole, and molar pregnancy.

One or more Polypeptide Markers and/or Polynucleotide Markers, in particular SMAD2, phospho-SMAD2, phospho-SMAD2, phospho-SMAD3, SMAD7, and/or ceruloplasmin, may be targets for immunotherapy. Immunotherapeutic methods include the use of antibody therapy. In one aspect, the invention provides one or more antibodies that may be used to prevent a condition requiring modulation of or involving trophoblast cell death, differentiation, invasion, and/or cell fusion and turnover (e.g. preeclampsia) associated with the marker. In another aspect, the invention provides a method of preventing, inhibiting or reducing a condition requiring modulation of or involving trophoblast cell death, differentiation, invasion, and/or cell fusion and turnover (e.g. preeclampsia) comprising administering to a patient an antibody which binds specifically to one or more Polypeptide Markers and/or Polynucleotide Markers, in particular SMAD2, phospho-SMAD2, phospho-SMAD2, phospho-SMAD3, SMAD7, and/or ceruloplasmin, in an amount effective to prevent, inhibit, or reduce the condition or the onset of the condition.

The methods of the invention contemplate the administration of single antibodies as well as combinations, or “cocktails”, of different individual antibodies such as those recognizing different epitopes of other markers. Such cocktails may have certain advantages inasmuch as they contain antibodies that bind to different epitopes of Polypeptide Markers and/or exploit different effector mechanisms. Such antibodies in combination may exhibit synergistic therapeutic effects. In addition, the administration of one or more marker specific antibodies may be combined with other therapeutic agents. The specific antibodies may be administered in their “naked” or unconjugated form, or may have therapeutic agents conjugated to them.

The marker specific antibodies used in the methods of the invention may be formulated into pharmaceutical compositions comprising a carrier suitable for the desired delivery method. Suitable carriers include any material which when combined with the antibodies retains the function of the antibody and is non-reactive with the subject's immune systems. Examples include any of a number of standard pharmaceutical carriers such as sterile phosphate buffered saline solutions, bacteriostatic water, and the like (see, generally, Remington's Pharmaceutical Sciences 16th Edition, A. Osal., Ed., 1980).

One or more marker specific antibody formulations may be administered via any route capable of delivering the antibodies to the site or injury. Routes of administration include, but are not limited to, intravenous, intraperitoneal, intramuscular, intradermal, and the like. Antibody preparations may be lyophilized and stored as a sterile powder, preferably under vacuum, and then reconstituted in bacteriostatic water containing, for example, benzyl alcohol preservative, or in sterile water prior to injection.

Treatment will generally involve the repeated administration of the antibody preparation via an acceptable route of administration at an effective dose. Dosages will depend upon various factors generally appreciated by those of skill in the art, including the etiology of the condition, stage of the condition, the binding affinity and half life of the antibodies used, the degree of marker expression in the patient, the desired steady-state antibody concentration level, frequency of treatment, and the influence of any therapeutic agents used in combination with a treatment method of the invention. A determining factor in defining the appropriate dose is the amount of a particular antibody necessary to be therapeutically effective in a particular context. Repeated administrations may be required to achieve a desired effect. Direct administration of one or more marker antibodies is also possible and may have advantages in certain situations.

Patients may be evaluated for Markers in order to assist in the determination of the most effective dosing regimen and related factors. The assay methods described herein, or similar assays, may be used for quantitating marker levels in patients prior to treatment. Such assays may also be used for monitoring throughout therapy, and may be useful to gauge therapeutic success in combination with evaluating other parameters such as levels of Markers.

A Polynucleotide Marker, in particular a polynucleotide encoding SMAD2, phospho-SMAD2, phospho-SMAD2, phospho-SMAD3, SMAD7, and/or ceruloplasmin, associated with a condition disclosed herein can be turned off by transfecting a cell or tissue with vectors that express high levels of the Polynucleotide Marker. Such constructs can inundate cells with untranslatable sense or antisense sequences. Even in the absence of integration into the DNA, such vectors may continue to transcribe RNA molecules until all copies are disabled by endogenous nucleases. Vectors derived from retroviruses, adenovirus, herpes or vaccinia viruses, or from various bacterial plasmids, may be used to deliver Polynucleotide Markers to a targeted organ, tissue, or cell population. Methods well known to those skilled in the art may be used to construct recombinant vectors that will express Polynucleotide Markers such as antisense. (See, for example, the techniques described in Sambrook et al (supra) and Ausubel et al (supra).)

Methods for introducing vectors into cells or tissues include those methods discussed herein and which are suitable for in vivo, in vitro and ex vivo therapy. For example, delivery by transfection or by liposome are well known in the art.

Modifications of gene expression can be obtained by designing antisense molecules, DNA, RNA or PNA, to the regulatory regions of Polynucleotide Markers, i.e., the promoters, enhancers, and introns. Preferably, oligonucleotides are derived from the transcription initiation site, e.g. between −10 and +10 regions of the leader sequence. The antisense molecules may also be designed so that they block translation of mRNA by preventing the transcript from binding to ribosomes. Inhibition may also be achieved using “triple helix” base-pairing methodology. Triple helix pairing compromises the ability of the double helix to open sufficiently for the binding of polymerases, transcription factors, or regulatory molecules. Therapeutic advances using triplex DNA are reviewed by Gee J E et al (In: Huber B E and B I Carr (1994) Molecular and Immunologic Approaches, Futura Publishing Co, Mt Kisco N.Y.).

Ribozymes are enzymatic RNA molecules that catalyze the specific cleavage of RNA. Ribozymes act by sequence-specific hybridization of the ribozyme molecule to complementary target RNA, followed by endonucleolytic cleavage. The invention therefore contemplates engineered hammerhead motif ribozyme molecules that can specifically and efficiently catalyze endonucleolytic cleavage of Polynucleotide Markers, in particular polynucleotides encoding SMAD2, phospho-SMAD2, phospho-SMAD2, phospho-SMAD3, SMAD7, and/or ceruloplasmin.

Specific ribozyme cleavage sites within any potential RNA target may initially be identified by scanning the target molecule for ribozyme cleavage sites which include the following sequences, GUA, GUU and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

One or more Markers (e.g. down-regulated Markers), and fragments thereof, may be used to prevent, treat, or reduce a condition requiring modulation of or involving trophoblast cell death, differentiation, invasion, and/or cell fusion and turnover (e.g. preeclampsia) in a subject. The markers may be formulated into compositions for administration to subjects with a pre-disposition for or suffering from a condition requiring modulation of or involving trophoblast cell death, differentiation, invasion, and/or cell fusion and turnover (e.g. preeclampsia). Therefore, the present invention also relates to a composition comprising one or more Markers or a fragment thereof, in particular SMAD2, phospho-SMAD2, SMAD3, phospho-SMAD3, SMAD7, and/or ceruloplasmin, and a pharmaceutically acceptable carrier, excipient or diluent. A method for treating or preventing a condition requiring modulation of or involving trophoblast cell death, differentiation, invasion, and/or cell fusion and turnover (e.g. preeclampsia) in a subject is also provided comprising administering to a patient in need thereof, one or more Markers, in particular SMAD2, phospho-SMAD2, SMAD3, phospho-SMAD3, SMAD7, and/or ceruloplasmin.

The invention further provides a method of preventing, inhibiting, or reducing a condition requiring modulation of or involving trophoblast cell death, differentiation, invasion, and/or cell fusion and turnover (e.g. preeclampsia) in a patient comprising:
(a) obtaining a sample comprising tissue or cells associated with the condition from the patient;
(b) separately maintaining aliquots of the sample in the presence of a plurality of test agents;
(c) comparing levels of one or more Markers, in particular SMAD2, phospho-SMAD2, SMAD3, phospho-SMAD3, SMAD7, and/or ceruloplasmin, in each aliquot;
(d) administering to the patient at least one test agent which alters the levels of the Markers in the aliquot containing that test agent, relative to the other test agents.

An active therapeutic substance described herein may be administered in a convenient manner such as by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance. Solutions of an active compound as a free base or pharmaceutically acceptable salt can be prepared in an appropriate solvent with a suitable surfactant. Dispersions may be prepared in glycerol, liquid polyethylene glycols, and mixtures thereof, or in oils.

A composition described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington: The Science and Practice of Pharmacy (21^{st} Edition. 2005, University of the Sciences in Philadelphia (Editor), Mack Publishing Company), and in The United States Pharmacopeia: The National Formulary (USP 24 NF19) published in 1999. On this basis, the compositions include, albeit not exclusively, solutions of the active substances in association with one or more pharmaceutically acceptable vehicles or diluents, and contained in buffered solutions with a suitable pH and iso-osmotic with the physiological fluids.

A composition is indicated as a therapeutic agent either alone or in conjunction with other therapeutic agents or other forms of treatment. The compositions of the invention may be administered concurrently, separately, or sequentially with other therapeutic agents or therapies.

The therapeutic activity of compositions and agents/compounds identified using a method of the invention and may be evaluated in vivo using a suitable animal model.

The methods of the invention for use on subjects contemplate prophylactic as well as therapeutic or curative use. In embodiments of the invention, the methods and compositions described herein are used prophylactically to prevent development of a condition requiring modulation of or involving trophoblast cell death, differentiation, invasion, and/or cell fusion and turnover (e.g. preeclampsia or IUGR).

Typical subjects for treatment include persons susceptible to, suffering from or that have suffered from a condition requiring modulation of or involving trophoblast cell death, differentiation, invasion, and/or cell fusion and turnover (e.g. preeclampsia). Subjects or patients include warm-blooded animals such as mammals. In particular, the terms refer to a human. A subject, or patient may be afflicted with or suspected of having or being pre-disposed to a condition requiring modulation of or involving trophoblast cell death, differentiation, invasion, and/or cell fusion and turnover (e.g. preeclampsia). The present invention may be particularly useful for determining at-risk patients.

The following non-limiting examples are illustrative of the present invention:

### EXAMPLE 1

The aims of this study were to examine placental sFlt-1 expression in models of placental hypoxia including placental tissues obtained from various weeks of gestations across the first trimester (in vivo developmental hypoxia), from high altitude (in vivo physiological chronic hypoxia) and preeclamptic pregnancies (in vivo pathological hypoxia). Additionally, using a well-established villous organ culture system the mechanisms by which reduced oxygenation (in vitro hypoxia) and hypoxia-reoxygenation regulate sFlt-1 expression were investigated. Finally, the study was aimed at determining if HIF, in particular HIF-1α, plays a direct role in regulating the expression of sFlt-1 in the human placenta.

The following materials and methods were used in the study described in the example.

### Materials and Methods

### Tissue and Blood Collection

Local Ethics Committee approval was obtained for the study from the participating institutions and all women gave written informed consent. Tissue collection strictly adhered to the guidelines outlined in The Declaration of Helsinki. High altitude placentae and blood samples were collected in Leadville (HA, 3179 meters above sea level), Colorado, USA. HA placentae were obtained from healthy normotensive patients at term. Ten mL of blood was withdrawn from the mother's antecubital vein at 36 weeks or greater of gestation, and at 3 months post-partum. Blood was allowed to clot and the serum was separated and stored in −80° C. for later analysis. Sea level placental samples (SL, also referred to as term control; TC) were collected from term deliveries in Toronto, Ontario, Canada (˜40 m). Control blood samples were collected in Denver, Colo., USA (1600 m). Severe early-onset preeclampsia was diagnosed based on the American College of Obstetrics and Gynecology (ACOG) criteria (1). Preeclamptic placentae (PE, n=16) and preterm normotensive age-matched control placentae (PTC, n=12) were collected from deliveries at Mount Sinai Hospital in Toronto. Early-onset preeclampsia was defined when the patient was delivered before 34 weeks gestation due to preeclampsia. All third trimester specimens were obtained immediately after delivery from normal looking cotyledons randomly collected. Areas with calcified, necrotic or visually ischemic tissue were omitted from sampling. Subjects suffering from diabetes, essential hypertension or renal disease were excluded. Pregnancies affected by IUGR were also excluded. Preterm deliveries were due to multiple pregnancies (16%), preterm labor due to incompetent cervix (35%), premature preterm rupture of membrane (33%) and spontaneous preterm deliveries without identifiable cause (16%). All preterm and term control groups did not show clinical or pathological signs of preeclampsia, infections or other maternal or placental disease. Birth weight, gestational age and laboratory values or clinical observations relevant to the health of the mother were abstracted from the clinical records. The clinical characteristics of the patients are shown in Table 1. First trimester placental samples (5-12 weeks of gestation, n=33) were collected from elective first trimester pregnancy terminations performed by dilatation and curettage. Gestational age was determined by the date of the last menstrual period and first trimester ultrasound measurement of crown-rump-length (CRL).

### First Trimester Human Chorionic Villous Explant Culture

Chorionic villous explant culture was performed as previously described (Caniggia, 2000). Briefly, placental tissues (5-8 weeks of gestation, 15 separate sets) were placed in ice-cold PBS and processed within 2 hours of collection. Tissues were aseptically dissected to remove decidual tissue and fetal membranes. Small fragments of placental villi (25-45 mg wet weight) were teased apart, placed on Millicell-CM culture dish inserts (Millipore Corp., Bedford, Mass., USA) pre-coated with 0.15 mL of undiluted Matrigel (Collaborative Biomedical Products, Bedford, Mass., USA), and transferred to a 24-well culture dish. Explants were cultured in serum-free DMEM/F12 (Gibco BRL, Grand Island, N.Y., USA) supplemented with 100 μg/mL streptomycin, 100 U/mL penicillin, and incubated overnight at 37° C. in 5% CO₂ in air to allow attachment. Explants were maintained in standard condition (5% CO₂ in 95% air) or in an atmosphere of 3% or 8% O₂ (92% or 87% N₂ and 5% CO₂, respectively) for 72 hrs at 37° C. The morphological integrity and viability of villous explants and their EVT outgrowth and migration were monitored daily for up to 5 days as previously reported (Caniggia, 2000). Explants from more than 10 different placentae in more than 15 separate experiments were used. In parallel experiments, hypoxia/re-oxygenation (HR) was performed as previously described (15) by decreasing from 8% O₂ (physiological oxygen tension at 10-12 weeks' gestation) to 2-3% O₂ for 2 hours, followed by exposure to 20% O₂ standard conditions for 1 hour. At least three independent experiments using triplicate samples for each condition were performed.

### Pharmacological Stabilization and Knockdown of HIF-1

Explants kept in 20% O₂ were treated with a 1.0 mM concentration of dimethyloxalyl-glycin (DMOG), an inhibitor of prolyl-hydroxylases activity mimicking hypoxia via stabilization of HIF-1α (16). Control cultures maintained in standard 20% O₂ conditions were run in parallel in the presence of medium alone. HIF-1α knockdown studies were performed using a previously validated antisense approach (Caniggia, 2000). In brief, 10 μM of HIF-1α antisense and control sense oligos were added to the media of explants in 3% oxygen conditions. After completion of experiments, conditioned media were collected and kept in −80° C. for ELISA analysis of sFlt-1. Explants were collected and snap frozen in liquid nitrogen for gene analysis. Explants in each experiment were also fixed in 4% paraformaldehyde for immunohistochemical analysis.

### TUNEL Assay

An in situ cell death detection kit was purchased from Amersham Biosciences, NJ, USA. Terminal deoxynucleotidyl transferase-dUTP-nick end labelling (TUNEL) assays were performed according to instructions provided by the manufacturer. Paraffin sections of tissue were dewaxed in xylene, rehydrated in descending grades of ethanol, and finally soaked in PBS. Tissue sections were pre-treated with Proteinase K (10 μg/mL) in PBS for 10 min, washed and then incubated in 3% hydrogen peroxide in methanol for 45 minutes. After washing in PBS, the slides were preincubated with 1× One Phor All buffer in 0.1% Triton X-100 in water for 30 minutes and then incubated in TdT solution for 1.5 hour at 37° C. Samples were washed in PBS and avidin biotin complex (Vector Laboratories, Burlingame, Calif.) was applied for 1 hour. Staining was detected with the diamino-benzidine chromogen after 10 minutes. Slides were counterstained with haematoxylin, dehydrated in ascending concentrations of ethanol, and fixed with xylene.

RNA Isolation and Quantitation Using Real-Time RT-PCR (qPCR)

Total RNA was isolated from placental samples using a Trizol-based approach according to the manufacturer's protocol (Invitrogen, CA). Total RNA was isolated from whole explants using RNeasy Mini Kit (Qiagen, Ontario) according to the manufacturer's protocol. DNA contamination was enzymatically removed by DNase-I digestion before RNA reverse transcription. qPCR (sFlt-1) was performed on the MJ's Opticon II® Light Cycler system as previously described (36). TaqMan Universal MasterMix and specific Taqman®primers and probe for sVEGFR-1 (sFlt-1, accession number: U01134) and 18S were used (Applied Biosystems, CA) based on the manufacturer's protocol (Applied Biosystems, CA). Relative quantitation of data was performed using logarithmic curves. Expression level of sFlt-1 was normalized based on 18S expression using the 2ΔΔCt formula as previously described (25, 36). Sequences for sFlt-1 primers and probe: Forward: 5′-GGGAAGAAATCCTCCAGAAGAAAGA-3′ [SEQ ID NO. 1]; Reverse: 5′-GAGATCCGAGAGAAAACAGCCTTT-3′ [SEQ ID NO. 2]; Probe: 5′-CAGTGCTCACCTCTGATTG-3′ [SEQ ID NO. 3]. Total amplicon size is 79 base pares.

### Western Blot Analysis

Western blot analysis for sFlt-1 was performed using 50 μg of total placental protein lysates that were subjected to 6% (wt/vol) SDS-PAGE gel. After electrophoresis, proteins were transferred to PVDF membranes. Nonspecific binding was blocked by incubation in 5% (wt/vol) bovine serum albumin (BSA) in Tris-buffered saline containing 0.1% (vol/vol) Tween-20 (TBST) for 60 minutes. Membranes were then incubated with 1:200 diluted specific anti-soluble VEGFR-1 antibody (Zymed Laboratories, San Francisco, Calif.), in 5% (wt/vol) BSA in TBST at 4° C. After overnight incubation, membranes were washed with TBST and incubated for 60 minutes at room temperature with 1:10,000 diluted horseradish peroxidase-conjugated anti-rabbit IgG (Santa Cruz, Calif.) in 5% (wt/vol) BSA in TBST. After washing with TBST, blots were exposed to chemiluminescent reagent (ECL; Amersham Pharmacia Biotech, Oakville, Ontario, Canada). All western blots were checked for equal protein loading at all times using ponceau staining.

### Immunohistochemistry (IHC)

Paraffin sections were mounted on glass slides, dewaxed in xylene and rehydrated in descending ethanol gradient. Antigen retrieval was performed by heating in sodium citrate solution (10 mmol). Endogenous peroxidase was quenched with 3% (vol/vol) hydrogen peroxide in PBS for 30 minutes. After blocking (5% normal goat serum for 1 hour) the slides were incubated over night with primary antibody (anti-human soluble VEGFR-1, 1:150 dilution). Slides were washed in 1×PBS and exposed to peroxidase-conjugated secondary antibody (1:300, goat anti-rabbit, Vector Laboratories, Burlingame, Calif.) for 45 minutes at room temperature. Finally, avidin biotin complex (Vector Laboratories, Burlingame, Calif.) was applied for 1 hour and staining was detected with the diamino-benzidine chromogen after 5 minutes. Slides were counterstained with haematoxylin. Primary antibody was omitted and replaced by blocking solution in the negative control conditions.

### Enzyme-Linked Immunosorbent Assay (ELISA)

Serum samples from the same women who donated their placentae as well as conditioned media from first trimester villous explants were collected. These were used to determine sFlt-1 expression using an ELISA performed in duplicates according to the manufacturer's instructions (R&D Systems, Minneapolis, Minn.). The minimal detectable concentration was 5 pg/mL of sFlt-1. Protein content in the conditioned media was normalized to the total protein concentration that was measured by Bradford protein assay. The intra assay coefficient of variation of serum samples was not more than 9%. The intra-assay coefficient of variation of the conditioned media samples was 5.7%. One plate of ELISA was used for each experiment.

### Statistics

Statistical analyzes were performed using the SPSS statistical package (SPSS Inc. Chicago, Ill.). Data were analyzed for comparison between means using the Mann-Whitney test and paired or unpaired t-test when applicable. ANOVA was used to analyze the data when more than two groups were analyzed. Significance was defined as P<0.05. Results are expressed as the mean±standard error of the mean (SE).

### Results

Changes in sFlt-1 Expression During Placental Development

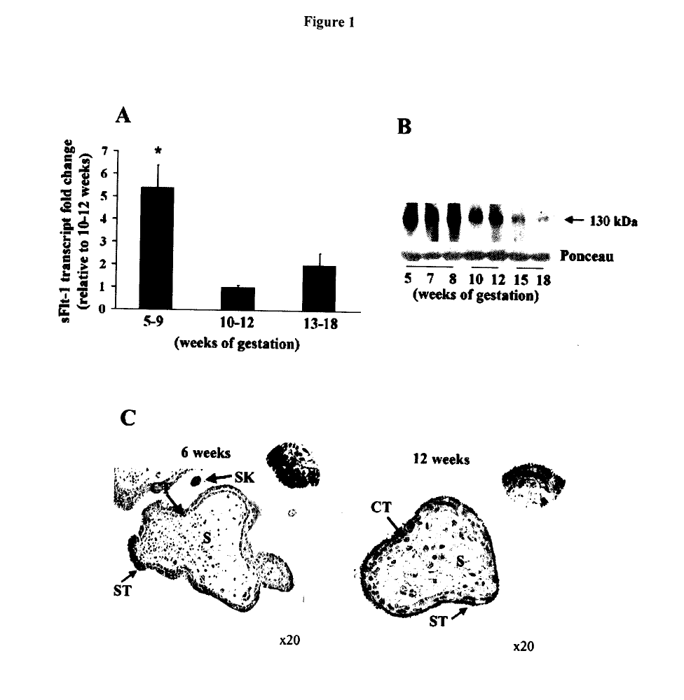

At the end of the first trimester of pregnancy (10-12^{th} week of gestation), when the intervillous space opens to maternal blood, the placenta experiences a surge in oxygenation (Jauniaux E, 2000). As such, whether sFlt-1 expression is affected by this physiologic change in placental oxygenation in vivo was first examined. sFlt-1 transcript expression was significantly increased (5.3±1.4 fold) in early first trimester placental samples (5-9 weeks of gestation) compared to late first trimester (10-12 week) samples (p<0.05) (FIG. 1A). Although an increase in sFlt-1 transcript in samples from 13-18 weeks relative to 10-12 weeks (2 fold) was noted, this increase was not statistically significant (FIG. 1A).

The protein expression of sFlt-1 was examined during normal placental development. Western blot analysis of placental lysate from the same gestational age range showed a decrease in sFlt-1 protein level at 10-12 weeks gestation (FIG. 1B). Similar to the sFlt-1 transcript level, an increase in sFlt-1 protein was also noted at 13-18 weeks of gestation. The spatial localization of sFlt-1 in first trimester samples was assessed using immunohistochemistry. At 6 weeks of gestation, strong positive immunoreactivity for sFlt-1 was noted in the syncytiotrophoblast layer (ST) (FIG. 1C, left panel). After 10 weeks of gestation, reduced immunoreactivity was observed in the ST and restricted mainly to the apical brush border (FIG. 1C, right panel). Strong positive sFlt-1 immunostaining was also observed in syncytial knots in sections of first trimester placental tissue. (FIG. 1C, left panel). No sFlt-1 staining was observed in stromal regions.

sFlt-1 Expression is Increased in High Altitude and Preeclamptic Placentae

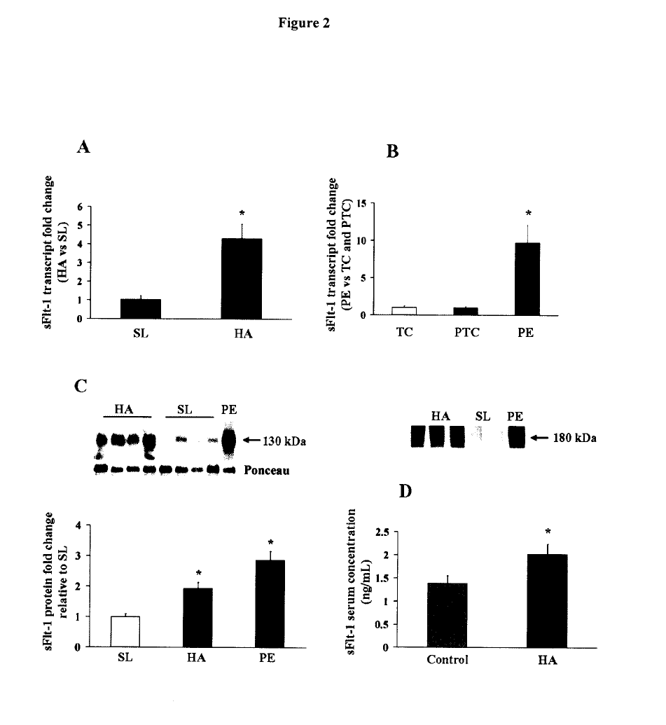

The effect of in vivo conditions of chronic placental hypoxia on sFlt-1 expression was next examined. HA placental tissues showed increased sFlt-1 transcript expression when compared to sea level samples (HA: 4.3±0.7 vs. SL: 1.0±0.2, P<0.05) (FIG. 2A). Early onset severe preeclamptic placental samples, included as an internal positive control, also demonstrated a significant increased sFlt-1 expression compared to normotensive preterm age-matched control samples and term controls (PE: 9.7.0±2.4 vs. controls: PTC 1.0±0.19, P<0.05; TC 1.0±0.08, P<0.05). sFlt-1 transcript level was similar in the preterm age matched (PTC) control and term controls (TC) (FIG. 2B). The effect of mode of delivery on sFlt-1 expression in TC placentae was next examined by qPCR. No changes in sFlt-1 mRNA expression were observed in placentae tissue collected after cesarean section compared to vaginal deliveries (1±0.15 and 1.05±0.14, respectively). Western blots performed on placental lysates from high altitude showed higher sFlt-1 protein expression compared to sea level samples corroborating the transcript levels (FIG. 2C, upper panel). Densitometric analyses showed increased sFlt-1 expression in high altitude samples relative to sea level control tissues (FIG. 2C, lower panel) (1.9±0.2 vs. 1.0±0.1 respectively, P<0.05). Similar to the sFlt-1 protein expression in HA placental tissues, measurements of circulating sFlt-1 in serum of HA patients relative to lower altitudes (control), demonstrated a 45% greater concentrations (HA: 2018±225 vs. control: 1392±159 pg/mL; P=0.01) (FIG. 2D). sFlt-1 concentrations (Y) fell as the time past delivery (X) increased (best fit regression=y=0.0033x²−4.25x+1906R²=−0.92, P=0.009, data not shown). In conjunction with the placental expression of sFlt-1 reported here, the elevated circulating concentrations in high altitude pregnancy appear to be largely of placental origin. Values obtained 3-6 months after delivery in the same women did not differ between low (272±85 pg/mL) and high (305±138 pg/mL) altitude (P=0.42). sFlt-1 concentrations were not related to placental weight at either altitude, hence differences in placental mass do not account for the elevated values at high altitude.

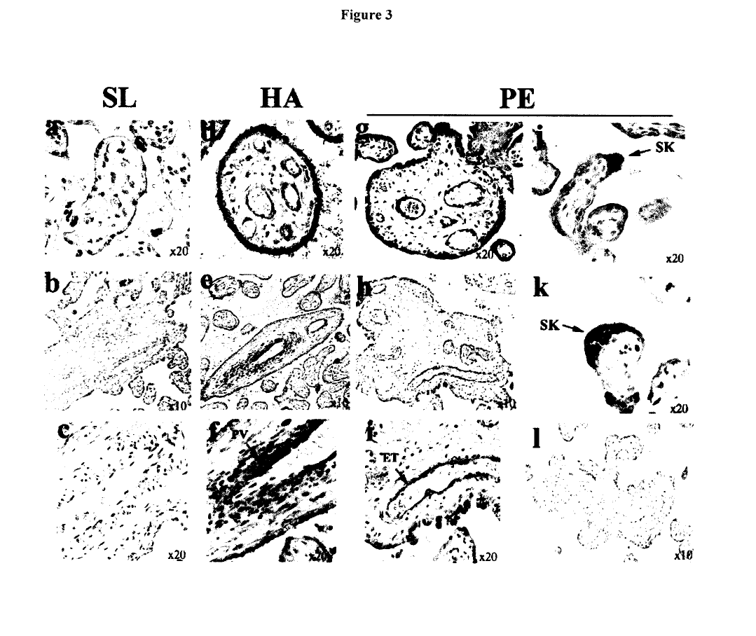

The spatial localization of sFlt-1 in HA, PE and control placental tissues was examined. (FIG. 3). IHC analysis showed strong positive immunoreactivity for sFlt-1 in ST layers and in vascular and perivascular regions of section from HA samples (FIGS. 3 d,e,f). Low/absent staining for sFlt-1 was noted in sea level samples (FIGS. 3 a,b,c). Increased ST staining as well as vascular staining was also observed in preeclamptic samples (FIGS. 3 g,h,i). Strong positive immunoreactivity for sFlt-1 was also consistently observed in syncytial knots mainly in PE samples (FIGS. 3 j and k). No immunoreactivity was observed in control PE section where primary antibody was omitted (FIG. 31).

Hypoxia but not Hypoxia-Reoxygenation, Increases Expression of sFlt-1

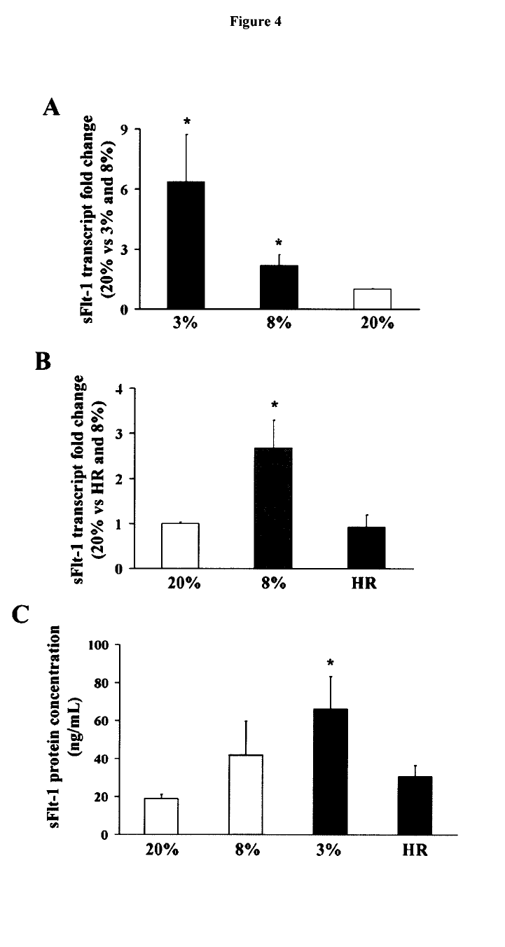

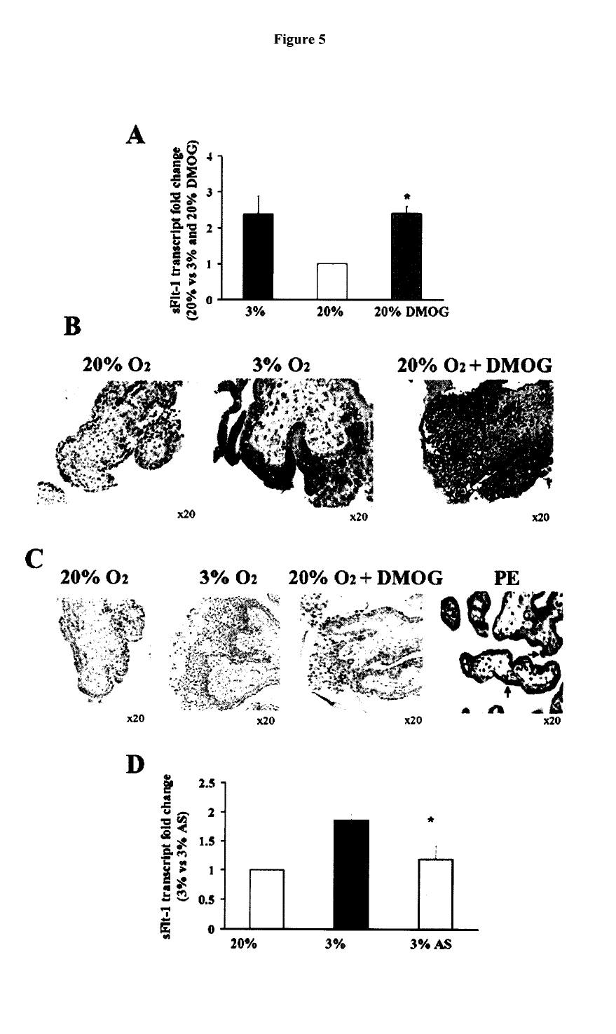

The effect of varying oxygenation on sFlt-1 expression in vitro was examined using explant cultures. Exposure of villous explants to 3% oxygen resulted in increased sFlt-1 mRNA levels when compared to explants cultured at 20% (6.3±2.3 vs. 1.0±0.02, P<0.05) (FIG. 4A). Exposure to 8% O₂ resulted also in a significant increase in sFlt-1 expression (2.2±0.5 vs. 1.0±0.02). Next, sFlt-1 protein expression and localization in explant tissue sections was examined using IHC. Sections of explants exposed to 3% oxygen showed increased sFlt-1 staining in ST layer and proliferating extravillous trophoblast cells when compared to sections from explants exposed to 20% oxygen (FIG. 5B, left and middle panels).

To determine if intermittent change in oxygenation affects sFlt-1 expression, explants were next exposed to hypoxia-reoxygenation (HR) conditions. sFlt-1 transcript level was significantly higher in control explants exposed to 8% than those treated by HR (2.6±0.6 vs. 0.9±0.2, P<0.05) (FIG. 4B). Moreover, sFlt-1 transcript level was not changed following HR relative to standard 20% oxygen condition (0.9±0.2 vs. 1±0.03 respectively). Using ELISA, it was confirmed that the expression of secreted sFlt-1 in conditioned media of explants exposed to 3% and to a lesser extent to 8% oxygen was increased relative to explants maintained in 20% O₂. In contrast, HR exposure did not change sFlt-1 levels compared to 20% O₂ controls (FIG. 4C).

sFlt-1 Expression in Low Oxygen is Mediated via HIF-1

Increased expression of sFlt-1 occurs where oxygen tension is lower both in in vivo and in vitro models, however it is still unclear by what mechanism(s) of hypoxia may regulate the expression of sFlt-1. The impact of HIF-1 stabilization and knockdown on sFlt-1 expression was investigated. DMOG, an indirect stabilizer of the oxygen labile moiety of HIF-1 (Jaakkola P, 2001), was added to the media of explants cultured under 20% oxygen. The sFlt-1 mRNA expression of the DMOG-treated explants was significantly greater than that of explants kept at 20% O₂ and was equivalent to control explants exposed to 3% O₂ (2.4±0.2 vs. 1.0±0.03, respectively, P<0.05), (FIG. 5A). As well, addition of DMOG to the 20% O₂ treated explants resulted in increased sFlt-1 protein expression in all trophoblast layers including EVT when compared to explants exposed to 20% O₂ in the absence of DMOG (FIG. 5B, right and left panels). DMOG-treated explants also exhibited the typical low oxygen-induced outgrowth compared to control explants maintained at 20% O₂ and TUNEL assays indicated that the observed phenotype, due to either 3% O₂ exposure or DMOG treatment, was not likely due to an increased incidence of apoptosis (FIG. 5C).

Finally, using a previously established antisense knockdown technique for HIF-1α (5) it was demonstrated that antisense-treated explants under conditions of reduced oxygenation (3% O₂) have significantly lower expression of sFlt-1 transcript relative to control cultures exposed to the same oxygenated environment in the presence of control sense or medium alone (1.19±0.22 vs. 1.86±0.1, respectively, P<0.05) (FIG. 5D).

### Discussion

The present study has demonstrated that, 1) reduced oxygenation in in vivo and in vitro placental hypoxia causes increased expression of sFlt-1, 2) low oxygenation as opposed to hypoxia-reoxygenation is the driving force for increased sFlt-1 production in human placental tissues, and 3) sFlt-1 expression under conditions of reduced oxygenation is mediated by HIF-1.

Previous studies have reported that low oxygen conditions increase sFlt-1 expression in both primary cytotrophoblast cells as well as in term placental villous explants (Ahmad S, 2004; Nagamatsu T, 2004). The expression profile of sFlt-1 during the first trimester indicates an inverse correlation of this soluble receptor with increasing physiological placental oxygenation occurring after the 10^{th} week of gestation. Hence, early on, when oxygen tension is relatively low, sFlt-1 transcript and protein are elevated. The fine-tuning of sFlt-1 expression during this critical developmental period highlights the importance of this soluble receptor in controlling the effects of its ligand VEGF, which is known to be highly expressed at this time (Kaufmann P, 2004). It is plausible that during early pregnancy, this soluble receptor, by antagonizing VEGF and PIGF function, may temporally restrict early development of the placental microvasculature, thereby diminishing the detrimental impact of early oxygenation (<10 wks), known to be associated with spontaneous miscarriage (Hempstock J, 2003; Jauniaux, 2000). Beyond the critical early period of hypoxia and embryogenesis, subsequent to increased placental blood flow, a decline in sFlt-1 expression may allow vascular growth factors to increase placental vascularity in accordance with the needs of the developing fetus (Kaufmann, 2004).

Placentae from high altitude pregnancies exhibit significant morphological adaptation to chronic hypoxia, including increased vascularity of mature intermediate and terminal villi, resulting in reduced diffusional barrier (vasculosyncytial membrane) and increased density of terminal villi (Zamudio, 2003), The findings suggest that even modestly reduced oxygen tension, estimated at ˜20% reduction relative to sea level in the high altitude placentae (Zamudio, 2003) correlates with increased expression of sFlt-1. It is quite possible that the excess sFlt-1 may function to restrict excessive peripheral vascular development under high altitude conditions, an incomplete adaptation, as a greater incidence of chorangiomas is noteworthy in high altitude placentae (Benirschke, 1999). The systemic effects of chronic hypoxia on sFlt-1 are also clinically significant. High altitude residents are at 2-4 fold greater risk for the development of preeclampsia (Palmer, 1999). Under reduced oxygenation trophoblast cells may secrete more sFlt-1 than its ligands (Nagamatsu, 2004). Hence, increased sFlt-1 expression in normotensive high altitude patients may explain the increased susceptibility to both preeclampsia and intrauterine growth restriction within this population.

Finally, it was found that the primary sources of increased sFlt-1 expression in high altitude placentae are the vascular and perivascular tissues. While the endothelial expression of sFlt-1 has been described in other systems (Hornig, 2000), the data provide the first evidence demonstrating a differential spatial vascular expression between normal (high altitude perivascular expression) and pathologic (preeclampsia syncytiotrophoblast expression) human placentae. It is therefore plausible that while increased sFlt-1 expression in trophoblast layers likely contributes to the elevated serum levels found in preeclampsia and high altitude, the increased sFlt-1 vascular expression may be responsible for the high sFlt-1 levels in the fetal circulation of preeclamptic pregnancies (Tsao, 2005).

Mode of delivery and preterm birth are two factors that can affect gene expression in placental tissue and may be confounding factors. It has been shown that during vaginal deliveries that are accompanied by birth asphyxia there is increased expression of VEGF, Flt-1 and VEGFR-2 in placental tissue (Trollman, 2003). The results show that there is no difference in sFlt-1 expression between normal vaginal deliveries and cesarean section, implying that the process of normal labor do not trigger sFlt-1 expression. Deliveries were not included that were complicated by birth asphyxia that may be related to placental hypoxia, which can potentially lead to increased sFlt-1 expression. The findings here support that sFlt expression is stable during the third trimester of pregnancy and can be explained by the stable oxygen level during this period. Consequently, both mode of delivery or gestational age at the time of delivery did not confound the results.

The in vitro model of placental hypoxia using first trimester explants supports previous reports demonstrating increased sFlt-1 expression in hypoxic conditions in vitro both in primary isolated first trimester trophoblast cells as well as in term explants (Ahmad, 2004, Nagamatsu, 2004). The stimulatory effect of lowered oxygen tension on sFlt-1 expression has been postulated to be mediated by HIF-1 (Li, 2005). In silico analysis of the Flt-1 gene, which is the precursor of sFlt-1, revealed hypoxia responsive elements (HRE) in its promoter region (Gerber, 1997), supporting that sFlt-1 may be regulated by HIF-1. The experimental findings of increased sFlt-1 expression under DMOG-mediated HIF-1 stabilization or its decreased expression using HIF-1α knockdown under reduced oxygenation provides direct evidence that HIF-1α regulates sFlt-1 transcript expression. Previous studies indicate that HIF-1α expression is increased in preeclamptic (Caniggia, 2002, Nagamatsu, 2004) and high altitude placentae (Caniggia, 2002) and therefore may explain the increased sFlt-1 expression seen in these conditions.

The in vitro studies demonstrate that sFlt-1 expression does not change after hypoxia-reoxygenation. Cycles of hypoxia followed by reoxygenation has been proposed as the underlying condition in preeclampsia that induces oxidative stress thus causing placental damage (Hung, 2002). HR was specifically tested because both VEGF in rat myocardium (Sasaki, 2000) and TNFα in placental explants (Hung, 2004) are elevated following an HR insult and associated with increased secretion of sFlt-1. Although HR may induce oxidative stress in the human placenta (Hung, 2001), the data here, the epidemiological data (Zamudio, 2003), and the recent data on aberrant global placental gene expression in the same in vitro and in vivo models tested here (Soleymanlou, 2005) suggests that chronically reduced oxygen may be the main trigger. While it is shown that low oxygen tension contributes to aberrant global placental gene expression in early onset severe preeclampsia (Soleymanlou, 2005), in preeclamptic placentae sFlt-1 expression was significantly more elevated than that of high altitude placentae when compared to controls, suggesting that other pathways may potentially be involved in regulating sFlt-1 expression in preeclampsia.

Preeclampsia is also characterized by excessive shedding/turnover of trophoblast microfragments and syncytial knots into maternal peripheral circulation, an event that has been hypothesized to contribute to generalized endothelial dysfunction (Sargent, 2003). Increased sFlt-1 protein expression was found in placental syncytial knots, particularly in preeclamptic placentae, suggesting that shed syncytial fragments may serve as a vehicle to carry excess sFlt-1 into the maternal circulation, hence enhancing the detrimental anti-angiogenic function of sFlt-1 on the maternal peripheral vasculature.

In conclusion, increased sFlt-1 expression in the human placenta under low oxygen conditions in vivo and in vitro is mediated by HIF-1α. Chronically low oxygen tension, as opposed to hypoxia-reoxygenation, plays an important role in regulating the expression of this angiogenic antagonist in the human placenta. Increased sFlt-1 expression in high altitude placentae could explain the greater population susceptibility to the development of preeclampsia in this environment.

### EXAMPLE 2

### sFlt-1 Expression is Increased in Placentae from IUGR Pregnancies

Elevated expression of soluble VEGF receptor-1 (sFlt-1) in preeclampsia plays a critical role in the pathogenesis of this serious disorder of pregnancy. Low oxygen has been shown to be a critical factor responsible for increased sFlt-1 expression in the human placenta. Intrauterine growth restriction (IUGR) is characterized by decreased placental perfusion and increased risk for developing preeclampsia. The objective of this study was to examine sFlt-1 expression in different models of pregnancies complicated by IUGR.

Methods: Placentae from four subgroups were collected: early severe IUGR with abnormal umbilical and uterine artery Doppler (IUGR, n=16), small for gestational age with normal Doppler (SGA; n=8), severely discordant dichorionic and monochorionic twins with abnormal umbilical artery Doppler (n=6) and age matched normal singletons and twins controls (C; n=13 respectively). sFlt-1 mRNA expression was measured by real time PCR analysis using specific TaqMan primers and probes. Protein expression was measured by Western Blot and immunohistochemical analysis using a polyclonal antibody against sFlt-1.

Results: sFlt-1 transcript levels were significantly increased in IUGR placentae compared to normal controls (2.66 fold). Increased sFlt-1 mRNA expression was also found in samples obtained from random multiple sampling from the same IUGR and control placentae. SFlt-1 transcripts did not show any differences in SGA placentae. In contrast, sFlt-1 mRNA expression was significantly higher in the small IUGR twin placentae from the discordant twin pregnancies (2.96 fold) relative to the normal co-twin. Western blot analysis showed increased sFlt-1 protein expression in severe IUGR placentae relative to normal controls. Immunohistochemical analysis revealed strong positive immunoreactivity for sFlt-1 in both IUGR placentae from singletons and twins, mainly localized to the trophoblast layers.

Conclusions: sFlt-1 expression is increased in IUGR placentae with abnormal umbilical artery Doppler of singletons and also in discordant IUGR twins. Reduced placental perfusion leading to placental hypoxia, may contribute to the increased expression of sFlt-1 in IUGR pregnancies. Elevated sFlt-1 expression may explain the increased rate of preeclampsia found in severe IUGR pregnancies.

### EXAMPLE 3

### Smads Signaling in Normal and Preeclamptic Placentae

Members of the transforming growth factor β (TGFβ) superfamily play an important role in a variety of biological processes including cell proliferation, differentiation, migration and apoptosis. During placental development, TGFβs are known to regulate cellular events associated with trophoblast differentiation/invasion. TGFβ₃ expression is elevated in placentae from pregnancies complicated by preeclampsia, and increased levels of circulating TGFβ₁ have been associated with increased risk of preeclampsia. Smad proteins are intracellular signaling mediators of the TGFβ pathway. Upon ligand induction, the TGFβ type II receptor recruits and activates the type I receptor, which in turn phosphorylates and activates the Receptor-regulated Smads (R-Smads) leading to the propagation of the TGFβ signaling. Conversely, Inhibitory Smads (I-Smad) act to prevent the signal transduction of the TGFβ pathway. Although TGFβs are important in the human placenta, intracellular signaling via Smads remains elusive. The objective of this study is to examine the expression/activation of Smad2 (R-Smad) and Smad7 (I-Smad) during normal placental development and in placentae from preeclamptic pregnancies.

Methods: Protein lysates from human placental tissues across gestations (6-40 weeks) and from early onset (25-30 weeks) and late onset (37-40 weeks) preeclampsia were subjected to SDS/PAGE and Western Blotting using rabbit polyclonal antibodies that specifically recognize human Smad2, Smad7 and phospho-Smad2.
Results: Although during normal placentation, total Smad2 protein expression was detected throughout gestation, its expression profile exhibited a peak at around 11-18 wks of gestation and markedly decreased thereafter. Smad2 was active as indicated by phospho-Smad2 during early gestation (6-8 wks) while little activity was noted at term. In contrast, expression of Smad7 was very low at early stage of gestation and increased near term. Of clinical significance, phosphorylation of Smad2 was notably increased in both early and late onset preeclampsia. While a marked increase in Smad7 content was observed in placentae from early onset preeclampsia, no changes in Smad7 levels were found in late onset preeclamptic tissue.
Conclusions: During placental development expression of Smad2 and Smad7 is temporally regulated. The inverse correlation of the level of phospho-Smad2 and Smad7 suggests an activation of the TGFβ signaling pathway during early gestation and an inhibition of the pathway during late gestation. In early onset preeclampsia, the increase of both phospho-Smad2 and Smad7 implicates an autocrine regulation by TGFβ, in which activation of TGFβ signaling leads to an increased expression of Smad7. However, in late onset preeclampsia, activation of Smad2 is not accompanied by an increase of Smad7, indicating a different regulatory mechanism of TGFβ signaling.

### EXAMPLE 4

### Mechanisms of Oxygen Sensing in Intra Uterine Growth Restriction Placentae

In virtually all mammalian systems including the human placenta, the cellular answers to different oxygen tension are largely mediated by transcription factors termed Hypoxia Inducible Factors (HIFs). A well-conserved family of prolyl hydroxylases enzymes (PHD1, PHD2, PHD3) is important in the post-translational modification of HIF-1α, which is required for the degradation of HIF-1β via the ubiquitin-proteasome pathway. Emerging evidence has implicated Seven In Absentia Homolog 1 and 2 (SIAH1/2) molecules as oxygen sensors and regulator of PHDs expression. SIAHs may function as upstream oxygen sensitive regulators of PHD expression by regulating the ubiquitin-proteasomal degradation of these proteins. Although it is well established that oxygen via HIF-1 plays a key role in normal placental development and in placental pathologies, the pathways by which trophoblast cells sense oxygen remain elusive. The objective of the present study was to examine oxygen sensing mechanisms, in particular SIAHs and PHDs expression, in placentae from normal pregnancies and pregnancies complicated by severe intrauterine growth restriction (IUGR).

Methods: Human placental tissue from early severe IUGR pregnancies (>3rd percentile, n=13, 31.5+1.2 weeks, birth weight 1025±228 grams) with absence of end diastolic flow (AEDF), and age-matched control placentae (C, n=13, 31+2.5 weeks, birth weight 1494±552 grams) were snap frozen immediately after delivery. Samples were processed for real-time PCR analysis using specific TaqMan probes for SIAH1/2, PHD1, PHD2 and PHD3. Immunoblot analysis was performed using PHD1, 2 and 3 as well as SIAH1/2 specific antisera. PHDs ubiquitination was investigated by immunoprecipitation of PHDs followed by immunoblotting using anti-ubiquitin Mab.
Results: Real-time PCR analysis showed significantly increased PHDs transcripts in IUGR placentae compared to control tissue. Surprisingly, immunoblot analysis demonstrated no difference in PHDs protein expression relative to control tissues. Consistent with these protein findings, ubiquitination of PHDs was significantly increased in IUGR placentae compared to controls. To further elucidate the regulatory mechanism of PHD degradation, the expression of SIAH1 and SIAH2 was examined. Transcript levels of both SIAH1 and SIAH2 were significantly increased in IUGR placentae suggesting that these molecules may be involved in the increased degradation of PHD's found in IUGR.
Conclusion: These data provide molecular evidence for enhanced low oxygen sensing in severe IUGR placentae.

### EXAMPLE 5

### Mechanisms of Oxygen Sensing in Placentae from High Altitude Pregnancies

Hypoxia Inducible Factor 1 (HIF-1) stimulates the expression of a variety of genes involved in physiological response to hypoxia. Under normoxic condition, the HIF-1α subunit is recognized by the von Hippel-Lindau protein (VHL) and targeted for proteasome-mediated proteolysis. A prerequisite for binding to VHL is the hydroxylation of HIF-1α by prolyl-hydroxylases, termed PHD1, 2 and 3. PHDs are also O₂ sensors, as molecular O₂ is a substrate for their reaction. Recent evidence indicates that the E3 ligases Seven In Absentia Homologues 1 and 2 (SIAH1-2) target PHDs for ubiquitination and proteasomal degradation, thereby regulating their activity. In addition, SIAHs also function as oxygen sensors as varying oxygen levels regulate their expression. HIF-1α expression is increased in high altitude (HA) placentae, a unique model of physiological adaptation to chronic hypoxia. The objective of this study was to examine the expression of SIAHs and their potential role on PHDs regulation in HA pregnancies.

Materials and Methods: Placental tissue from high altitude (Leadville, 3100 m; n=24) and control sea-level pregnancies (Toronto, n=14) were collected and snap-frozen immediately after delivery. PHD1, 2, 3 and SIAH1, 2 protein levels were determined by Western blotting using specific polyclonal antibodies. SIAHs mRNA levels were assessed by real-time PCR. PHDs ubiquitination was investigated by immunoprecipitation of PHDs followed by immunoblotting using anti-ubiquitin Mab.

Results: A decrease in SIAH1 transcripts was observed in placentae from HA compared to sea level (SL). Consistent with this result, SIAH1 protein content in HA placentae was significantly lower than that of SL placentae (p<0.05). No changes in SIAH2 mRNA and protein levels were detected in HA vs. SL placentae. PHDs mRNA expression is elevated in HA placentae implicating a role for these enzymes as oxygen sensors. It was found that PHD2 and 3 protein levels were significantly decreased in HA placentae when compared to SL, while no changes in PHD1 protein level were observed. Consistent with these protein findings, PHD1 ubiquitination was significantly decreased while that of PHD2 and to a lesser extent PHD3 was increased in HA compared to SL samples.
Conclusions: Both SIAHs and PHDs play an important role in oxygen sensing in HA pregnancies. PHDs are differentially expressed and regulated in HA placentae, implicating a different role for these enzymes in regulating HIF-1α expression in response to chronic hypoxia.

### EXAMPLE 6

### Novel Role of Mtd in Trophoblast Cell Fate

Recently, a number of apoptotic molecules have been found to be involved in other cell biological functions, including cell cycle regulation. Herein a possible dual role of Mtd/Bok, a pro-apoptotic member of the Bcl-2 family, in the regulation of human trophoblast cell death and proliferation was investigated. The objective of this study was to examine the temporal and spatial pattern of expression of Mtd in human placentae during the first trimester of gestation and to examine whether changes in Mtd expression were associated with cellular events other than apoptosis.

Methods: Spatial and temporal localization of Mtd was determined by fluorescence immunohistochemistry. First trimester placental sections were double stained using a polyclonal antibody that recognizes all isoforms of Mtd in conjunction with markers of either proliferation (Ki67 and PCNA), or cell death (active caspase-3, TUNEL). Laser Capture Microdissection was used to isolate extravillous trophoblast cells (EVT) within the anchoring villi from placental section of 5-12 weeks gestation. Laser captured samples were subjected to real time PCR analysis to enable characterization of Mtd isoform specific expression.
Results: The expression of Mtd changes both spatially and temporally during the first trimester. In floating villi of early first trimester tissue (5-8 weeks) Mtd is localized to the cytotrophoblast cells whereas in later first trimester (12-15 weeks) the expression of Mtd switches to the apical border of the syncytiotrophoblast layer. Furthermore, early on, Mtd expression is found in the EVT cells forming the anchoring villi, while later on it is preferentially localized to the distal part of the EVT columns. Both Mtd-L and Mtd-P transcripts were expressed in trophoblast cells of both anchoring and floating villi, however, the relative abundance of Mtd-L was greater than that of Mtd-P. Preliminary data indicated that the levels of Mtd mRNA correlated to the pattern of Mtd protein expression. Both Mtd-L and Mtd-P transcripts were highly expressed at 5-8 weeks in the trophoblast layers and their expression decreased with advancing gestation. As well, early in first trimester, both isoforms were distributed throughout the column and as gestational age advanced the expression of both Mtd-L and Mtd-P became predominantly restricted to the distal column. Immunohystochemical analysis revealed a strong co-localization of Mtd with markers of proliferation (Ki67 and PCNA) in both chorionic villous “stem” cytotrophoblast cells as well as in EVT cells forming the proximal column of the anchoring villi. In addition, cells that were undergoing apoptosis were also positive for Mtd, however apoptosis occurred only sporadically, being found almost exclusively in the EVT cells. Interestingly, the pattern of Mtd in proliferating cells (punctuate) was characteristically different from that found in cells undergoing apoptosis (aggregated) suggesting that Mtd may use different mechanisms to regulate various stages of cell fate.
Conclusion: Mtd not only regulates cell death, as previously determined, but Mtd-L and to a lesser extent Mtd-P may also be involved in regulating the cell cycle of trophoblast cells during early placental development.

### EXAMPLE 7

### Summary

The expression and functional regulation of Mcl-1, a Bcl-2 family member, was examined in human models of physiological and pathological placental oxygenation. Only early-onset preeclamptic placentae (PE) were associated with cleavage of death-suppressing Mcl-1L and a switch in Mcl-1 isoform expression towards cell death-inducer Mcl-1S. Mcl-1L cleavage, induced by hypoxia-reoxygenation in human villous explants, was mediated by caspase-3, as pharmacological caspase inhibition (z-VAD-fmk or z-DEVD-fmk treatments) prevented this cleavage. Altitude-induced chronic hypoxia, tilted Mcl-1 expression towards the death suppressing Mcl-1L isoform, which was accompanied by a reduction of apoptotic markers (cleaved caspase-3 and -8). These changes in Mcl-1 isoform expression were accompanied by decrease in trophoblast cell fusion in both physiological and pathological placental hypoxia as determined by syncytin expression. Hence, while both pathological and chronic placental hypoxia are associated with slowed trophoblast differentiation, trophoblast apoptosis is only upregulated in PE, possibly due to oxygen-induced switch in generation of pro-apoptotic Mcl-1 molecules. This study provides the first observation of aberrant oxygen-mediated Mcl-1 isoform/cleavage changes associated with human disease.

Mcl-1 expression in pregnancies complicated by preeclampsia was investigated, in particular how conditions of in vitro and in vivo placental hypoxia affect the Mcl-1/Mtd apoptotic rheostat. Additionally, the expression of markers of trophoblast cell death and differentiation were studied in physiological altitude-induced chronic hypoxia and in pathological hypoxic conditions such as preeclampsia. The data provide novel insights into the regulation of Mcl-1 expression by oxygen and the importance of Mcl-1 as a novel regulator of human trophoblast cell death in preeclampsia.

### Materials and Methods

### Tissue Sampling

Collection was in accordance with participating institutions' ethics guidelines. Severe early-onset preeclampsia was diagnosed based on the American College of Obstetrics and Gynecology (ACOG) criteria. Preeclamptic placentae (PE, n=30) and preterm normotensive age-matched control placentae (AMC, n=30) were collected from deliveries at Mount Sinai Hospital. Areas with calcified, necrotic or visually ischemic tissue were omitted from sampling. Subjects suffering from diabetes, essential hypertension, kidney disease or infections were excluded. Pregnant patients with essential hypertension (EH; n=4, at term) and pregnancies affected by IUGR (n=6, gestational age 32-36 weeks with fetal weight less than 5^{th} percentile) without preeclampsia were included as controls. Preterm deliveries were due to multiple pregnancy (30%), preterm labor due to incompetent cervix (40%), premature preterm rupture of membrane (20%) and spontaneous rupture of membranes (10%). All preterm and term control groups did not show clinical or pathological signs of preeclampsia, infections or other maternal or placental disease. First-trimester human placental tissues (6-12 weeks of gestation, n=10) were obtained from elective terminations of pregnancies by dilatation and curettage. High altitude (HA) and moderate altitude (MA, used as control) placental samples (n=16 each) were collected from pregnancies in Leadville (3100 m) and Denver (1700 m), Colorado, USA. HA and MA placentae were obtained from healthy normal vaginal deliveries from term normotensive patients. Fifteen normotensive placentae obtained from term deliveries at sea-level (SL, Toronto) were also included as an additional control with clinical maternal and fetal parameters within normal physiological range. Due to organ heterogeneity and the fact that the level of perfusion is different depending on location within the placenta, multiple specimens were sampled from central and peripheral regions from both maternal and fetal sides of pathologic, high altitude and control placentae. The SL, MA and HA groups did not show clinical or pathological signs of preeclampsia, infection or other placental disease.

Human Chorionic First Trimester Villous Explant Culture and zVAD-fmk and zDEVD-fmk Treatments.

Explant cultures were performed as previously described (Caniggia, 2000). Briefly, placental tissues (5-8 weeks of gestation, 15 sets) were placed in ice-cold PBS and processed within 2 hours of collection. Tissues were aseptically dissected to remove decidual tissue and fetal membranes. Small fragments of placental villi (25-45 mg wet weight) were teased apart, placed on Millicell-CM culture dish inserts (Millipore Corp., Bedford, Mass., USA) pre-coated with 0.15 mL of undiluted Matrigel (Collaborative Biomedical Products, Bedford, Mass., USA), and transferred to a 24-well culture dish. Explants were cultured in serum-free DMEM/F12 (Gibco BRL, Grand Island, N.Y., USA) supplemented with 100 μg/mL streptomycin, 100 U/mL penicillin, and incubated overnight at 37° C. in 5% CO₂ in air to allow attachment. Explants were maintained in standard condition (5% CO₂ in 95% air) or in an atmosphere of 3% O₂/92% N₂/5% CO₂ for 48 hrs at 37° C. Explants from more than 10 different placentae in more than 15 separate experiments were used. A minimum of 3 explants per experimental condition was used at all times. Explants were exposed to hypoxia-reoxygenation (H/R) as previously described (Hung, 2002; Soleymanlou, 2005) in presence of 100 μM of the pan-caspase inhibitor z-VAD-fmk (BioMol, Plymouth Meeting, Pa.) or caspase-3-specific inhibitor z-DEVD-fmk (R&D Systems, Minneapolis, Minn.) both dissolved in DMSO (equivalent volume of DMSO in absence of inhibitors was used in control conditions).

### RNA Analysis

RNA extraction was performed using a Rneasy Mini Kit (Qiagen), reverse transcribed using a random hexamer approach, and amplified by 40 cycles of quantitative PCR (15 minutes at 95° C., cycle: 30 seconds at 95° C., 30 seconds at 60° C. and 30 seconds at 72° C.). Southern blot analysis was performed as previously described (Soleymanlou, 2005), using a ³²P-labelled full-length Mcl-1L probe. Primer sequences used in RT-PCR/Southern blot analysis were: Mcl-1 (NM_{—}021960): forward 5′-ATGTTTGGCCTCAAAAGAAACGCG-3′ [SEQ ID NO. 4] and reverse 5′-GGCTATCTTATTAGATATGCCAA-3′ [SEQ ID NO. 5] (Mcl-1L: predicted size=1054 bp and Mcl-1S: predicted size=806 bp) and β-actin (NM_{—}001101): forward 5′-CTTCTACAATGAGCTGCGTG-3′ [SEQ ID NO. 6], reverse 5′-TCATGAGGTAGTCAGTCAGG-3′ [SEQ ID NO. 7] (predicted size=304 bp). All amplified and cloned products were confirmed by sequencing. Quantitative PCR was performed using the SYBR Green I dye DyNamo™ HS kit (MJ Research) based on the manufacturer's protocol using isoform specific primers for Mtd-L and Mtd-P (Mtd-L: Forward 5′-GCCTGGCTGAGGTGTGC-3′ [SEQ ID NO. 8], Mtd-P: Forward 5′-GCGGGAGAGGCGATGA-3′ [SEQ ID NO. 9], Reverse (both L and P) 5′-TGCAGAGAAGATGTGGCCA-3′ [SEQ ID NO. 10], Mcl-1L: Forward 5′-ATGCTTCGGAAACTGGACAT-3′ [SEQ ID NO. 11], Mcl-1S: Forward 5′-CCTTCCAAGGATGGGTTTG-3′ [SEQ ID NO. 12], Mcl-1 reverse (both L and S) 5′-CTAGGTTGCTAGGGTGCAA-3′ [SEQ ID NO. 13]. For syncytin and cytokeratin 7 analyses, qRT-PCR was performed using Assays-on-Demand™ Taqman primers and probe (Applied Biosystems, Foster City, Calif.). Analysis was done using the DNA Engine Opticon®2 System (MJ Research). Data for all qPCR analyses were normalized against expression of 18S ribosomal RNA as previously described (Livak, 2001).

### Western Blot Analysis

Western blotting was performed as previously described (MacPhee, 2001). Fifty μg of total protein form placental tissue or cell line was subjected to 12% (wt/vol) SDS-PAGE. Membranes were probed at 4° C. overnight with a 1:1000 dilutions of primary antibodies:rabbit polyclonal Mtd antibody capable of recognizing all isoforms as previously reported (Soleymanlou, 2005); Mcl-1-specific rabbit polyclonal antibody (SC-819 clone S-19 from Santa Cruz Biotechnology, Santa Cruz, Calif.); specific cleaved caspase-3 (Asp175) (5Al) and cleaved caspase-8 (Asp374) rabbit polyclonal antibodies (Cell Signaling, Beverly, Mass.). For anti-Mtd and Mcl-1 antibodies, pre-immune serum and competing peptides were used as controls. A rabbit polyclonal antibody was generated against N-terminal amino acids (28-41) of syncytin (NM_{—}014590) and pre-immune serum was used as control. After overnight incubation, membranes were washed with TBS/T and incubated for 60 minutes at room temperature with 1:5000 diluted horseradish peroxidase-conjugated anti-rabbit (Santa Cruz Biotechnology). Blots were exposed to chemiluminescent ECL-plus reagent (Amersham, Piscataway, N.J.). All blots were confirmed for equal protein loading at all time using ponceau staining.

### Immunohistochemistry

Immunohistochemical analyses were performed using an avidin-biotin-based immunoperoxidase approach, as previously described (Caniggia, 1999). Nonspecific binding sites was blocked using 5% (vol/vol) normal goat serum (NGS) and 1% (wt/vol) BSA in Tris-buffer. Slides were incubated overnight at 4° C. with a 1:200 dilution of rabbit polyclonal anti-Mtd or anti-Mcl-1 antibodies. After washing, slides were probed with 300-fold dilution of biotinylated goat anti-rabbit or goat anti-mouse IgG (Vector Laboratories, Burlingame, Calif.) for 1 hour at 4° C. Avidin-biotin complex was applied for 1 hour. Slides were developed in 0.075% (wt/vol) 3,3-diaminobenzidine in PBS (pH 7.6) containing 0.002% (vol/vol) H₂O₂, giving rise to a brownish product. Slides were counterstained with hematoxylin, dehydrated in an ascending ethanol series, cleared in xylene, and mounted. In control experiments, primary antibodies were replaced with blocking solution (5% [vol/vol] NGS and 1% [wt/vol] BSA).

### Statistical Analysis

Data are represented as mean±SEM of at least 3 separate experiments carried out in triplicate. Statistical difference was determined by Student's t test for paired groups, Significance defined as P<0.05.

### Results

### Mcl-1 Transcript and Protein Expression in Preeclampsia

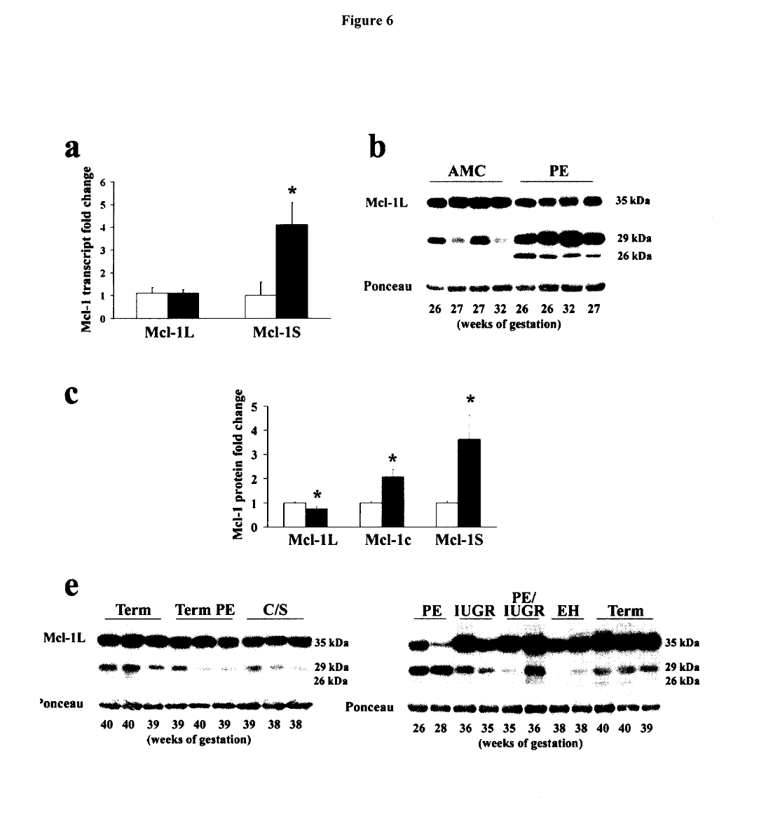

The placental transcript and protein expression of Mcl-1 was first assessed in placentae of severe early-onset preeclamptic patients (PE) relative to age-matched controls (AMC). Quantitative real-time PCR (qRT-PCR) using isoform-specific primers for anti-apoptotic Mcl-1L and pro-apoptotic Mcl-1S demonstrated that Mcl-1L expression was unchanged between PE and AMC, while Mcl-1S expression was significantly increased in PE (4 fold, p=0.001) relative to AMCs (FIG. 6a). RT-PCR followed by hybridization with a ³²P-labeled Mcl-1 specific probe confirmed the increased mRNA expression of Mcl-1S in PE relative to AMC (data not shown). Sequence analysis of the PCR products confirmed identity of MCl-1L and Mcl-1S (data not shown).

Western blot analyses showed noticeable changes in Mcl-1 protein expression between AMC and early onset PE. Mcl-1L protein (Mᵣ˜37 kDa) was prominent in AMC tissues, while its content was markedly decreased in PE samples (representative blot, FIG. 6b). In addition, 2 shorter Mcl-1-immunoreactive bands were observed, both of which appeared to be stronger in PE tissues (FIG. 6b). These short Mcl-1 immunopositive bands migrated at the relative molecular weights corresponding to 29 kDa and 26 kDa, respectively. To quantify changes in content of various immunoreactive Mcl-1 proteins, densitometric analysis was performed. Protein expression of Mcl-1L, Mcl-1 p29 and Mcl-1S (previously reported as the 26 kDa band (Ref)) was decreased by 25% (p=0.01), increased by 2-fold (p=0.003) and increased by 3.6-fold (p=0.01), respectively, in placentae of early onset PE patients relative to normotensive age-matched controls (FIG. 6c).

The Mcl-1 protein expression profile was also examined in late-onset preeclamptic placentae relative to normal term and elective caesarian section (C/S) deliveries. No significant differences were observed in Mcl-1L expression between late preeclampsia relative to normal term deliveries and non-labour C/S deliveries (FIG. 6d). Other Mcl-1 immunoreactive isoforms such as p29 and Mcl-1S were hardly noticeable in these term placentae. When Mcl-1 protein expression was compared between early-onset PE placentae and other placental pathological controls (IUGR pregnancies, late-onset preeclamptic associated with IUGR, patient suffering from essential hypertension and normal term tissues), p29 Mcl-1 and Mcl-1S isoforms were present exclusively in the placentae of patients with the early severe form of this disease (FIG. 6e).

### Mcl-1 Cleavage in Human Villous Explants is Regulated by Caspase Activation.

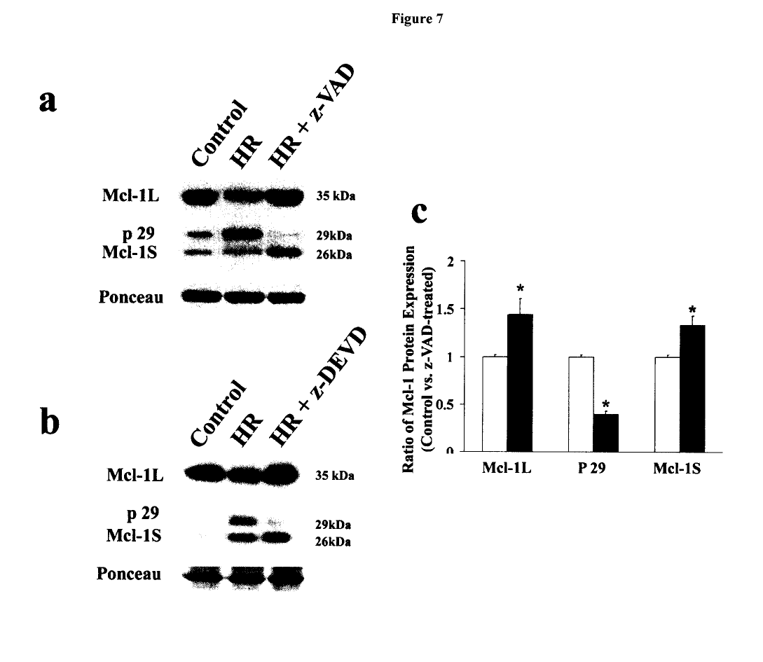

In order to investigate whether the p29 Mcl-1 immunoreactive band can be due to caspase-mediated cleavage, first trimester placental explants were exposed to conditions of hypoxia-reoxygenation (H/R). H/R was chosen as it is a known inducer of caspase activation and trophoblast apoptosis in vitro (Hung, 2002; Soleymanlou, 2005). Explants were exposed to H/R in presence or absence of zVAD-fmk, a broad-based inhibitor of caspase activity and inhibitor of caspase-mediated Mcl-1L cleavage (Weng, 2005; Snowden, 2003; Herrant, 2004; Han, 2004). Relative to untreated controls, tissues exposed to H/R demonstrated a notable Mcl-1 isoform switch i.e. Mcl-1L protein decreased with a concomitant increase of p29 immunoreactive Mcl-1 protein (FIG. 7a). Exposure of H/R-treated explants to zVAD-fmk abrogated the appearance of the immunoreactive p29 Mcl-1 band and restored Mcl-1L content as well as increased Mcl-1S amount relative to H/R and untreated tissues. The latter suggests that Mcl-1S may also be regulated by caspase cleavage. To validate whether Mcl-1 cleavage under H/R was mediated by caspase-3/7, explants under H/R were incubated in presence and absence of caspase-3/7 specific inhibitor zDEVD-fmk. Similar to the observations made with zVAD-fmk, zDEVD-fmk treatment prevented caspase mediated Mcl-1c formation from Mcl-1L under H/R conditions (FIG. 7b). Also, Mcl-1S was increased by the zDEVD-fmk treatment. Thus, both Mcl-1L and Mcl-1S appear to be regulated by caspase cleavage under H/R stress and p29 Mcl-1 is most likely the previously described pro-apoptotic Mcl-1 cleavage product (Weng, 2005; Snowden, 2003; Han, 2004; Han, 2005; Michels, 2004), designated as Mcl-1Lc from here on.

### Oxygen Regulation of Mcl-1 in First Trimester Human Placental Tissues

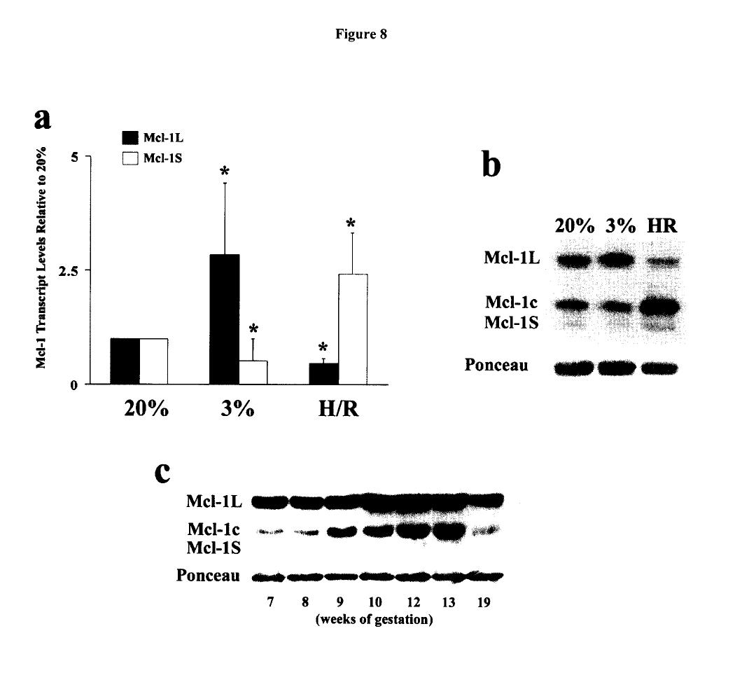

As preeclampsia is associated with aberrant utero-placental oxygenation resulting in placental hypoxia/oxidative stress, effects of varying oxygenation on Mcl-1 expression were assessed in vitro using villous explants exposed to 20% oxygen, 3% oxygen and H/R. In 3% oxygen relative to standard 20% O₂ conditions, mRNA expression of Mcl-1L significantly increased (over 2.5-fold, p=0.0095) while that of Mcl-1S decreased (0.2-fold, p=0.01) (FIG. 8a). The opposite expression profile was observed under H/R conditions where Mcl-1L decreased (2-fold, p=0.01) and Mcl-1S increased (2.5-fold, p=0.01) relative to 20% control (FIG. 8a). The protein profile of Mcl-1 under 3% oxygen, showed increased Mcl-1L expression relative to 20% O₂ controls (representative blot, FIG. 8b). Importantly, H/R resulted in markedly decreased levels of Mcl-1L and a concomitant increased formation of Mcl-1c (p29 Mcl-1) relative to 20% O₂ (FIG. 8b). Although the expression of Mcl-1S at the transcript level was markedly increased in H/R relative to 20% O₂, this pattern was not evident at the protein level. This could be explained by the fact that Mcl-1S, being a low abundant isoform (relative to the L isoform), is rapidly degraded/cleaved at the protein level due to caspase activation under H/R conditions.

During first trimester, the human placenta experiences a surge in oxygenation around 10-12 weeks of gestation when the intervillous space opens to maternal circulation after dislocation of endovascular plugs. Rapid placental oxygenation, similar to a reperfusion injury (from 15-20 mmHg before 8 weeks to 55-60 mmHg at the initial stages of intervillous perfusion), may hence result in a transient state of oxidative stress. Therefore, Mcl-1 expression was examined in placental samples from first and second trimester gestations and increased formation of Mcl-1Lc (p29 Mcl-1) was observed at 10-13 weeks (coinciding with a placental surge of oxidative stress), which declines at later gestations upon placental adaptation to the this initial perfusion insult (FIG. 8c).

### Mtd and Mcl-1 Expression in In Vivo Chronic Placental Hypoxia.

Preeclamptic placentae have a global gene expression similarity to placental tissues obtained from high altitude pregnancies, as both conditions may be affected by aberrant placental oxygenation (Soleymanlou, 2005). As such, expression of Mcl-1 and its death-inducing interacting partner Mtd, was studied under physiologically reduced placental oxygenation using high altitude placentae (HA).

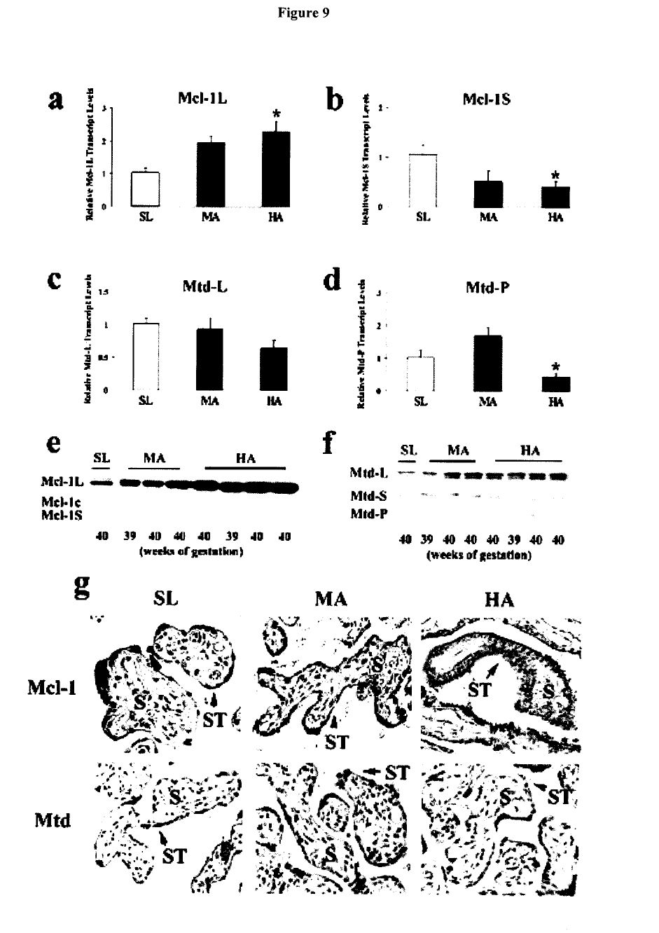

Placental Mcl-1 isoform mRNA expression was different between HA and controls [moderate altitude (MA) and normal sea-level placentae (SL)]. While Mcl-1L transcripts were significantly increased (˜2-fold, p<0.05) in HA and MA relative to SL, Mcl-1S transcript levels were significantly decreased in HA and MA relative to SL samples (˜0.5 fold, p<0.05) (FIGS. 9a and b).

Since Mtd-L and Mtd-P expression has been shown to increase in preeclamptic placentae as well as under conditions of oxidative stress (Soleymanlou, 2005), their expression was herein examined in conditions of chronic hypoxia. In contrast to Mcl-1L, Mtd-L mRNA expression was not statistically different between the various groups. The number of Mtd-P transcripts was significantly decreased in HA samples relative to MA (4 fold, p<0.0001) and SL tissues (0.5 fold, p=0.04) (FIG. 9d).

The protein isoform profile of Mtd and Mcl-1 was next investigated in HA and control (MA and SL) placentae. Similar to Mcl-1 mRNA expression, Mcl-1L protein expression was increased in HA and to a lesser extent in MA when compared to SL samples (FIG. 9e). Both Mcl-1Lc and Mcl-1S molecules were hardly detectable at the protein level in SL, MA and HA samples with no apparent change in expression between SL, MA and HA placental tissues (FIG. 9e). Although expression of all known Mtd proteins (L: ˜28 kDa, S: ˜18 kDa and P: ˜15 kDa) could be observed, no apparent difference in Mtd expression was noted between the conditions tested (FIG. 9f).

Mcl-1 immunoreactivity in placental sections from SL, MA and HA samples was predominantly observed in trophoblast cell layers (FIG. 9g, top panels). Mcl-1 staining intensity was markedly increased in HA sections relative to MA and SL samples, corroborating the results of Mcl-1 immunoblotting. Stromal regions were Mcl-1 negative. Similar to Mcl-1, Mtd was predominantly expressed in trophoblast cell layers although its expression was generally low. A slight decrease in Mtd trophoblast immunoreactivity was noted in HA sections relative to SL or MA (FIG. 9g, bottom panels).

### Caspase Activation in In Vivo Chronic and Pathological Placental Hypoxia.

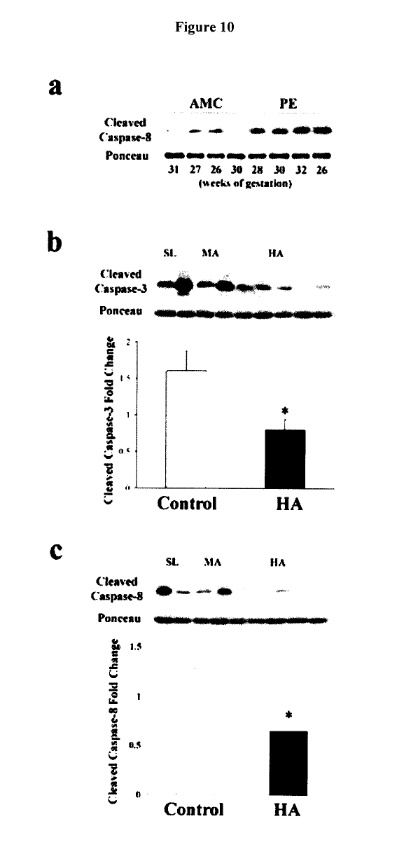

Excessive cell death via death receptor is a well-know phenomenon occurring frequently in trophoblast cells of preeclamptic placentae (Conrad, 1997; Hsu, 2001). While this death pathway was described previously, downstream events related to caspase 8 activation have not been explored in PE tissues. As such the expression of cleaved caspase-8 in early onset PE was analyzed and it was found that relative to age-matched control tissues it is increased (FIG. 10a), in line with increased expression/activation of caspase-3 (Aban, 2004).

Since a shift in the Mtd/Mcl-1 apoptotic rheostat towards increased pro-survival and decreased death-inducing isoforms was observed in HA, markers of trophoblast cell death in HA and control placentae were also measured. Expression of cleaved caspase-3, a known marker of trophoblast apoptosis (Soleymanlou, 2005; Hung, 2002), was reduced in HA by 50% (p=0.0297) relative to controls (MA and SL) (FIG. 10b). Expression of cleaved caspase-8 was also assessed in chronically hypoxic placental tissues. Similar to cleaved caspase-3, expression of activated caspase-8 in HA was decreased by 40% relative to controls (MA and SL) (p=0.0335) (FIG. 10c).

### Trophoblast Cell Fusion in Pathologic and In Vivo Chronic Placental Hypoxia.

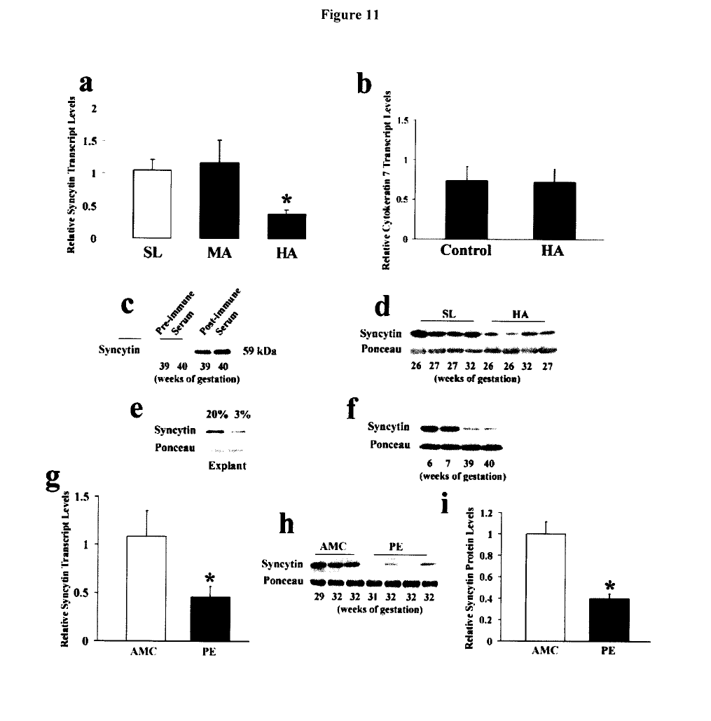

As placentae from high altitude pregnancies exhibit thinning of trophoblast membranes possibly due to reduced trophoblast cell death/turnover, the expression of the trophoblast differentiation marker called “syncytin” was next investigated (Frendo, 2003; Mi, 2000), a typical marker for cytotrophoblast fusion into syncytium. Using qRT-PCR the relative transcript expression of syncytin was measured in placental tissues from SL, MA and HA pregnancies. HA samples relative to MA or SL tissues, have significantly reduced syncytin expression (FIG. 11a). To ascertain that decreased syncytin expression in HA was not due to a shift in trophoblast cell population, expression of trophoblast-specific cytokeratin 7 was measured and found to be unchanged between HA and control tissues (FIG. 11b).

To further characterize the expression of the fusogenic syncytin protein, a polyclonal anti-syncytin antibody was generated. Antibody specificity was tested by exposing placental protein lysates from the same third trimester samples to pre-immune and post-immune serum in western blot analyses. A specific band at around 59 kDa (the theoretical molecular weight of syncytin) was recognized only when the post-immunized serum of rabbits was used (FIG. 11c). This band disappeared when competed with the peptide used for immunization (not shown). Similar to its messenger expression, syncytin protein expression was decreased in conditions of chronic placental hypoxia as determined by expression in HA placentae relative to low altitude control samples (FIG. 11d). Exposing first trimester explants to 3% O₂ also resulted in decreased syncytin expression (FIG. 11e). Finally, syncytin expression was tested in pathologic conditions of placental hypoxia and it was found that syncytin mRNA expression was reduced in preeclamptic placentae relative to controls (FIG. 11f), in agreement with previous reports (Knerr, 2002; Keith, 2002; Lee, 2001). In addition to decreased syncytin mRNA expression between PE and AMC, syncytin protein expression in preeclampsia was also reduced relative to age-matched control tissues (FIGS. 11g and h).

### Discussion

During normal placentation, a balance between proliferation, differentiation and apoptosis is required to regulate cellular homeostasis and maintenance of proper placental function. While it is now established that apoptosis is a key physiological event in placental tissue morphogenesis, the underlying mechanisms coordinating cell death in normal and abnormal placentation remains to be elucidated. Data presented herein demonstrate that pathological oxygenation vs. physiological low pO₂ induces a detrimental switch in the trophoblast Mcl-1/Mtd apoptotic rheostat, likely contributing to dysregulated cell death and leading to placental pathology. In particular, it was demonstrated that: 1) Mcl-1 isoform expression as well as its caspase-3-mediated cleavage are oxygen-dependent events in the human placenta, 2) Mcl-1 isoform expression is tilted towards expression of cell death promoting isoforms in severe early-onset preeclampsia and towards protective isoforms in altitude-induced chronic placental hypoxia and 3) trophoblast cell fusion is reduced in both physiological and pathological placental hypoxia, but trophoblast cell death is decreased in high altitude pregnancies and increased in preeclampsia.

Mcl-1 is a highly regulated molecule both at transcript and protein levels. The PEST domain in Mcl-1L and Mcl-1S contains aspartic acid residues, which are substrate for caspase-3 mediated cleavage (Han, 2005). This cleavage is a unique regulatory mechanism conferring differential Mcl-1 protein function. Mcl-1L is a potent anti-apoptotic molecule believed to sequester other members of the pro-apoptotic channel forming Bcl-2 subfamily. Recently, it was shown that Bak is specifically sequestered by Mcl-1L to prevent and neutralize its apoptotic function (Willis, 2005). Mcl-1L may prevent Mtd function in the same manner, since it is known that Mcl-1L inhibits the pro-apoptotic function of Mtd (Hsu, 1997). Mcl-1 protein stability is also trivial for its interaction capability and ultimately its anti-apoptotic function, as Mcl-1L protein is prone to caspase-mediated cleavage resulting in loss of its protective function and accumulation of Mcl-1Lc (p29), a cell death inducing fragment (Herrant, 2004; Michels, 2004; Weng, 2005; Snowden, 2003; Han, 2005; Han, 2004).

Oxygen is a potent regulator of apoptotic cell death. Several Bcl-2 family members, including BH3-only pro-apoptotic molecules Nix and Nip, have been shown to be directly regulated by oxygen via HIF-1 (Bruick, 2000; Sowter, 2001). This dimeric transcription factor is composed of an oxygen-labile moiety (HIF-1α subunit) and a constitutively expressed moiety (HIF-1α). HIF-1 regulates expression of many genes whose promoters contain hypoxia-responsive elements (HREs) under conditions of reduced oxygenation including Mcl-1 (Piret, 2004). The observation of increased Mcl-1L expression under in vitro or in vivo reduced oxygenation is consistent with the HIF-1 mediated Mcl-1L regulation (Piret, 2004). Hence, transcriptional regulation of Mcl-1 and differential expression of its isoforms may be attributed to specific oxygen conditions experienced by the placental tissue. Data reported herein are the first evidence of regulation of Mcl-1 expression and cleavage by oxygen in a human organ and importantly dysregulation of these events in a human disorder.

In severe early-onset preeclampsia, excess trophoblast cell death, likely induced by hypoxia (≅10 mmHg or <1-2% O₂) (Levy, 2000; Kilani, 2003) or intermittent oxygenation (Hung, 2002) increases trophoblast shedding, which is believed to generate a Thl-type maternal inflammatory response and generalized maternal endothelial cell injury (Saito, 2003; Sargent, 2003). A tilt in Mcl-1 expression towards generation of death inducing molecules in severe preeclamptic placentae combined with increased expression of killer Mtd-P isoform in this disease (Soleymanlou, 2005), initiates a detrimental pathologic switch towards trophoblast demise accompanied by increased shedding. In contrast to preeclamptic placentae, HA placental tissues, which experience chronic reduced oxygenation, exhibit decreased markers of apoptosis and increased Mcl-1L expression. In support of the molecular findings, previous studies have reported decreased formation of syncytial knots in high-altitude placentae relative to lower altitudes (Mayhew, 2002), again suggesting slowed apoptotic-mediated trophoblast turnover in chronic placental hypoxia. Hence conditions of chronic hypoxia may provide an adaptive response by minimizing the burden of apoptotic-mediated trophoblast shedding in these pregnancies. This may be achieved by increased placental expression of anti-apoptotic molecules in HA tissues relative to controls

Similar to increased Mtd-L and Mtd-P expression in early-onset severe preeclampsia (Soleymanlou, 2005), cleavage of Mcl-1L may be another unique placental feature of the early severe form of this disease, which does not occur in control aged-matched patients, in late onset preeclampsia or in other placental pathologies, including IUGR and essential hypertension. This observation once again reiterates the need and importance for proper pathological classification of preeclampsia, conceivably due to different underlying etiologies involved in the pathogenesis of this multifaceted and often misconstrued disorder.

Normal physiologic regulation of trophoblast cell fusion proceeds via an apoptotic-mediated mechanism where mononucleated cytotrophoblast cells fuse to maintain a multinuleacted syncytiotrophoblast cell layer (Huppertz, 1999). The rate of cyto- to syncytiotrophoblast differentiation decelerates with normal advancing gestation as cytotrophoblasts become sparse towards the end of gestation. Normal sea-level term placentae have a well-defined syncytiotrophoblast layer and few remaining cytotrophoblasts, which is in contrast to HA placentae showing abnormal thinning of the syncytiotrophoblast layer and a hyperproliferative cytotrophoblast phenotype. Thinning of the syncytiotrophoblast cell layer may be a physiological mechanism in high-altitude placentae to ensure maximal delivery of oxygen to the fetus (Zamudio, 2003). The mechanism for this thinning is presently not fully understood although it may be due to aberrant syncytial renewal under conditions of reduced oxygenation as previously proposed (Mayhew, 2002; Jackson, 1987; Alsat, 1996).

Decreased expression of active caspase-8, a promoter of trophoblast cell fusion (Black, 2004), in conjunction with reduced trophoblast cell death in high altitude placentae suggests a slowdown of syncytial formation and consequently trophoblast shedding during chronic placental hypoxia. Interestingly, in pathological placental hypoxia (preeclampsia), increased caspase-3 (Aban, 2004; Hung, 2002) and increased caspase-8 activation as reported herein indicate increased apoptosis. Activation of caspase-8 in PE could be due to aberrant expression of its upstream activators in this disease, such as activation of death receptors by FasL and TNFβ as previously reported (Conrad, 1997; Hsu, 2001; Leung, 2001; Allaire, 2000; Crocker, 2003; Ishihara, 2002; Soleymanlou, 2005). As such, active caspase-8 in PE likely mediates accelerated trophoblast turnover hence resulting in increased trophoblast shedding.

Decreased syncytin expression, a key regulator of trophoblast cell fusion (Mi, 2000; Frendo, 2003), in HA may also negatively impact normal trophoblast differentiation resulting in thinning of the placental syncytium. Studies have shown that in conditions of reduced oxygenation, syncytin as well as its binding receptor (ASCT2) are downregulated relative to standard oxygenation conditions (Kudo, 2003). Decreased syncytin expression in pregnancies complicated by preeclampsia has also been reported (Knerr, 2002; Lee, 2001). These findings support the observations of reduced syncytin expression in conditions of altitude-induced placental hypoxia or pathological hypoxia (PE) relative to control scenarios. As such impaired trophoblast fusion in HA placentae may limit de novo syncytial synthesis and maintenance at the expense of normal shedding/deportation of syncytial debris. Hence, this imbalance in the dynamic rate of syncytial renewal and shedding may provide a molecular explanation with respect to the thinned syncytial phenotype observed in high altitude placentae.

In conclusion, this study provides evidence that the Mcl-1/Mtd rheostat regulates trophoblast apoptosis in a different manner under physiological and pathological conditions of placental hypoxia. As well, it was demonstrated that while both preeclampsia and high altitude placentation are characterized by aberrant villous trophoblast differentiation, possibly due to downregulation of syncytin expression; only preeclamptic placentae experience an apoptotic-insult, believed to be responsible for accelerated trophoblast turnover. In high altitude pregnancies, decreased trophoblast cell death in conjunction with decreased trophoblast turnover/differentiation may present an important adaptive response to chronic placental hypoxia, as such improving the outcome of pregnancies under chronic reduced oxygenation. The impact of aberrant oxygenation on Mcl-1 isoform expression and stability in preeclamptic placental tissues may influence events leading to the clinical manifestations of this disease. el regulator of human trophoblast cell death in preeclampsia.

### EXAMPLE 8

### Ceruloplasmin

Ceruloplasmin (Cp) is a 132-kDa copper protein that is abundant in serum. Several lines of evidence suggest that Cp has an important role in iron metabolism. First, Cp catalyzes the conversion of Fe²⁺ to Fe³⁺ and is the major ferroxidase in plasma. This function is thought to be critical for loading of iron into apo-transferrin. Second, Cp is likely to be important in iron transport and homeostasis. The final evidence that Cp is involved in iron metabolism comes from the observation that alterations in serum iron status are often accompanied by changes in serum Cp (and Copper) (1). Cp is an enhancer of erythropoiesis.

Placental tissue samples collected from three different type of pathological pregnancies were studied:
1. Pregnancies complicated by severe Preeclampsia (ePE), a maternal syndrome characterized by hypertension (systolic blood pressure≧140 mmHg; diastolic blood pressure≧90 mmHg), proteinuria (≧300 mg/24 h) and preterm delivery according to ACOG guidelines.
2. Pregnancies complicated by severe Intrauterine Growth Restriction (sIUGR), disease characterized by fetal growth<5° centile according to gestational age and sex, pathological umbilical Doppler flow velocimetry waveform (Absent End Diastolic Flow), pathological bilateral uterine Doppler.
3. Preeclamptic pregnancies complicated by IUGR.

All these pathologies have as a common feature a placental hypoxic environment.

Placental Ceruloplasmin (pCp) is differentially expressed among the three groups: pCp levels are dramatically decreased in placentae from sIUGR and PE-IUGR pregnancies in comparison to their age-matched controls. Interestingly, an increase in pCp levels was found in PE pregnancies.

Moreover, Cp expression was studied in placentae from high altitude pregnancies (HA), a unique model of physiological adaptation to chronic hypoxia. pCp levels are increased in HA placentae compared to sea levels controls.

The data indicate that Cp could be a useful marker to discriminate preeclamptic pregnancies to those complicated by IUGR. Furthermore, according to its biochemical features and to the data on HA pregnancies, Cp could have a role as a protective factor against hypoxia.

### EXAMPLE 9

### Activated TGFβ Signaling Regulates sFlt-1 Expression in IUGR Pregnancies

Elevated sFlt-1 expression found in preeclamptic and IUGR pregnancies have been implicated in the development of the maternal endothelial dysfunction typical of these disorders of pregnancies. However, little is known about the mechanisms that control sFlt-1 expression. It was demonstrated that during normal placentation, oxygen is a critical regulator of sFlt-1 expression. In addition, preeclampsia is associated with placental hypoxia. Since, preeclampsia is a multifactorial disease it is plausible that additional factors beside oxygen, such as transforming growth factor β (TGFβ) molecules, may contribute to the high levels of sFlt-1. Since sFlt-1 expression is elevated in severe IUGR placentae, the objectives of the present study were: 1) to examine the expression/activation of Smad2 and 3 (R-Smad) and inhibitory Smad7 (1-Smad) in normal and IUGR pregnancies; and 2) to explore the possibility that TGFβ 1/3 signaling via Smads regulate sFlt-1 expression.

Early onset (25-33 wks) severe IUGR with abnormal umbilical and uterine artery Doppler and from age-matched normal control tissues were collected. First trimester villous explants and BeWo and JEG-3 choriocarcinoma cell lines were cultured in standard conditions in the presence or absence of TGFβ1 or TGFβ 3 (5 ng/mL) for 24 hours. Message levels of sFlt-1 were measured by quantitative real-time PCR using specific TaqMan primers and probe. Protein expression of sFlt-1 was measured by Western blot using a polyclonal antibody against sFlt-1. In addition, protein lysates from placental tissues, explants and cells lines were analyzed with antibodies that recognize human Smad2, Smad3, Smad7 and phospho-Smad2/3.

Phosphorylation of both Smad2 and Smad3 was markedly increased in severe IUGR placentae compared to control tissue, while a decrease in Smad7 content was observed in IUGR placentae. Exposure of both villous explants and BeWo cells to TGFβ 1 and TGFβ 3 resulted in increased protein expression of phospho-Smad2 and 3, which was associated with a significant increase in sFlt-1 mRNA and protein levels. In contrast, JEG-3 cells responded to TGFβ treatment only by increasing phospho-Smad2, but not phospho-Smad3 and sFlt-1 mRNA and protein.

Conclusion: These data suggest that in severe IUGR pregnancies an activation of the TGFβ signaling pathway may in part be responsible for the increased sFlt-1 expression.

### EXAMPLE 10

### Hypoxia and TGFβs Regulate Endoglin Expression in Human Placenta

Endoglin, a co-receptor for transforming growth factor (TGF)-β1 and β3, is expressed in the human placenta where it plays a role in regulating early events of trophoblast differentiation. Recent evidence has indicated that in preeclamptic placentae endoglin expression is elevated and this is associated with high circulatory levels of its soluble form. Since preeclampsia may be the result of impaired oxygenation, the effect of oxygen on endoglin expression was examined using physiological and pathological models of placental hypoxia. Since TGFβ3 expression in the placenta is regulated by oxygen, the effect of TGFβ1 and TGFβ3 on endoglin expression was also examined.

Human placental tissue from first trimester and from discordant dichorionic and monochorionic twins (n=10) and age matched normal twins (n=5) were used as physiological and pathological models of placental hypoxia. In all twins the discordancy was above 25% and absence of end diastolic velocity was documented in the growth-restricted twin. Endoglin mRNA expression was measured by quantitative PCR analysis. Protein expression was measured by Western Blot analysis using endoglin antibodies. Villous explants (5-8 wks) were used to test the effect of both oxygen and TGFβ1 and TGFβ3 on endoglin expression.

Immunoblot analysis indicated that endoglin expression was high at 5-7 weeks of gestation, when oxygen tension is low and decreased after 10 weeks of gestation when oxygen tension increases. Real-time PCR showed significantly increased endoglin transcripts in IUGR discordant twins compared to their normal co-twins and to control non-discordant twins. Consistent with these mRNA findings, protein levels of both membrane and soluble endoglin were higher in the IUGR twin placentae relative to both the control co-twin and the normal twins. Exposure of villous explants to low oxygen (3% O₂) resulted in elevated expression of endoglin compared to standard conditions (20% O₂). Moreover, addition of TGFβ1 and TGFβ3 to villous explants also increased the expression of both membrane and soluble endoglin compared to non-treated explants.

The results demonstrate that oxygen regulates the expression of endoglin possibly via a mechanism that involves TGFβs. Reduced placental perfusion leading to placental hypoxia may contribute to the increased expression of endoglin in IUGR discordant twins.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

TABLE 1
Clinical Parameters of Participants

| | | Preterm | High | |
|---|---|---|---|---|
| | Term control | control | altitude | Early |
| | (TC) | (PTC) | (HA) | Preeclampsia (PE) |
| | (n = 15) | (n = 12) | (n = 16) | (n = 16) |
| Maternal Age (yr) | 28.5 ± 3.7 | 30.5 ± 4.8 | 27.6 ± 7.1 | 28.9 ± 6.0 |
| Gestational age (wk) | 39.2 ± 1.2 | 29.5 ± 4 | 39.8 ± 1.7 | 29.2 ± 2.9 |
| Gravidity/Parity (mean) | 2/1 | 2/0 | 2/0 | 2/1 |
| Blood pressure (mm Hg) | | | | |
| Systolic | 113 ± 8 | 114 ± 4 | 113 ± 13 | 181 ± 10 |
| Diastolic | 73 ± 8 | 70 ± 7 | 74 ± 8 | 111 ± 6.0 |
| Proteinuria (plus protein) | Absent | Absent | Absent | 3.4 ± 1.5 |
| Edema (% of patients) | Absent | Absent | Absent | 82% |
| Birth weight (g) | 3332 ± 346 | 1300 ± 730 | 3054 ± 276* | 1160 ± 352 |
| Mode of delivery (No.) | | | | |
| Cesarean section | 3 | 5 | 3 | 14 |
| Vaginal delivery | 12 | 7 | 13 | 2 |

Data are presented as mean ± SD.
Preterm control group also appears as age matched control (AMC)
*p < 0.05 high altitude vs. term control.
Term control (TC) appear also as sea level (SL)

TABLE 2
BioMarkers of HA, EPE, LPE, & IUGR

| High Altitude | Early Onset Severe | Late Onset Preeclampsia | Intra-Uterine Growth |
|---|---|---|---|
| (HA) | Preeclampsia (EPE) | (LPE) | Restriction (IUGR) |
| HIF ↑ ↑ ↑ | HIF ↑ ↑ ↑ | HIF - | HIF ↑ |
| TGFB3 ↑ SFLT 1 ↑ | TGFB3 ↑ SFLT ↑ | TGFB3 - SFLT - | TGFB3 ↑ SMAD2 ↑ |
| VEGF ↑ SMAD2/7 ↑ | VEGF ↑ SMAD2/7 ↑ | VEGF - SMAD2/7- | SMAD3 ↑ |
| MTDP/L- Mcl1L↑ | MTDP/L ↑ Mcl1l ↓ | MTDP/L- Mcl1L - | VEGF ↓ SFLT 2 ↑ |
| Mcl1c ↓ | Mcl1c ↑ | Mcl1c - | MTDP/L - Mcl1L - |
| | ENG↑ | | Mcl1c - |
| | | | ENG↑ |
| VHL ↑ | VHL ↓ | VHL - | VHL - |
| Siah1/2 -/↓ | Siah1/2 ↓ | Siah1/2 - | Siah1/2 ↑ |
| PHDs ↑ | PHDs ↓ | PHDs - | PHD ↑ |

Although HIF is highly increased in the pathological (PE, IUGR) and physiological (HA) hypoxic placentae, the mechanism underlying its regulation/degradation and activity are noticeably different in the various models tested.
This is the first molecular proof of differences in HA, IUGR and PE markers uniquely linked to these different conditions.

### CITATIONS FOR PUBLICATIONS

1. ACOG practice bulletin. Diagnosis and management of preeclampsia and eclampsia. Number 33, January 2002. American College of Obstetricians and Gynecologists. Int J Gynaecol Obstet 77: 67-75, 2002.
2. Ahmad S and Ahmed A. Elevated placental soluble vascular endothelial growth factor receptor-1 inhibits angiogenesis in preeclampsia. Circ Res 95: 884-891, 2004.
3. Banks R E, Forbes M A, Searles J, Pappin D, Canas B, Rahman D, Kaufmann S, Walters C E, Jackson A, Eves P, Linton G, Keen J, Walker J J and Selby P J. Evidence for the existence of a novel pregnancy-associated soluble variant of the vascular endothelial growth factor receptor, Flt-1. Mol Hum Reprod 4: 377-386, 1998.
4. Benirschke K. Recent trends in chorangiomas, especially those of multiple and recurrent chorangiomas. Pediatr Dev Pathol 2: 264-269, 1999.
5. Caniggia I, Mostachfi H, Winter J, Gassmann M, Lye S J, Kuliszewski M and Post M. Hypoxia-inducible factor-1 mediates the biological effects of oxygen on human trophoblast differentiation through TGFbeta(3). J Clin Invest 105: 577-587, 2000.
6. Caniggia I and Winter J L. Adriana and Luisa Castellucci Award lecture 2001. Hypoxia inducible factor-1: oxygen regulation of trophoblast differentiation in normal and pre-eclamptic pregnancies—a review. Placenta 23 Suppl A: S47-S57, 2002.
7. Caniggia I, Wu Y and Zamudio S. Overexpression of HIF-1 alpha in placentae from high altitude. Placenta 23: A.49-A.50, 2002.
8. Clark D E, Smith S K, He Y, Day K A, Licence D R, Corps A N, Lammoglia R and Chamock-Jones D S. A vascular endothelial growth factor antagonist is produced by the human placenta and released into the maternal circulation. Biol Reprod 59: 1540-1548, 1998.
9. Forsythe J A, Jiang B H, Iyer N V, Agani F, Leung S W, Koos R D and Semenza G L. Activation of vascular endothelial growth factor gene transcription by hypoxia-inducible factor 1. Mol Cell Biol 16: 4604-4613, 1996.
10. Gerber H P, Condorelli F, Park J and Ferrara N. Differential transcriptional regulation of the two vascular endothelial growth factor receptor genes. Flt-1, but not Flk-1/KDR, is up-regulated by hypoxia. J Biol Chem 272: 23659-23667, 1997.
11. Hempstock J, Jauniaux E, Greenwold N and Burton G J. The contribution of placental oxidative stress to early pregnancy failure. Hum Pathol 34: 1265-1275, 2003.
12. Hornig C, Barleon B, Ahmad S, Vuorela P, Ahmed A and Weich H A. Release and complex formation of soluble VEGFR-1 from endothelial cells and biological fluids. Lab Invest 80: 443-454, 2000.
13. Hung T H, Charnock-Jones D S, Skepper J N and Burton G J. Secretion of tumor necrosis factor-alpha from human placental tissues induced by hypoxia-reoxygenation causes endothelial cell activation in vitro: a potential mediator of the inflammatory response in preeclampsia. Am J Pathol 164: 1049-1061, 2004.
14. Hung T H, Skepper J N and Burton G J. In vitro ischemia-reperfusion injury in term human placenta as a model for oxidative stress in pathological pregnancies. Am J Pathol 159: 1031-1043, 2001.
15. Hung T H, Skepper J N, Charnock-Jones D S and Burton G J. Hypoxia-reoxygenation: a potent inducer of apoptotic changes in the human placenta and possible etiological factor in preeclampsia. Circ Res 90: 1274-1281, 2002.
16. Jaakkola P, Mole D R, Tian Y M, Wilson M I, Gielbert J, Gaskell S J, Kriegsheim A, Hebestreit H F, Mukherji M, Schofield C J, Maxwell P H, Pugh C W and Ratcliffe P J. Targeting of HIF-alpha to the von Hippel-Lindau ubiquitylation complex by O2-regulated prolyl hydroxylation. Science 292: 468-472, 2001.
17. Jauniaux E, Watson A L, Hempstock J, Bao Y P, Skepper J N and Burton G J. Onset of maternal arterial blood flow and placental oxidative stress. A possible factor in human early pregnancy failure. Am J Pathol 157: 2111-2122, 2000.
18. Kaufmann P, Mayhew T M and Charnock-Jones D S. Aspects of human fetoplacental vasculogenesis and angiogenesis. II. Changes during normal pregnancy. Placenta 25: 114-126, 2004.
19. Kendall R L and Thomas K A. Inhibition of vascular endothelial cell growth factor activity by an endogenously encoded soluble receptor. Proc Natl Acad Sci USA 90: 10705-10709, 1993.
20. Kingdom J C and Kaufmann P. Oxygen and placental vascular development. Adv Exp Med Biol 474: 259-275, 1999.
21. Koga K, Osuga Y, Yoshino O, Hirota Y, Ruimeng X, Hirata T, Takeda S, Yano T, Tsutsumi O and Taketani Y. Elevated serum soluble vascular endothelial growth factor receptor 1 (sVEGFR-1) levels in women with preeclampsia. J Clin Endocrinol Metab 88: 2348-2351, 2003.
22. Krussel J S, Casan E M, Raga F, Hirchenhain J, Wen Y, Huang H Y, Bielfeld P and Polan M L. Expression of mRNA for vascular endothelial growth factor transmembraneous receptors Flt1 and KDR, and the soluble receptor sflt in cycling human endometrium. Mol Hum Reprod 5: 452-458, 1999.
23. Levine R J, Maynard S E, Qian C, Lim K H, England L J, Yu K F, Schisterman E F, Thadhani R, Sachs B P, Epstein F H, Sibai B M, Sukhatme V P and Karumanchi S A. Circulating angiogenic factors and the risk of preeclampsia. N Engl J Med 350: 672-683, 2004.
24. Li H, Gu B, Zhang Y, Lewis D F and Wang Y. Hypoxia-induced increase in soluble Flt-1 production correlates with enhanced oxidative stress in trophoblast cells from the human placenta. Placenta 26: 210-217, 2005.
25. Livak K J and Schmittgen T D. Analysis of relative gene expression data using real-time quantitative PCR and the 2(-Delta Delta C(T)) Method. Methods 25: 402-408, 2001.
26. Lunell N O, Nylund L E, Lewander R and Sarby B. Uteroplacental blood flow in pre-eclampsia measurements with indium-113m and a computer-linked gamma camera. Clin Exp Hypertens B 1: 105-117, 1982.
27. Maynard S E, Min J Y, Merchan J, Lim K H, Li J, Mondal S, Libermann T A, Morgan J P, Sellke F W, Stillman I E, Epstein F H, Sukhatme V P and Karumanchi S A. Excess placental soluble fms-like tyrosine kinase 1 (sFlt1) may contribute to endothelial dysfunction, hypertension, and proteinuria in preeclampsia. J Clin Invest 111: 649-658, 2003.
28. Metzen E and Ratcliffe P J. HIF hydroxylation and cellular oxygen sensing. Biol Chem 385: 223-230, 2004.
29. Nagamatsu T, Fujii T, Kusumi M, Zou L, Yamashita T, Osuga Y, Momoeda M, Kozuma S and Taketani Y. Cytotrophoblasts up-regulate soluble fms-like tyrosine kinase-1 expression under reduced oxygen: an implication for the placental vascular development and the pathophysiology of preeclampsia. Endocrinology 145: 4838-4845, 2004.
30. Palmer S K, Moore L G, Young D, Cregger B, Berman J C and Zamudio S. Altered blood pressure course during normal pregnancy and increased preeclampsia at high altitude (3100 meters) in Colorado. Am J Obstet Gynecol 180: 1161-1168, 1999.
31. Rajakumar A, Brandon H M, Daftary A, Ness R and Conrad K P. Evidence for the functional activity of hypoxia-inducible transcription factors overexpressed in preeclamptic placentae. Placenta 25: 763-769, 2004.
32. Sargent I L, Germain S J, Sacks G P, Kumar S and Redman C W. Trophoblast deportation and the maternal inflammatory response in pre-eclampsia. J Reprod Immunol 59: 153-160, 2003.
33. Sasaki H, Ray P S, Zhu L, Galang N and Maulik N. Oxidative stress due to hypoxia/reoxygenation induces angiogenic factor VEGF in adult rat myocardium: possible role of NFkappaB. Toxicology 155: 27-35, 2000.
34. Sawano A, Takahashi T, Yamaguchi S, Aonuma M and Shibuya M. Flt-1 but not KDR/FIk-1 tyrosine kinase is a receptor for placenta growth factor, which is related to vascular endothelial growth factor. Cell Growth Differ 7: 213-221, 1996.
35. Soleymanlou N, Jurisica I, Nevo O, Ietta F, Zhang X, Zamudio S, Post M and Caniggia I. Molecular evidence of placental hypoxia in preeclampsia. J Clin Endocrinol Metab 90: 4299-4308, 2005.
36. Soleymanlou N, Wu Y, Wang J X, Todros T, Ietta F, Jurisicova A, Post M and Caniggia I. A novel Mtd splice isoform is responsible for trophoblast cell death in pre-eclampsia. Cell Death Differ 12: 441-452, 2005.
37. Thaler I, Manor D, Itskovitz J, Rottem S, Levit N, Timor-Tritsch I and Brandes J M. Changes in uterine blood flow during human pregnancy. Am J Obstet Gynecol 162: 121-125, 1990.
38. Trollmann R, Amann K, Schoof E, Beinder E, Wenzel D, Rascher W and Dotsch J. Hypoxia activates the human placental vascular endothelial growth factor system in vitro and in vivo: up-regulation of vascular endothelial growth factor in clinically relevant hypoxic ischemia in birth asphyxia. Am J Obstet Gynecol 188: 517-523, 2003.
39. Tsao P N, Wei S C, Su Y N, Chou H C, Chen C Y and Hsieh W S. Excess soluble fms-like tyrosine kinase 1 and low platelet counts in premature neonates of preeclamptic mothers. Pediatrics 116: 468-472, 2005.
40. Tsatsaris V, Goffin F, Munaut C, Brichant J F, Pignon M R, Noel A, Schaaps J P, Cabrol D, Frankenne F and Foidart J M. Overexpression of the soluble vascular endothelial growth factor receptor in preeclamptic patients: pathophysiological consequences. J Clin Endocrinol Metab 88: 5555-5563, 2003.
41. Viero S, Chaddha V, Alkazaleh F, Simchen M J, Malik A, Kelly E, Windrim R and Kingdom J C. Prognostic value of placental ultrasound in pregnancies complicated by absent end-diastolic flow velocity in the umbilical arteries. Placenta 25: 735-741, 2004.
42. Zamudio S. The placenta at high altitude. High Alt Med Biol 4: 171-191, 2003.
43. Zhou Y, McMaster M, Woo K, Janatpour M, Perry J, Karpanen T, Alitalo K, Damsky C and Fisher S J. Vascular endothelial growth factor ligands and receptors that regulate human cytotrophoblast survival are dysregulated in severe preeclampsia and hemolysis, elevated liver enzymes, and low platelets syndrome. Am J Pathol 160: 1405-1423, 2002.
44. MacPhee, Lab Invest 2001, 81 (11) 1469-1483.
45. Caniggia, J Clin Invest 1999, 103 (12) 1641-1650.
46. Weng, J Biol Chem 2005, 280 (11) 10491-10500.
47. Snowden, Leukemia 2003, 7 (10) 1981-1989.
48. Herrant, Oncogene 2004, 23 (47) 7863-7873.
49. Han, J Biol Chem 2004, 279 (21) 22020-22029.
50. Han, J. Biol Chem 2005, 280 (16) 16383-16392.
51. Michels, Oncogene 2004, 23 (28) 4818-4827.
52. Conrad, Am J Reprod Immunol 1997, 37 (3) 240-249.
53. Aban, Tohoku J Exp Med 2004, 204 (3) 195-202.
54. Frendo, Mol Cell Biol 2003, 23 (10) 3566-3574.
55. Mi et al, Nature 2000, 403 (6771):785-9
56. Knerr, Am J Obstet Gynecol 2002, 186 (2) 210-213.
57. Keith, Am J Obstet Gynecol 2002, 187 (4) 1122-1123.
58. Lee, Placenta 2001, 22 (10) 808-812.
59. Willis, Genes Dev 2005, 19 (11) 1294-1305.
60. Hsu, Proc Natl Acad Sci USA 1997, 94 (23) 12401-12406.
61. Bruick, Proc Natl Acad Sci USA 2000, 97 (16) 9082-9087.
62. Sowter, Cancer Res 2001, 61 (18) 6669-6673.
63. Piret, Exp Cell Res 2004, 295 (2) 340-349.
64. Levy, Am J Physiol Cell Physiol 2000, 278 (5) 982-988.
65. Kilani, Placenta 2003, 24 (8-9) 826-834.
66. Saito, J Reprod Immunol 2003, 59 (2) 161-173.
67. Mayhew, Placenta 2002, 23 (2-3) 154-162.
68. Huppertz, Lab Invest 1999, 79 (12) 1687-1702.
69. Zamudio, High Alt Med Biol 2003, 4 (2) 171-191.
70. Jackson, Placenta 1987, 8 (5) 487-495.
71. Alsat, J Cell Physiol 1996, 168 (2) 346-353.
72. Black, Cell Death Differ 2004, 11 (1) 90-98.
73. Hsu, Plast Reconstr Surg 2001, 107 (7) 1789-1794.
74. Leung, Am J Obstet Gynecol 2001, 184 (6) 1249-1250.
75. Allaire, Obstet Gynecol 2000, 96 (2) 271-276.
76. Crocker, Am J. Pathol 2003, 162 (2) 637-643.
77. Ishihara, Am J Obstet Gynecol 2002, 186 (1) 158-166.
78. Kudo, Biochim Biophys Act 2003, 1638 (1) 63-71.

1. (canceled)

2. A method of detecting or diagnosing a condition requiring regulation of trophoblast cell death, differentiation, invasion, and/or cell fusion and turnover in a subject, the method comprising comparing:
levels of one or more polypeptide and/or polynucleotide markers associated with the condition that are extracted from a sample from the subject, wherein the markers comprise SMAD2, phospho-SMAD2, SMAD-3, phospho-SMAD3, SMAD7, and/or ceruloplasmin, and/or polynucleotides encoding SMAD2, phospho-SMAD2, SMAD-3, phospho-SMAD3, SMAD7, and/or ceruloplasmin; and
normal levels of expression of the markers in a control sample, wherein a significant difference in levels of markers relative to the corresponding normal levels, is indicative of the condition.

3. A method according to claim 2 wherein the markers are polypeptides.

4. A method according to claim 3 comprising:
contacting a biological sample obtained from a subject with one or more binding agents that specifically bind to the polypeptides or parts thereof; and
detecting in the sample amounts of polypeptides that bind to the binding agents, relative to a predetermined standard or cut-off value, and therefrom determining the presence or absence of the condition in the subject.

5. A method according to claim 4 wherein the binding agents are antibodies.

6. (canceled)

7. A method according to claim 2 wherein the markers are polynucleotides encoding SMAD2, SMAD-3, SMAD7, and/or ceruloplasmin.

8. A method according to claim 7 wherein the polynucleotides detected are mRNA.

9. A method according to claim 7 wherein the polynucleotides are detected by
contacting the sample with oligonucleotides that hybridize to the polynucleotides; and
detecting in the sample levels of nucleic acids that hybridize to the polynucleotides relative to a predetermined standard or cut-off value, and therefrom determining the presence or absence of a condition requiring regulation of trophoblast cell death, differentiation, invasion, and/or cell fusion and turnover n a subject in the subject.

10. A method according to claim 8 wherein the mRNA is detected using an amplification reaction.

11. A method according to claim 10 wherein the amplification reaction is a polymerase chain reaction employing oligonucleotide primers that hybridize to the polynucleotides, or complements of such polynucleotides.

12. A method according to claim 8 wherein the mRNA is detected using a hybridization technique employing oligonucleotide probes that hybridize to the polynucleotides or complements thereof, wherein the mRNA is detected by (a) isolating mRNA from the sample and combining the mRNA with reagents to convert it to cDNA; (b) treating the converted cDNA with amplification reaction reagents and primers that hybridize to the polynucleotides, to produce amplification products; (d) analyzing the amplification products to detect an amount of mRNA encoding one or more markers; and (e) comparing the amount of mRNA to an amount detected against a panel of expected values for normal tissue derived using similar primers.

13. (canceled)

14. A diagnostic composition comprising agents that bind to polypeptide markers associated with a condition requiring regulation of trophoblast cell death, differentiation, invasion, and/or cell fusion and turnover, or hybridize to polynucleotides encoding such polypeptide markers, wherein the markers comprise SMAD2, phospho-SMAD2, SMAD-3, phospho-SMAD3, SMAD7, and/or ceruloplasmin, and/or polynucleotides encoding SMAD2, phospho-SMAD2, SMAD-3, phospho-SMAD3, SMAD7, and/or ceruloplasmin.

15. (canceled)

16. A method according to claim 2 which further comprises detecting or comparing levels of sFlt, Mtd-L, Mtd-S, Mtd-P, Mcl-1 isoforms, TGFβ3, HIF1α, HIF-2α, endoglin, PHD1, PHD2, PHD3, VHL, Siah1/2, cullin 2, NEDD8, VEGF, FIH, syncytin, cleaved caspase, Fas, and/or p53, and/or polynucleotides encoding any of these polypeptides.

17. A method according to claim 2 wherein the markers are SMAD2, phospho-SMAD2, SMAD7, sFlt, Mtd-L, Mtd-P, Mcl-1c, Mcl-1L, TGFβ3, HIF1α, endoglin, PHD1, PHD2, PHD3, VHL, Siah1/2, and VEGF and/or polynucleotides encoding any of these polypeptides.

18. A method according to claim 2 wherein the markers are the biomarkers identified in Table 2.

19-23. (canceled)

24. A method according to claim 2 wherein the condition is preeclampsia.

25. (canceled)

26. A method according to claim 2 for diagnosing early onset peeclampsia in a subject comprising comparing levels of at least two, three, four, five, six, seven, eight, nine, ten, or all of SMAD2, phospho-SMAD2, SMAD-3, phospho-SMAD3, SMAD7, sFlt, ceruloplasmin, Mtd-P, Mtd-L, Mcl-1c, Mcl-11, TGFβ3, HIF1α, endoglin, PHD1, PHD2, Siah1/2, cleaved caspase, and VHL polypeptides, and/or polynucleotides encoding the polypeptides in a sample from the subject to the corresponding levels in a control.

27. A method according to claim 26 wherein the sample is taken from a subject in the first trimester of pregnancy, in particular before 14, 12, 10 or 8 weeks.

28. A method according to claim 26 wherein the control is a pre-term or normotensive age-matched control or a sample taken at a different stage of pregnancy.

29-36. (canceled)

37. A method according to claim 2 for diagnosing IUGR comprising comparing levels of HIF1α, TGFβ3, SMAD2, phospho-SMAD2, SMAD3, phospho-SMAD3, SMAD7, sFlt, MtdP/L, Siah1, Siah2, PHD1, PHD2, PHD3, VEGF, ceruloplasmin, endoglin, and/or VHL polypeptides, and/or polynucleotides encoding the polypeptides, in a sample from a subject to the corresponding levels in a control.

38-39. (canceled)

40. A kit for conducting a method of claim 2.

41. (canceled)
